(19) United States
(12) Patent Application Publication
(10) Pub. No.: US 2009/0099248 A1
(43) Pub. Date: Apr. 16, 2009

(54) INHIBITORS OF D-AMINO ACID OXIDASE

(75) Inventors: Michele L. R. Heffernan, Worcester, MA (US); Qun Kevin Fang, Wellesley, MA (US); Robert J. Foglesong, Durham, NC (US); Seth C. Hopkins, Clinton, MA (US); Cyprian O. Ogbu, Durham, NC (US); Mustapha Soukri, Raleigh, NC (US); Kerry L. Spear, Concord, MA (US)

Correspondence Address: MORGAN, LEWIS & BOCKIUS LLP (SF), One Market, Spear Street Tower, Suite 2800, San Francisco, CA, 94105, US

(73) Assignee: SEPRACOR INC., Marlborough, MA (US)

(21) Appl. No.: 12/016,954

(22) Filed: Jan. 18, 2008

Related U.S. Application Data
(60) Provisional application No. 60/885,588, filed on Jan. 18, 2007.

(51) Int. Cl. A61K 31/404; C07D 209/02; C07D 209/42; A61P 25/00; C12N 9/99; A61K 31/403

(52) U.S. Cl. 514/419; 548/516; 514/412; 548/492; 435/184

(57) ABSTRACT

The present invention provides novel inhibitors of the enzyme D-amino acid oxidase. The compounds of the invention are useful for treating or preventing diseases and/or condition, wherein modulation of D-serine levels, and/or its oxidative products, is effective in ameliorating symptoms. The invention further provides methods of enhancing learning, memory and/or cognition. For example, the invention provides methods for treating or preventing loss of memory and/or cognition associated with neurodegenerative diseases, such as Alzheimer's disease. The invention further provides methods for preventing loss of neuronal function characteristic of neurodegenerative diseases. In addition, methods are provided for the treatment or prevention of neuropsychiatric diseases (e.g., schizophrenia) and for the treatment or prevention of pain and ataxia.

### CROSS-REFERENCES TO RELATED APPLICATIONS

This application claims priority under 35 U.S.C. §119(e) to U.S. Provisional Patent Application No. 60/885,588 filed on Jan. 18, 2007, which is incorporated herein by reference in its entirety for all purposes.

### FIELD OF THE INVENTION

This invention relates to enzyme inhibitors, particularly inhibitors of D-amino acid oxidase (DAAO).

### BACKGROUND OF THE INVENTION

The enzyme D-amino acid oxidase (DAAO) metabolizes D-amino acids, and in particular, metabolizes D-serine in vitro at physiological pH. DAAO is expressed in the mammalian central and peripheral nervous system. D-Serine's role as a neurotransmitter is important in the activation of the N-methyl-D-aspartate (NMDA) selective subtype of the glutamate receptor, an ion channel expressed in neurons, here denoted as NMDA receptor.

NMDA receptors mediate many physiological functions. NMDA receptors are complex ion channels containing multiple protein subunits that act either as binding sites for transmitter amino acids and/or as allosteric regulatory binding sites to regulate ion channel activity. D-serine, released by glial cells, has a distribution similar to NMDA receptors in the brain and acts as an endogenous ligand of the allosteric “glycine” site of these receptors (Mothet et al., PNAS, 97:4926 (2000)), the occupation of which is required for NMDA receptor operation. D-serine is synthesized in brain through serine racemase and degraded by D-amino oxidase (DAAO) after release.

Small organic molecules, which inhibit the enzymatic cycle of DAAO, can be used to control the levels of D-serine, and thus can influence the activity of the NMDA receptor in the brain. NMDA receptor activity is important in a variety of disease states, such as schizophrenia, psychosis, ataxias, ischemia, several forms of pain including neuropathic pain, and deficits in memory and cognition.

DAAO inhibitors can also control production of toxic metabolites of D-serine oxidation, such as hydrogen peroxide and ammonia. Thus, these molecules can influence the progression of cell loss in neurodegenerative disorders. Neurodegenerative diseases are diseases in which CNS neurons and/or peripheral neurons undergo a progressive loss of function, usually accompanied by (and perhaps caused by) a physical deterioration of the structure of either the neuron itself or its interface with other neurons. Such conditions include Parkinson's disease, Alzheimer's disease, Huntington's disease and neuropathic pain. N-methyl-D-aspartate (NMDA)-glutamate receptors are expressed at excitatory synapses throughout the central nervous system (CNS). These receptors mediate a wide range of brain processes, including synaptic plasticity, that are associated with certain types of memory formation and learning. NMDA-glutamate receptors require binding of two agonists to induce neurotransmission. One of these agonists is the excitatory amino acid L-glutamate, while the second agonist, at the so-called “strychnine-insensitive glycine site”, is now thought to be D-serine. In animals, D-serine is synthesized from L-serine by serine racemase and degraded to its corresponding ketoacid by DAAO. Together, serine racemase and DAAO are thought to play a crucial role in modulating NMDA neurotransmission by regulating CNS concentrations of D-serine.

Known inhibitors of DAAO include benzoic acid, pyrrole-2-carboxylic acids, and indole-2-carboxylic acids, as described by Frisell, et al., J. Biol. Chem., 223:75-83 (1956) and Parikh et al., JACS, 80:953 (1958). Indole derivatives and particularly certain indole-2-carboxylates have been described in the literature for treatment of neurodegenerative disease and neurotoxic injury. EP 396124 discloses indole-2-carboxylates and derivatives for treatment or management of neurotoxic injury resulting from a CNS disorder or traumatic event or in treatment or management of a neurodegenerative disease. Several examples of traumatic events that can result in neurotoxic injury are given, including hypoxia, anoxia, and ischemia, associated with perinatal asphyxia, cardiac arrest or stroke. Neurodegeneration is associated with CNS disorders such as convulsions and epilepsy. U.S. Pat. Nos. 5,373,018; 5,374,649; 5,686,461; 5,962,496 and 6,100,289, to Cugola, disclose treatment of neurotoxic injury and neurodegenerative disease using indole derivatives. None of the above references mention improvement or enhancement of learning, memory or cognition.

WO 03/039540 to Heefner et al. and U.S. Patent Application Nos. 2005/0143443 to Fang et al. and 2005/0143434 to Fang et al. disclose DAAO inhibitors, including indole-2-carboxylic acids, and methods of enhancing learning, memory and cognition as well as methods for treating neurodegenerative disorders. Patent Application No. WO/2005/089753 discloses benzisoxazole analogs and methods of treating mental disorders, such as Schizophrenia. However, a need for additional drug molecules that are effective in treating memory defects, impaired learning, loss of cognition, and other symptoms related to NMDA receptor activity, remains. The present invention addresses this and other needs.

### SUMMARY OF THE INVENTION

The invention provides novel inhibitors of D-amino acid oxidase that are useful in the prevention and treatment of a variety of diseases and/or conditions including neurological disorders, pain, ataxia and convulsion.

In a first aspect, the present invention provides a compound having a structure according to Formula (VI):

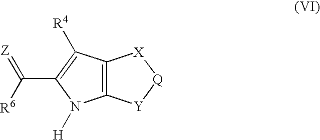

In Formula (VI), Z is a member selected from O and S. X, Q and Y are members independently selected from —CR¹R²—, C═O, C═S, C═NR³ and C═CR⁴⁰R⁴¹, with the proviso that at least one of X, Q and Y is other than —CH₂—. X and Q are optionally joined to form a 3-, 4- or 5-membered ring. Y and Q are optionally joined to form a 3-, 4- or 5-membered ring. X and Y, together with the atoms to which they are attached, are optionally joined to form a 5- to 7-membered ring thereby forming a bicyclic substructure.

In Formula (VI), R³ is a member selected from H, OR¹², acyl, NR¹²R¹³, SO₂R¹³, SOR¹³, substituted or unsubstituted alkyl, substituted or unsubstituted heteroalkyl, substituted or unsubstituted aryl, substituted or unsubstituted heteroaryl and substituted or unsubstituted heterocycloalkyl, wherein R¹² and R¹³ are members independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

In Formula (VI), R⁴ is a member selected from H, CF₃, F, Cl, Br, CN, OR¹⁴, NR¹⁴R¹⁵, C₄-C₆ unsubstituted alkyl, substituted or unsubstituted cycloalkyl, substituted or unsubstituted heterocycloalkyl, substituted or unsubstituted arylalkyl, substituted or unsubstituted heteroarylalkyl, cycloalkyl-substituted alkyl and heterocycloalkyl-substituted alkyl. R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

In Formula (VI), each R¹, each R², each R⁴⁰ and each R⁴¹ is a member independently selected from H, halogen, CN, CF₃, acyl, C(O)OR^{14′}, C(O)NR^{14′}R^{15′}, OR¹⁴, S(O)₂OR¹⁴, S(O)ₚR^{14′}, SO₂NR^{14′}R^{15′}, NR^{14′}R^{15′}, NR^{14′}C(O)R^{15′}, NR^{14′}S(O)₂R^{15′}, substituted or unsubstituted alkyl, substituted or unsubstituted heteroalkyl, substituted or unsubstituted aryl, substituted or unsubstituted heteroaryl and substituted or unsubstituted heterocycloalkyl, wherein p is an integer selected from 0 to 2. Adjacent R¹ and R², together with the atoms to which they are attached, are optionally joined to form a 3-, 4- or 5-membered ring. In one example, R¹ and R² are not joined to form a ring. R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, wherein R¹⁴ and R¹⁵, together with the atoms to which they are attached, are optionally joined to form a 5- to 7-membered ring. In one example, when X is C═O, then R⁴ is other than H. In another example, when X is C═O, then Q and Y are other than —CH₂—. In yet another example, when X is CHR¹, wherein R¹ is ethyl, propyl or butyl, then R⁴ is other than H. In a further example, when X is CHR¹, wherein R¹ is ethyl, propyl or butyl, then Q and Y are other than —CH₂—. In another example, when X is CHR¹, wherein R¹ is ethyl, propyl or butyl, then Y is other than C═O and CR¹R², wherein both R¹ and R² are —O-acyl (e.g., OAc). In a further example, when R⁴ is H, then X and Y are not both CR¹R², wherein both R¹ and R² are other than H (e.g., X and Y are not both C(Me)₂).

In Formula (VI), R⁶ is a member selected from OH and O⁻X⁺, wherein X⁺ is a cation.

Compounds of Formula (VI) include any enantiomer, diastereoisomer, racemic mixture, enantiomerically enriched mixture, and enantiomerically pure forms for each compound.

In one embodiment according to the above aspect, at least one of R¹, R², R³, R⁴⁰ and R⁴¹ in Formula (VI) has the formula:

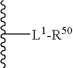

wherein R⁵⁰ is a member selected from substituted or unsubstituted aryl, substituted or unsubstituted heteroaryl, substituted or unsubstituted cycloalkyl, substituted or unsubstituted heterocycloalkyl and a fused ring system; and wherein L¹ is a linker moiety, which is a member selected from substituted or unsubstituted alkyl, substituted or unsubstituted heteroalkyl, substituted or unsubstituted aryl, substituted or unsubstituted heteroaryl and substituted or unsubstituted heterocycloalkyl.

In one example according to any of the above embodiments, at least one of R¹, R² and R³ has a formula, which is a member selected from:

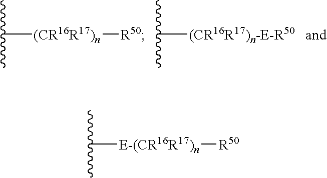

wherein n is an integer from 1 to 5. In the above structures, each E is a member independently selected from —O—, —S—, —NR⁴³—, —C(O)NR⁴³—, —NR⁴³C(O)—, —S(O)₂NR⁴³— and —NR⁴³S(O)₂—, wherein each R⁴³ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

R¹⁶ and R¹⁷ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, wherein two of R¹, R¹⁶ and R¹⁷ or two of R², R¹⁶ and R¹⁷, together with the carbon atoms to which they are attached, are optionally joined to form a 3- to 7-membered ring, wherein said ring is a member selected from substituted or unsubstituted cycloalkyl and substituted or unsubstituted heterocycloalkyl, and wherein said ring is optionally fused to R⁵⁰.

In another example according to any of the above embodiments, (CR¹⁶R¹⁷) is a member selected from —CH₂—, —CH₂CH₂— and —CH₂CH₂CH₂—.

In another example according to any of the above embodiments, R⁵⁰ is a member selected from substituted or unsubstituted aryl and substituted or unsubstituted heteroaryl.

In another example according to any of the above embodiments, R⁵⁰ is substituted or unsubstituted aryl and has the formula:

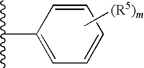

wherein m is an integer from 0 to 5. Each R⁵ is a member independently selected from H, halogen, CN, CF₃ hydroxy, alkoxy, acyl, C(O)OR¹⁸, OC(O)R¹⁸, NR¹⁸R¹⁹, C(O)NR¹⁸R¹⁹, NR¹⁸C(O)R²⁰, NR¹⁸SO₂R²⁰, S(O)₂R²⁰, S(O)R²⁰, substituted or unsubstituted alkyl, substituted or unsubstituted heteroalkyl, substituted or unsubstituted aryl, substituted or unsubstituted heteroaryl and substituted or unsubstituted heterocycloalkyl. Adjacent R⁵, together with the atoms to which they are attached, are optionally joined to form a ring (e.g., substituted or unsubstituted cycloalkyl, substituted or unsubstituted heterocycloalkyl, substituted or unsubstituted aryl and substituted or unsubstituted heteroaryl). R¹⁸ and R¹⁹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R²⁰ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. Two of R¹⁸, R¹⁹ and R²⁰, together with the atoms to which they are attached, are optionally joined to form a 5- to 7-membered ring.

In another example according to any of the above embodiments, the compound of the invention has a formula, which is a member selected from:

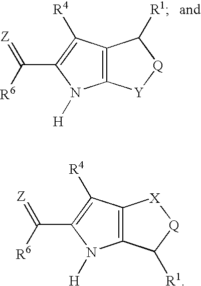

In another example according to any of the above embodiments, the compound of the invention has a formula, which is a member selected from:

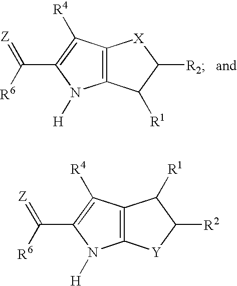

In another example according to any of the above embodiments, the compound of the invention has a structure, which is a member selected from:

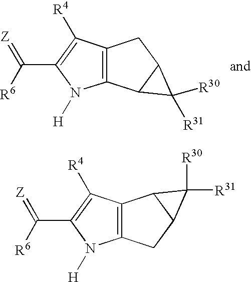

wherein R³⁰ and R³¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. For example, R³⁰ and R³¹ are not both methyl.

In another example according to any of the above embodiments, at least one of R³⁰ and R³¹ has the formula:

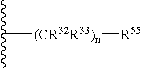

wherein each n is an integer from 0 to 5. R⁵⁵ is a member selected from substituted or unsubstituted aryl, substituted or unsubstituted heteroaryl, substituted or unsubstituted cycloalkyl and substituted or unsubstituted heterocycloalkyl. Each R³² and each R³³ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, wherein R³² and R³³, together with the carbon atom to which they are attached, are optionally joined to form a 3- to 7-membered ring, which is optionally fused to R⁵⁵

In another example according to any of the above embodiments, the compound of the invention has the formula:

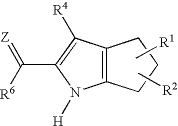

wherein at least one of R¹ and R² is other than H. In the above formula, adjacent R¹ and R², together with the atoms to which they are attached, are optionally joined to form a 3-, 4- or 5-membered ring.

In another example according to any of the above embodiments, the compound of the invention has formula, which is a member selected from:

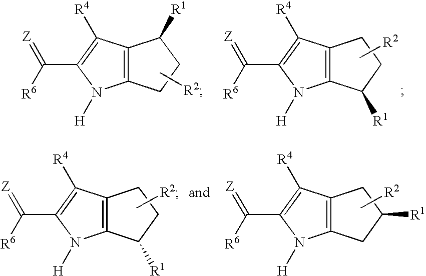

wherein R¹ is other than H and absolute stereochemistry with respect to R¹ is shown.

In another example according to any of the above embodiments, R¹ is substituted or unsubstituted alkyl.

In another example according to any of the above embodiments, R¹ is a member selected from substituted or unsubstituted methyl, substituted or unsubstituted ethyl, substituted or unsubstituted n-propyl, substituted or unsubstituted iso-propyl, substituted or unsubstituted n-butyl and substituted or unsubstituted iso-butyl.

In another example according to any of the above embodiments, R¹ is aryl-substituted alkyl or heteroaryl-substituted alkyl.

In another example according to any of the above embodiments, R¹ is alkyl substituted with a member selected from substituted or unsubstituted cycloalkyl and substituted or unsubstituted heterocycloalkyl.

In another example according to any of the above embodiments, Z is O.

In another example according to any of the above embodiments, R¹ and R² are members independently selected from H, F, methyl, ethyl, n-propyl, iso-propyl, n-butyl, iso-butyl, substituted or unsubstituted arylalkyl, substituted or unsubstituted heteroarylalkyl, cycloalkyl-substituted alkyl and heterocycloalkyl-substituted alkyl, wherein a cycloalkyl or heterocycloalkyl group is optionally substituted.

In another example, the invention provides a pharmaceutical composition including a compound of the invention (e.g., any of the compounds described in any of the above embodiments), or a pharmaceutically acceptable salt thereof, and a pharmaceutically acceptable carrier.

In another example, the invention provides a composition including a first stereoisomer and at least one additional stereoisomer of a compound of the invention (e.g., any of the compounds described in any of the above embodiments) wherein the first stereoisomer is present in an enantiomeric or diastereomeric excess of at least 80% relative to the at least one additional stereoisomer.

In a second aspect, the invention provides a method for treating or preventing a condition which is a member selected from a neurological disorder, pain, ataxia and convulsion. The method includes administering to a subject in need thereof a therapeutically effective amount of a compound according to Formula (I):

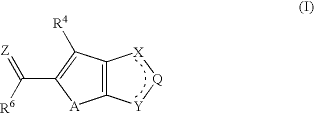

wherein Z is a member selected from O and S. A is a member selected from NR⁷, S and O. X, Q and Y are members independently selected from O, S, NR³, CR¹, —(CR¹R²)_{q}—, C═O, C═S, C═NR³ and C═CR⁴⁰R⁴¹, wherein q is an integer selected from 1 and 2. In Formula (I), the ring, which includes Q, X and Y is a non-aromatic ring. X and Q are optionally joined to form a 3- to 7-membered ring. Y and Q are optionally joined to form a 3- to 7-membered ring. X and Y, together with the atoms to which they are attached, are optionally joined to form a 5- to 7-membered ring thereby forming a bicyclic substructure.

In Formula (I), R³ and R⁷ are members independently selected from H, OR¹², acyl, NR¹²R¹³, SO₂R¹³, SOR¹³, substituted or unsubstituted alkyl, substituted or unsubstituted heteroalkyl, substituted or unsubstituted aryl, substituted or unsubstituted heteroaryl and substituted or unsubstituted heterocycloalkyl, wherein R¹² and R¹³ are members independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

In Formula (I), R⁴, each R¹, each R², each R⁴⁰ and each R⁴¹ are members independently selected from H, halogen, CN, CF₃, acyl, C(O)OR¹⁴, C(O)NR¹⁴R¹⁵, OR¹⁴, S(O)₂OR¹⁴, S(O)ₚR¹⁴, SO₂NR¹⁴R¹⁵, NR¹⁴R¹⁵, NR¹⁴C(O)R¹⁵, NR¹⁴S(O)₂R¹⁵, substituted or unsubstituted alkyl, substituted or unsubstituted heteroalkyl, substituted or unsubstituted aryl, substituted or unsubstituted heteroaryl and substituted or unsubstituted heterocycloalkyl, wherein p is an integer selected from 0 to 2. R¹ and R², together with the atoms to which they are attached, are optionally joined to form a 3- to 7-membered ring. R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R¹⁴ and R¹⁵, together with the atoms to which they are attached, are optionally joined to form a 5- to 7-membered ring.

In Formula (I), R⁶ is a member selected from OR⁸, O⁻X⁺, NR⁹R¹⁰, NR⁹NR^{9′}R¹⁰, NR⁹OR¹⁰, NR⁹SO₂R¹¹, wherein X⁺ is a cation. R⁶ and R⁷, together with the atoms to which they are attached, are optionally joined to form a 5- to 7-membered ring. R⁸ is a member selected from the group consisting of H, substituted or unsubstituted alkyl, substituted or unsubstituted heteroalkyl, substituted or unsubstituted aryl, substituted or unsubstituted heteroaryl, substituted or unsubstituted heterocycloalkyl and a single negative charge. R⁹, R^{9′} and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R¹¹ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. At least two of R⁸, R⁹, R^{9′}, R¹⁰ and R¹¹, together with the atoms to which they are attached, are optionally joined to form a 5- to 7-membered ring.

In one example according to any of the above embodiments, in Formula (I), A is NR⁷ (e.g., NH). In another example according to any of the above embodiments, in Formula (I), R⁶ is OR⁸ or O⁻X⁺. For example, R⁸ is a member selected from H and a single negative charge. In another example according to any of the above embodiments, in Formula (I), R¹ and R² are members independently selected from H, F, methyl, ethyl, n-propyl, iso-propyl, n-butyl, iso-butyl, substituted or unsubstituted arylalkyl, substituted or unsubstituted heteroarylalkyl, substituted or unsubstituted cycloalkyl-substituted alkyl and substituted or unsubstituted heterocycloalkyl-substituted alkyl. In another example according to any of the above embodiments, in Formula (I), at least one of X, Q and Y is other than —CH₂—.

Compounds of Formula (I) include any enantiomer, diastereoisomer, racemic mixture, enantiomerically enriched mixture, and enantiomerically pure form of each compound.

In one example, the compound of Formula (I) has a structure according to Formula (VI):

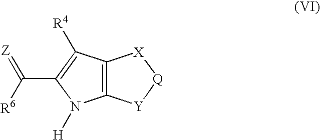

wherein Z, R⁴, R⁶, X, Q and Y are defined as outlined herein above. All exemplary embodiments outlined herein above for Formula (VI), equally apply to the compounds of this paragraph and the methods of the invention.

The invention further provides a method of enhancing cognition in a mammalian subject (e.g., a human patient). The method includes administering to the subject an effective amount of a compound of the invention. The compound can be any compound described herein above. In one example, the compound is a compound according to Formula (I). In another example, the compound is a compound according to Formula (VI). Any embodiments described herein above for Formula (I) and Formula (VI) equally apply to the method of this paragraph.

The invention further provides a method of inhibiting D-amino acid oxidase (DAAO) activity, said method comprising contacting said DAAO with a compound of the invention, wherein the compound can be any compound described herein above. In one example, the compound is a compound according to Formula (I). In another example, the compound is a compound according to Formula (VI). Any embodiments described herein above for Formula (I) and Formula (VI) equally apply to the method of this paragraph.

The invention further provides a method of increasing D-serine level in the brain (e.g., cerebellum) of a mammal (e.g., a rodent or a human). The method includes administering to the mammal an effective amount of a compound of the invention, wherein the compound can be any compound described herein above. In one example, the compound is a compound according to Formula (I). In another example, the compound is a compound according to Formula (VI). Any embodiments described herein above for Formula (I) and Formula (VI) equally apply to the method of this paragraph.

### DETAILED DESCRIPTION OF THE INVENTION

### I. Definitions

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which can be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl” with the difference that the heteroalkyl group, in order to qualify as an alkyl group, is linked to the remainder of the molecule through a carbon atom. Alkyl groups that are limited to hydrocarbon groups are termed “homoalkyl”.

The term “alkenyl” by itself or as part of another substituent is used in its conventional sense, and refers to a radical derived from an alkene, as exemplified, but not limited, by substituted or unsubstituted vinyl and substituted or unsubstituted propenyl. Typically, an alkenyl group will have from 1 to 24 carbon atoms, with those groups having from 1 to 10 carbon atoms being preferred.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, S, B and P and wherein the nitrogen and sulfur atoms can optionally be oxidized and the nitrogen heteroatom can optionally be quaternized. The heteroatom(s) can be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. A “cycloalkyl” or “heterocycloalkyl” substituent can be attached to the remainder of the molecule directly or through a linker. An exemplary linker is alkylene. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (e.g., from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring can optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring can optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed can optionally be replaced with a double bond. Alternatively, two of the substituents on adjacent atoms of the aryl or heteroaryl ring can optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are independently selected from hydrogen or substituted or unsubstituted (C₁-C₆)alkyl.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems can include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si) and boron (B).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

As used herein, the term “aromatic ring” or “non-aromatic ring” is consistent with the definition commonly used in the art. For example, aromatic rings include phenyl and pyridyl. Non-aromatic rings include cyclohexanes.

The phrase “therapeutically effective amount” as used herein means that amount of a compound, material, or composition comprising a compound of the present invention which is effective for producing a desired therapeutic effect, at a reasonable benefit/risk ratio applicable to any medical treatment.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts.

When a residue is defined as “O⁻”, then the formula is meant to optionally include an organic or inorganic cationic counterion. For example, the resulting salt form of the compound is pharmaceutically acceptable.

The neutral forms of the compounds are, for example, regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. For instance, prodrugs for carboxylic acid analogs of the invention include a variety of esters. In an exemplary embodiment, the pharmaceutical compositions of the invention include a carboxylic acid ester. In another exemplary embodiment, the prodrug is suitable for treatment/prevention of those diseases and conditions that require the drug molecule to cross the blood brain barrier. In a preferred embodiment, the prodrug enters the brain, where it is converted into the active form of the drug molecule. In another example, a prodrug is used to enable an active drug molecule to reach the inside of the eye after topical application of the prodrug to the eye. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention can exist in multiple crystalline or amorphous forms (“polymorphs”). In general, all physical forms are of use in the methods contemplated by the present invention and are intended to be within the scope of the present invention. “Compound or a pharmaceutically acceptable salt, hydrate, polymorph or solvate of a compound” intends the inclusive meaning of “or”, in that materials meeting more than one of the stated criteria are included, e.g., a material that is both a salt and a solvate is encompassed.

The compounds of the present invention can contain unnatural proportions of atomic isotopes at one or more of the atoms that constitute such compounds. For example, the compounds can be radiolabeled with radioactive isotopes, such as for example tritium (3H), iodine-125 (¹²⁵I) or carbon-14 (¹⁴C). All isotopic variations of the compounds of the present invention, whether radioactive or not, are intended to be encompassed within the scope of the present invention.

In the context of the present invention, compounds that are considered to possess activity as DAAO inhibitors are those displaying 50% inhibition of the enzymatic activity of DAAO (IC₅₀) at a concentration of not higher than about 100 μM. For example, the IC₅₀ is not higher than about 10 μM, not higher than about 1 μM or not higher than about 100 nM. In one example, the IC₅₀ is not higher than about 25 nM.

The term “neurological disorder” refers to any undesirable condition of the central or peripheral nervous system of a mammal. The term “neurological disorder” includes neurodegenerative diseases (e.g., Alzheimer's disease, Parkinson's disease and amyotrophic lateral sclerosis), neuropsychiatric diseases (e.g. schizophrenia and anxieties, such as general anxiety disorder). Exemplary neurological disorders include MLS (cerebellar ataxia), Huntington's disease, Down syndrome, multi-infarct dementia, status epilecticus, contusive injuries (e.g. spinal cord injury and head injury), viral infection induced neurodegeneration, (e.g. AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any undesirable condition associated with the disorder. For instance, a method of treating a neurodegenerative disorder includes methods of treating loss of memory and/or loss of cognition associated with a neurodegenerative disorder. Such method would also include treating or preventing loss of neuronal function characteristic of neurodegenerative disorder.

“Pain” is an unpleasant sensory and emotional experience. Pain classifications have been based on duration, etiology or pathophysiology, mechanism, intensity, and symptoms. The term “pain” as used herein refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (see, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia.

“Somatic” pain, as described above, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

“Neuropathic pain” is a heterogeneous group of neurological conditions that result from damage to the nervous system. “Neuropathic” pain, as described above, refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Peripheral neuropathic pain includes without limitation diabetic neuropathy (also called diabetic peripheral neuropathic pain, or DN, DPN, or DPNP), post-herpetic neuralgia (PHN), and trigeminal neuralgia (TGN). Central neuropathic pain, involving damage to the brain or spinal cord, can occur following stroke, spinal cord injury, and as a result of multiple sclerosis. Other types of pain that are meant to be included in the definition of neuropathic pain include pain from neuropathic cancer pain, HIV/AIDS induced pain, phantom limb pain, and complex regional pain syndrome. In a preferred embodiment, the compounds of the invention are of use for treating neuropathic pain.

Common clinical features of neuropathic pain include sensory loss, allodynia (non-noxious stimuli produce pain), hyperalgesia and hyperpathia (delayed perception, summation, and painful aftersensation). Pain can be a combination of nociceptive and neuropathic types, for example, mechanical spinal pain and radiculopathy or myelopathy.

“Acute pain”, is the normal, predicted physiological response to a noxious chemical, thermal or mechanical stimulus typically associated with invasive procedures, trauma and disease. It is generally time-limited, and can be viewed as an appropriate response to a stimulus that threatens and/or produces tissue injury. “Acute pain”, as described above, refers to pain which is marked by short duration or sudden onset.

“Chronic pain” occurs in a wide range of disorders, for example, trauma, malignancies and chronic inflammatory diseases such as rheumatoid arthritis. Chronic pain usually lasts more than about six months. In addition, the intensity of chronic pain can be disproportionate to the intensity of the noxious stimulus or underlying process. “Chronic pain”, as described above, refers to pain associated with a chronic disorder, or pain that persists beyond resolution of an underlying disorder or healing of an injury, and that is often more intense than the underlying process would predict. It can be subject to frequent recurrence.

“Inflammatory pain” is pain in response to tissue injury and the resulting inflammatory process. Inflammatory pain is adaptive in that it elicits physiologic responses that promote healing. However, inflammation can also affect neuronal function. Inflammatory mediators, including PGE₂ induced by the COX2 enzyme, bradykinins, and other substances, bind to receptors on pain-transmitting neurons and alter their function, increasing their excitability and thus increasing pain sensation. Much chronic pain has an inflammatory component. “Inflammatory pain”, as described above, refers to pain which is produced as a symptom or a result of inflammation or an immune system disorder.

“Visceral pain”, as described above, refers to pain which is located in an internal organ.

“Mixed etiology” pain, as described above, refers to pain that contains both inflammatory and neuropathic components.

“Dual mechanism” pain, as described above, refers to pain that is amplified and maintained by both peripheral and central sensitization.

“Causalgia”, as described above, refers to a syndrome of sustained burning, allodynia, and hyperpathia after a traumatic nerve lesion, often combined with vasomotor and sudomotor dysfunction and later trophic changes.

“Central” pain, as described above, refers to pain initiated by a primary lesion or dysfunction in the central nervous system.

“Hyperesthesia”, as described above, refers to increased sensitivity to stimulation, excluding the special senses.

“Hyperpathia”, as described above, refers to a painful syndrome characterized by an abnormally painful reaction to a stimulus, especially a repetitive stimulus, as well as an increased threshold. It can occur with allodynia, hyperesthesia, hyperalgesia, or dysesthesia.

“Dysesthesia”, as described above, refers to an unpleasant abnormal sensation, whether spontaneous or evoked. Special cases of dysesthesia include hyperalgesia and allodynia,

“Hyperalgesia”, as described above, refers to an increased response to a stimulus that is normally painful. It reflects increased pain on suprathreshold stimulation.

“Allodynia”, as described above, refers to pain due to a stimulus that does not normally provoke pain.

The term “pain” includes pain resulting from dysfunction of the nervous system: organic pain states that share clinical features of neuropathic pain and possible common pathophysiology mechanisms, but are not initiated by an identifiable lesion in any part of the nervous system.

The term “Diabetic Peripheral Neuropathic Pain” (DPNP, also called diabetic neuropathy, DN or diabetic peripheral neuropathy) refers to chronic pain caused by neuropathy associated with diabetes mellitus. The classic presentation of DPNP is pain or tingling in the feet that can be described not only as “burning” or “shooting” but also as severe aching pain. Less commonly, patients can describe the pain as itching, tearing, or like a toothache. The pain can be accompanied by allodynia and hyperalgesia and an absence of symptoms, such as numbness.

The term “Post-Herpetic Neuralgia”, also called “Postherpetic Neuralgia” (PHN), is a painful condition affecting nerve fibers and skin. It is a complication of shingles, a second outbreak of the varicella zoster virus (VZV), which initially causes chickenpox.

The term “neuropathic cancer pain” refers to peripheral neuropathic pain as a result of cancer, and can be caused directly by infiltration or compression of a nerve by a tumor, or indirectly by cancer treatments such as radiation therapy and chemotherapy (chemotherapy-induced neuropathy).

The term “HIV/AIDS peripheral neuropathy” or “HIV/AIDS related neuropathy” refers to peripheral neuropathy caused by HIV/AIDS, such as acute or chronic inflammatory demyelinating neuropathy (AIDP and CIDP, respectively), as well as peripheral neuropathy resulting as a side effect of drugs used to treat HIV/AIDS.

The term “Phantom Limb Pain” refers to pain appearing to come from where an amputated limb used to be. Phantom limb pain can also occur in limbs following paralysis (e.g., following spinal cord injury). “Phantom Limb Pain” is usually chronic in nature.

The term “Trigeminal Neuralgia” (TN) refers to a disorder of the fifth cranial (trigeminal) nerve that causes episodes of intense, stabbing, electric-shock-like pain in the areas of the face where the branches of the nerve are distributed (lips, eyes, nose, scalp, forehead, upper jaw, and lower jaw). It is also known as the “suicide disease”.

The term “Complex Regional Pain Syndrome (CRPS),” formerly known as Reflex Sympathetic Dystrophy (RSD), is a chronic pain condition. The key symptom of CRPS is continuous, intense pain out of proportion to the severity of the injury, which gets worse rather than better over time. CRPS is divided into type 1, which includes conditions caused by tissue injury other than peripheral nerve, and type 2, in which the syndrome is provoked by major nerve injury, and is sometimes called causalgia.

The term “Fibromyalgia” refers to a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

The term “convulsion” refers to a CNS disorder and is used interchangeably with “seizure,” although there are many types of seizure, some of which have subtle or mild symptoms instead of convulsions. Seizures of all types can be caused by disorganized and sudden electrical activity in the brain. Convulsions are a rapid and uncontrollable shaking. During convulsions, the muscles contract and relax repeatedly.

### Compositions Including Stereoisomers

Certain compounds of the present invention possess asymmetric carbon atoms (optical centers) or double bonds; the racemates, diastereomers, geometric isomers and individual isomers are encompassed within the scope of the present invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

As used herein, the term “chiral”, “enantiomerically enriched” or “diastereomerically enriched” refers to a compound having an enantiomeric excess (ee) or a diastereomeric excess (de) of greater than about 50%, preferably greater than about 70% and more preferably greater than about 90%. In general, higher than about 90% enantiomeric or diastereomeric excess is particularly preferred, e.g., those compositions with greater than about 95%, greater than about 97% and greater than about 99% ee or de.

The terms “enantiomeric excess” and “diastereomeric excess” are used interchangeably herein. Compounds with a single stereocenter are referred to as being present in “enantiomeric excess”, those with at least two stereocenters are referred to as being present in “diastereomeric excess”.

The term “enantiomeric excess” is well known in the art and is defined as:

$${ee}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being enantiomerically pure. A compound which in the past might have been called 98% optically pure is now more precisely characterized by 96% ee. A 90% ee reflects the presence of 95% of one enantiomer and 5% of the other(s) in the material in question.

Hence, in one embodiment, the invention provides a composition including a first stereoisomer and at least one additional stereoisomer of a compound of the invention. The first stereoisomer can be present in a diastereomeric or enantiomeric excess of at least about 80%, preferably at least about 90% and more preferably at least about 95%. In a particularly preferred embodiment, the first stereoisomer is present in a diastereomeric or enantiomeric excess of at least about 96%, at least about 97%, at least about 98%, at least about 99% or at least about 99.5%. In another embodiment, the compound of the invention is enantiomerically or diastereomerically pure (diastereomeric or enantiomeric excess is about 100%). Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

### II. Introduction

The present invention relates to novel inhibitors of the enzyme D-amino acid oxidase.

The compounds of the invention are useful for treating or preventing any disease and/or condition, wherein modulation of D-serine levels, and/or its oxidative products, is effective in ameliorating symptoms. Inhibition of the enzyme can lead to increases in D-serine levels and a reduction in the formation of toxic D-serine oxidation products. Thus, the invention provides methods for the treatment or prevention of neurological disorders and methods of enhancing learning, memory and/or cognition. For example, compounds of the invention can be used for treating or preventing loss of memory and/or cognition associated with neurodegenerative diseases (e.g., Alzheimer's disease) and for preventing loss of neuronal function characteristic of neurodegenerative diseases. Further, methods are provided for the treatment or prevention of pain, ataxia and convulsion.

### III. Compositions

### A. Fused Heterocycles

The heterocyclic inhibitors of the invention are characterized by a variety of core-moieties. In an exemplary embodiment, the core-moiety includes a 5-membered, aromatic heterocyclic ring (first ring), such as a pyrrole, a furan, a thiophene or an imidazole fused to a second ring, wherein the second ring is a non-aromatic ring. In Formula (I), below, the second ring is marked with “(a)”. The second ring can optionally be fused to at least one additional ring (e.g., a cyclopropane ring). In one embodiment, second ring (a) is substituted or unsubstituted cyclopentene or substituted or unsubstituted cyclohexene. For the purpose of characterizing the second ring (a), a double bond is assumed to be located between the first and second ring. Two examples according to this embodiment are shown below:

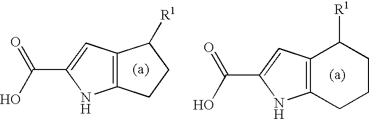

Other exemplary second rings (a) include substituted or unsubstituted cyclopentadienes, substituted or unsubstituted cyclohexadienes. In one embodiment, the second ring is substituted with a carbonyl group. Exemplary rings according to this embodiment include substituted or unsubstituted cyclopentenones, substituted or unsubstituted cyclopentadienones, substituted or unsubstituted cyclohexenones and substituted or unsubstituted cyclohexadienones.

In one embodiment, the compound of the invention has a structure according to Formula (I):

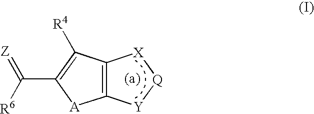

In one embodiment in Formula (I), Z is O. In another embodiment, Z is S. In yet another embodiment, A is NR⁷. In a further embodiment, A is S. In another embodiment, A is O.

In Formula (I), X, Q and Y are members independently selected from O, S, NR³, CR¹, —(CR¹R²)_{q}—, C═O, C═S, C═NR³ and C═CR⁴⁰R⁴¹, wherein each q is an integer independently selected from 1 and 2. In one embodiment, at least one of X, Q and Y is a member selected from CR¹, —(CR¹R²)_{q}—, C═O and C═S. In another embodiment, X, Q and Y are members independently selected from CR¹, —(CR¹R²)_{q}—, C═O and C═S and C═CR⁴⁰R⁴¹. In yet another example, at least one member selected from X and Y is CH₂, CHF or CF₂ and the other member is CHR¹, wherein R¹ is other than H. In a further example, at least one member selected from X and Y is CH₂ and the other member is CHR¹, wherein R¹ is other than H. In another example, Q is a member selected from —(CH₂)ₙ—, CHF, CF₂, CHCl, CHOH, CHMe, C═O and C═S, wherein r is an integer selected from 1 and 2. X and Q are optionally joined to form a 3- to 7-membered ring. Y and Q are optionally joined to form a 3- to 7-membered ring. X and Y, together with the atoms to which they are attached, are optionally joined to form a 3- to 7-membered ring (e.g., forming a bridged bicyclic substructure).

In Formula (I), the ring, which includes X, Q and Y [ring (a)] is a non-aromatic ring and can be a 5-, 6-, 7- or 8-membered ring. In one embodiment, ring (a) is a 5-membered ring. Exemplary 5-membered rings according to this embodiment include substituted or unsubstituted cyclopentene, substituted or unsubstituted cyclopentadienes, substituted or unsubstituted dihydrofuranes, substituted or unsubstituted dihydrothiophenes, substituted or unsubstituted dihydropyrroles, substituted or unsubstituted dihydroimidazoles and substituted or unsubstituted 3H-pyrazoles. When ring (a) is a 5-membered ring and includes a double bond between X and Q or between Y and Q, then ring (a) does preferably not include a heteroatom.

In another embodiment, ring (a) is a six-membered ring. Exemplary six-membered rings according to this embodiment, include substituted or unsubstituted cyclohexene, substituted or unsubstituted cyclohexadienes, substituted or unsubstituted dihydropyranes, substituted or unsubstituted tetrahydropyridines, substituted or unsubstituted dihydropyridines, substituted or unsubstituted dihydrothiopyranes, substituted or unsubstituted 1,2 thiazines, substituted or unsubstituted 1,3, thiazines, substituted or unsubstituted dihydropyrimidines and substituted or unsubstituted dihydropyrazines.

In Formula (I), each R³ and each R⁷ are members independently selected from H, OR¹², acyl, NR¹²R¹³, SO₂R¹³, SOR¹³, substituted or unsubstituted alkyl, substituted or unsubstituted heteroalkyl, substituted or unsubstituted aryl, substituted or unsubstituted heteroaryl and substituted or unsubstituted heterocycloalkyl, wherein R¹² and R¹³ are members independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

In Formula (I), each R¹, each R², each R⁴⁰, each R⁴¹ and R⁴ are members independently selected from H, halogen (e.g., F, Cl, Br, I), CN, halogen-substituted alkyl (e.g., CF₃), acyl, C(O)OR¹⁴, C(O)NR¹⁴R¹⁵, OR¹⁴, S(O)₂OR¹⁴, S(O)ₚR¹⁴, SO₂NR¹⁴R¹⁵, NR¹⁴R¹⁵, NR¹⁴C(O)R¹⁵, NR¹⁴S(O)₂R¹⁵, substituted or unsubstituted alkyl, substituted or unsubstituted heteroalkyl, substituted or unsubstituted aryl, substituted or unsubstituted heteroaryl and substituted or unsubstituted heterocycloalkyl, wherein p is an integer selected from 0 to 2. R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R¹⁴ and R¹⁵, together with the atoms to which they are attached, are optionally joined to form a 5- to 7-membered ring.

R¹ and R², together with the atoms to which they are attached, are optionally joined to form a 3- to 7-membered ring. In one example, R¹ and R² are members independently selected from substituted or unsubstituted alkyl (e.g., methyl, ethyl, propyl, butyl, pentyl or hexyl), substituted or unsubstituted arylalkyl (e.g., phenyl-alkyl), substituted or unsubstituted heteroarylalkyl (e.g., pyridinyl-alkyl), substituted or unsubstituted cycloalkyl-alkyl and substituted or unsubstituted heterocycloalkyl-alkyl. In one embodiment, at least one of R¹, R², R³ and R⁴ is other than H. In another embodiment, at least one of R¹ and R² is other than H. In an exemplary embodiment CR¹R² is CF₂.

In one example, R⁴ represents a small substituent, such as H, halogen (e.g., F, Cl, Br, I), CN, CF₃, OH, OMe, OEt, methyl, ethyl and propyl. In another example, R⁴ is H, F, Cl, CN or Me. In yet another example, R⁴ is H or F.

In Formula (I), R⁶ is a member selected from OR⁸, O⁻X⁺, NR⁹R¹⁰, NR⁹NR^{9′}R¹⁰, NR⁹OR¹⁰, NR⁹SO₂R¹¹, substituted or unsubstituted alkyl, substituted or unsubstituted heteroalkyl, substituted or unsubstituted aryl, substituted or unsubstituted heteroaryl and substituted or unsubstituted heterocycloalkyl, wherein X⁺ is an organic or inorganic cation (e.g., Na⁺, NH₄⁺, K⁺ or another pharmaceutically acceptable salt forms). In one example, R⁶ is a member selected from OR⁸, O⁻X⁺, NR⁹R¹⁰, NR⁹NR^{9′}R¹⁰, NR⁹OR¹⁰ and NR⁹SO₂R¹¹. In another example, R⁶ is a member selected from OR⁸ and O⁻X⁺. R⁶ and R⁷, together with the atoms to which they are attached, are optionally joined to form a 5- to 7-membered ring. R⁶ and R⁴, together with the atoms to which they are attached, are optionally joined to form a 5- to 7-membered ring.

In Formula (I), R⁸ is a member selected from H, a single negative charge, substituted or unsubstituted alkyl, substituted or unsubstituted heteroalkyl, substituted or unsubstituted aryl, substituted or unsubstituted heteroaryl and substituted or unsubstituted heterocycloalkyl. R⁹, R^{9′} and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R¹¹ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. At least two of R⁸, R⁹, R^{9′}, R¹⁰ and R¹¹, together with the atoms to which they are attached, are optionally joined to form a 5- to 7-membered ring.

In one embodiment, wherein R⁴ is H or CH₃, A is NR⁷, and Z is O, X, Q and Y are preferably not all CH₂. In another embodiment, wherein R⁴ is H or CH₃, A is NR⁷, Z is O and one member selected from X, Q and Y is CH₂CH₂, the other two members are preferably not both CH₂. For example, when R⁴ is H, A is NR⁷ and Z is O, ring (a) is preferably not unsubstituted cyclohexene or unsubstituted cyclopentene.

In one embodiment, the compound of the invention has a structure according to one of the following Formulae:

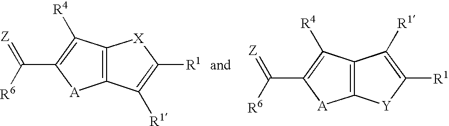

wherein X and Y are members selected from —(CR¹R²)_{q}—, C═CR⁴⁰R⁴¹, C═O, C═S and C═NR³, wherein q is selected from 1 and 2.

In another embodiment, the compound of the invention has a structure according to one of the following Formulae:

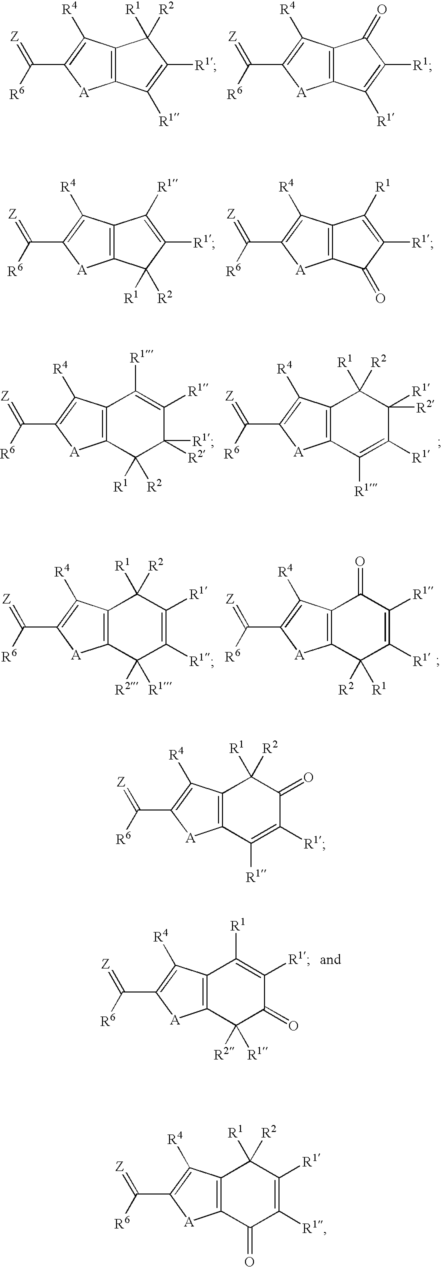

wherein Z, A, R⁶, R⁴, R¹ and R² are defined as for Formula (I), above. R^{1′}, R^{1″}, R^{1′″} are defined as R¹. R^{2′}, R^{2″}, R^{2′″} are defined as R². In one example, in the above structures, R⁴ is H. In another example, in the above structures, A is NH. In yet another example, in the above structures A is O. In a further example, R⁶ is OR⁸, wherein R⁸ is defined as herein above.

In yet another embodiment, the invention provides a compound having a structure according to Formula (II):

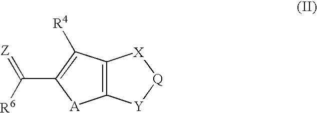

wherein Z, A, R⁴ and R⁶ are defined as for Formula (I), above. Exemplary embodiments listed for Formula (I) equally apply to compounds of Formula (II). In one example, in Formula (II), X, Q and Y are members independently selected from O, S, NR³, —(CR¹R²)_{q}—, C═CR⁴⁰R⁴¹, C═O, C═S and C═NR³, wherein q, R¹, R², R³, R⁴⁰ and R⁴¹ are defined as above for Formula (I). In one example, X, Q and Y are members independently selected from —(CR¹R²)_{q}—, C═CR⁴⁰R⁴¹, C═O and C═S. In one example, at least one of R¹, R², R³ and R⁴ in Formula (II) is other than H. In another example, at least one of R¹ and R² in Formula (II) is other than H.

In a further embodiment, the compound of the invention has a structure according to one of the following Formulae:

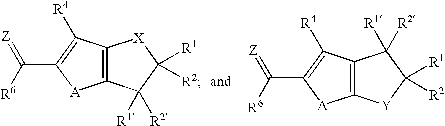

wherein Z, A, R⁴ and R⁶ are defined as for Formula (I), above, and X and Y are members independently selected from O, S, NR³, —(CR¹R²)_{q}—, C═CR⁴⁰R⁴¹, C═O, C═S and C═NR³. In one example according to this embodiment, X, Q and Y are members independently selected from —(CR¹R²)_{q}—, C═CR⁴⁰R⁴¹, C═O, C═S and C═NR³.

In yet another embodiment, the compound of the invention has a structure according to one of the following Formulae:

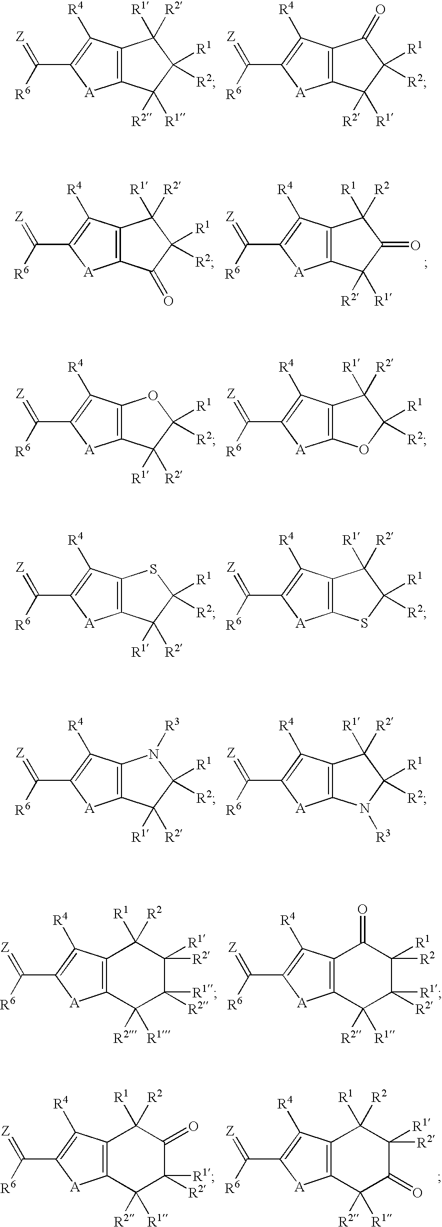

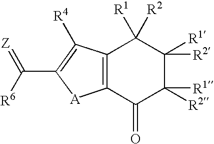

wherein Z, A, R⁶, R⁴, R¹ and R² are defined as for Formula (I), above. R^{1′}, R^{1″}, R^{1′″} are defined as R¹. R^{2′}, R^{2″}, R^{2′″} are defined as R². In one example, in the above structures, R⁴ is H. In another example, in the above structures, A is NH. In yet another example, in the above structures A is O. In a further example, R⁶ is O⁻X⁺ or OR⁸, wherein R⁸ and X⁺ are defined as herein above. For example, R⁸ is a member selected from H and a single negative charge.

In another embodiment, at least one of X, Q and Y includes F. In one example, at least one of X, Q and Y is CHF or CF₂. Exemplary compounds according to this example have a formula, which is a member selected from:

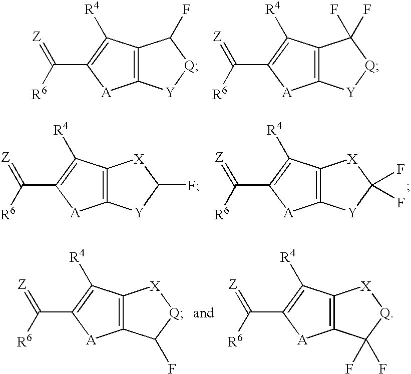

Other exemplary compounds according to this embodiment include:

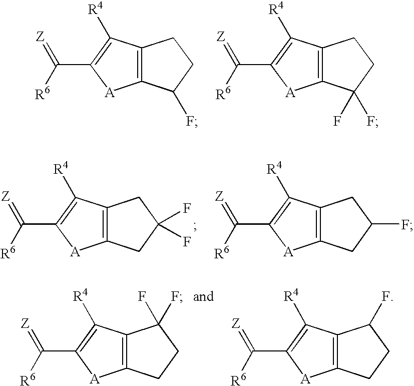

In another exemplary embodiment, the compound of the invention has a structure selected from:

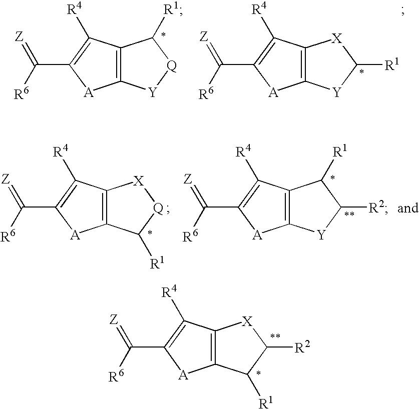

wherein Z, A, R⁴, R⁶, R¹ and R² are defined as for Formula (I). In the above structures, each stereocenter marked with an asterix “*” or “**” is independently either racemic or defined. In one example, the stereocenter marked with “*” has (R)-configuration. In another example, the stereocenter marked with “*” has (S)-configuration. R¹ and R², together with the atoms to which they are attached, are optionally joined to form a 3- to 7-membered ring.

In one embodiment R¹ and R² are joined to form a substituted or unsubstituted cyclopropane ring. Exemplary compounds according to this embodiment have a structure selected from the following formulae:

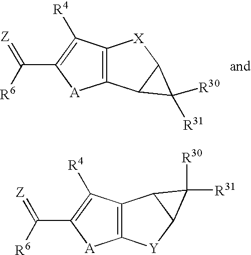

wherein R³⁰ and R³¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

Exemplary compounds include:

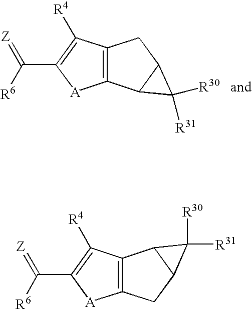

In one example, according to the above embodiments, at least one of R³⁰ and R³¹ has the formula:

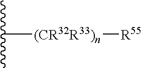

wherein n is an integer from 0 to 5. R⁵⁵ is a substituted or unsubstituted aromatic or non-aromatic ring. Exemplary embodiments described herein below for R⁵⁰ equally apply to R⁵⁵. In one example, R⁵⁵ is a member selected from substituted or unsubstituted aryl, substituted or unsubstituted heteroaryl, substituted or unsubstituted cycloalkyl and substituted or unsubstituted heterocycloalkyl. In one example, each R³² and each R³³ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In another example, each R³² and each R³³ is a member independently selected from H, substituted or unsubstituted alkyl and substituted or unsubstituted heteroalkyl. In one example, example, n is 1, 2 or 3. In another example, (CR³²R³³)ₙ is a member selected from unsubstituted methylene (CH₂), unsubstituted ethylene (CH₂CH₂) and unsubstituted n-propylene (CH₂CH₂CH₂). R³² and R³³, together with the carbon atom to which they are attached, are optionally joined to form a 3- to 7-membered ring, which is optionally fused to R⁵⁵. In one example, the ring formed by R³² and R³³ is a member selected from substituted or unsubstituted cycloalkyl and substituted or unsubstituted heterocycloalkyl.

In another example, according to the above embodiments, R⁵⁵ is an aromatic ring. In one example according to this embodiment, at least one of R³⁰ and R³¹ has the formula:

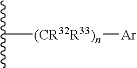

wherein Ar is a member selected from substituted or unsubstituted aryl and substituted or unsubstituted heteroaryl.

In yet another embodiment, the compound of the invention has the structure:

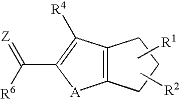

wherein Z, A, R⁴, R⁶, R¹ and R² are defined as herein above for Formula (I). In a preferred embodiment, at least one of R¹, R² and R⁴ is other than H. In another preferred embodiment, at least one of R¹ and R² is other than H.

Exemplary compounds according to the above embodiment have a structure according to one of the following formulae:

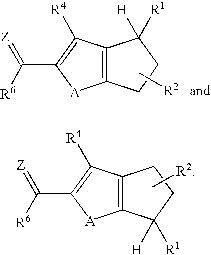

In one exemplary embodiment, the compound of the invention is chiral. Exemplary compounds according to this embodiment have a structure selected from:

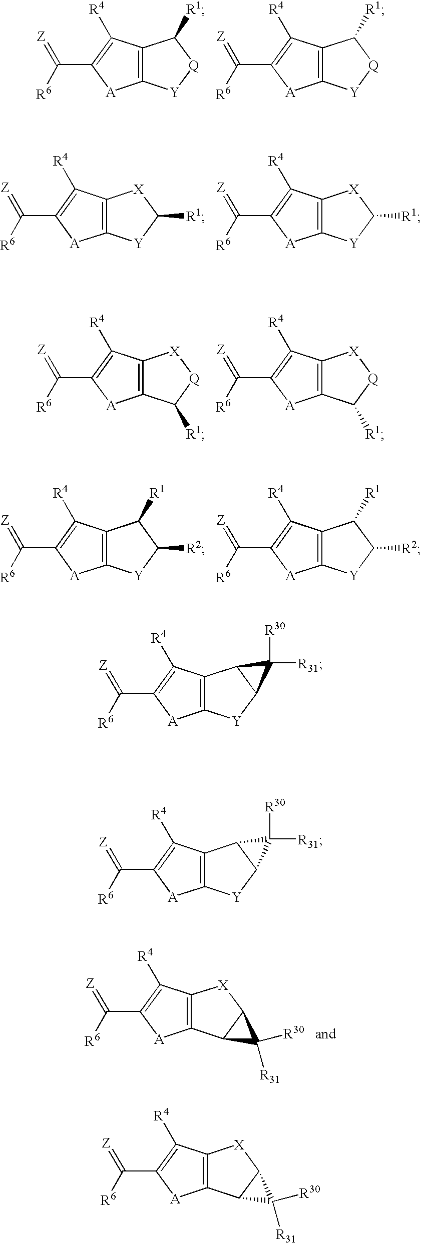

wherein Z, A, R⁴, R⁶, R¹ and R² are defined as herein above for Formula (I), with the proviso that R¹ is other than H. In the above structures, absolute stereochemistry is shown.

Exemplary compounds according to this embodiment include:

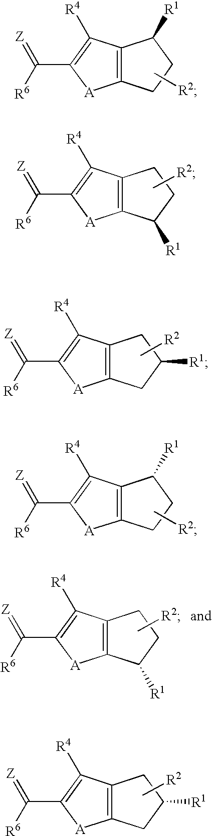

wherein absolute stereochemistry is shown. A person of skill in the art will understand that when R¹ and R² are the same and are both bound to the same carbon atom, the resulting compound is not chiral with respect to the shown stereocenter.

In another embodiment, the compound has a structure according to Formula (IVa), Formula (IVb), Formula (Va) or Formula (Vb):

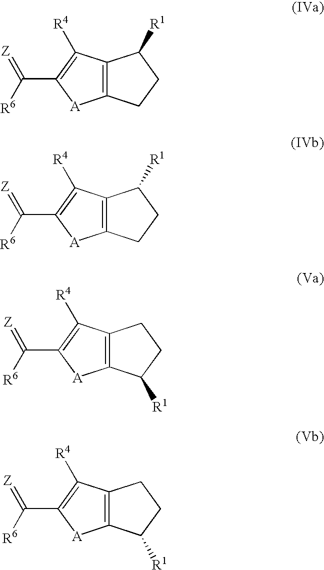

wherein absolute stereochemistry is shown. In the above structures, R¹ is defined as above with the proviso that R¹ is other than H. In one example, in Formula (IVa), Formula (IVb), Formula (Va) or Formula (Vb), R¹ is a member selected from C₁-C₁₀ substituted or unsubstituted alkyl. In another example, R¹ is a member selected from substituted or unsubstituted methyl, ethyl, n-propyl, iso-propyl, n-butyl and iso-butyl. In yet another example, R¹ is aryl-substituted or heteroaryl-substituted methyl, ethyl or propyl. In a particular example, R¹ is phenyl-substituted methyl, ethyl or propyl. In yet another example, in the above structures R⁴ is H or F.

In yet another exemplary embodiment, the compound has a structure according to the following formulae:

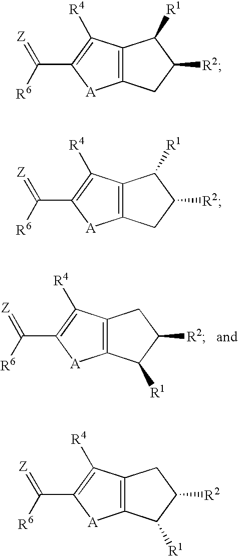

wherein absolute stereochemistry is shown. In the above structures, R¹ and R² are other than H. R¹ and R², together with the atoms to which they are attached, are optionally joined to form a 3- to 7-membered ring. In one example, R¹ and R² are joined to form a substituted or unsubstituted cyclopropane ring.

In one example according to any of the embodiments outlined herein above, at least one of R¹, R² and R³ includes a ring or a fused ring system. In one embodiment, at least one of R¹, R² and R³ has the formula:

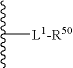

wherein R⁵⁰ is selected from a substituted or unsubstituted aromatic or non-aromatic ring. In one example, R⁵⁰ is a member selected from substituted or unsubstituted aryl, substituted or unsubstituted heteroaryl, substituted or unsubstituted cycloalkyl and substituted or unsubstituted heterocycloalkyl. Exemplary aromatic rings R⁵⁰ include substituted or unsubstituted phenyl, substituted or unsubstituted pyridines, substituted or unsubstituted pyrimidines, substituted or unsubstituted furanes, substituted or unsubstituted oxazoles, substituted or unsubstituted isoxazoles, substituted or unsubstituted thiazoles and substituted or unsubstituted isothiazoles. Exemplary non-aromatic rings R⁵⁰ include substituted or unsubstituted cyclohexanes, substituted or unsubstituted tetrahydro-2H-pyranes, substituted or unsubstituted morpholines, substituted or unsubstituted piperidines, substituted or unsubstituted N-alkyl-piperazines, substituted or unsubstituted cyclopentanes, substituted or unsubstituted pyrrolidines and substituted or unsubstituted oxazolidines.

L¹ is a linker moiety, which is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. In one example, L¹ is a member selected from substituted or unsubstituted alkyl and substituted or unsubstituted heteroalkyl. In another example, L¹ is a substituted or unsubstituted alkyl chain, wherein one or more carbon atoms are optionally replaced with a heteroatom or a functional group, forming e.g., ether, thioether, amines, amides, sulfonamides, sulfones carbonates, ureas or the like. In another example, L¹ is unsubstituted methylene, ethyl, n-propylene, n-butylene or n-propylene, optionally linked to the remainder of the molecule or the ring R⁵⁰ via a heteroatom or a functional group, e.g., via an ether, amine, carbonamide or sulfonamide group.

In another example according to any of the embodiments herein above, at least one of R¹, R² and R³ has a formula, which is a member selected from:

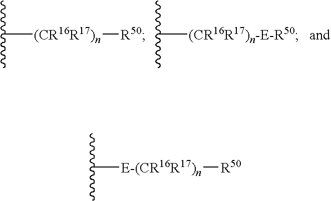

wherein n is an integer from 0 to 5. E is a heteroatom or a functional group, such as ether, thioether, carbonamide, sulfonamide, carbonate, urea and the like. In one example, E is a member selected from O, S, NR⁴³, C(O)NR⁴³, NR⁴³C(O), S(O)₂NR⁴³ and NR⁴³S(O)₂, wherein R⁴³ is a member selected from H, substituted or unsubstituted alkyl, substituted or unsubstituted heteroalkyl, substituted or unsubstituted aryl, substituted or unsubstituted heteroaryl and substituted or unsubstituted heterocycloalkyl. Each R¹⁶ and each R¹⁷ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In one example, example, n is 1, 2 or 3. In a further example, (CR¹⁶R¹⁷)ₙ is a member selected from unsubstituted methylene (CH₂), unsubstituted ethylene (CH₂CH₂) and unsubstituted n-propylene (CH₂CH₂CH₂). In one example, R¹⁶ and R¹⁷ are both H. At least two of R¹⁶ and R¹⁷, together with the carbon atom to which they are attached, are optionally joined to form a 3- to 7-membered ring. In an exemplary embodiment, the ring is a member selected from substituted or unsubstituted cycloalkyl and substituted or unsubstituted heterocycloalkyl, and is optionally fused to R⁵⁰. In one example according to this embodiment, R⁵⁰ is selected from substituted or unsubstituted aryl and substituted or unsubstituted heteroaryl.

In yet another example according to the above embodiments, R⁵⁰ represents an aromatic ring or a fused ring system including an aromatic ring. In one embodiment, at least one of R¹, R² and R³ has the formula:

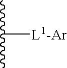

wherein Ar is a member selected from substituted or unsubstituted aryl, substituted or unsubstituted heteroaryl and a fused ring system, wherein the fused ring system includes at least one aromatic ring. L¹ is defined herein above. In one example according to any of the above embodiments, Q is CHR¹ or CFR¹, wherein R¹ represents a small substituent, such as H, F, Cl or methyl and one of X and Y is CHR² or NR³, wherein a member selected from R² and R³ includes the aromatic moiety.

In an exemplary embodiment, Ar is a phenyl ring and has the formula:

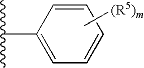

wherein m is an integer from 0 to 5. Each R⁵ is a member independently selected from aryl group substituents. In an exemplary embodiment, each R⁵ is a member independently selected from H, halogen, CN, halogen substituted alkyl (e.g., CF₃), hydroxy, alkoxy (e.g., methoxy and ethoxy), acyl (e.g., acetyl), CO₂R¹⁸, OC(O)R¹⁸, NR¹⁸R¹⁹, C(O)NR¹⁸R¹⁹, NR¹⁸C(O)R²⁰, NR¹⁸SO₂R²⁰, S(O)₂R²⁰, S(O)R²⁰, substituted or unsubstituted alkyl (e.g., methyl, ethyl, propyl or butyl), substituted or unsubstituted heteroalkyl, substituted or unsubstituted aryl, substituted or unsubstituted heteroaryl and substituted or unsubstituted heterocycloalkyl, wherein adjacent R⁵ are optionally joined to form a ring, such as substituted or unsubstituted cycloalkyl, substituted or unsubstituted heterocycloalkyl, substituted or unsubstituted aryl and substituted or unsubstituted heteroaryl.

R¹⁸ and R¹⁹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R²⁰ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. R¹⁸ and a member selected from R¹⁹ and R²⁰, together with the atoms to which they are attached, are optionally joined to form a 5- to 7-membered ring.

Exemplary compounds according to the above embodiments include:

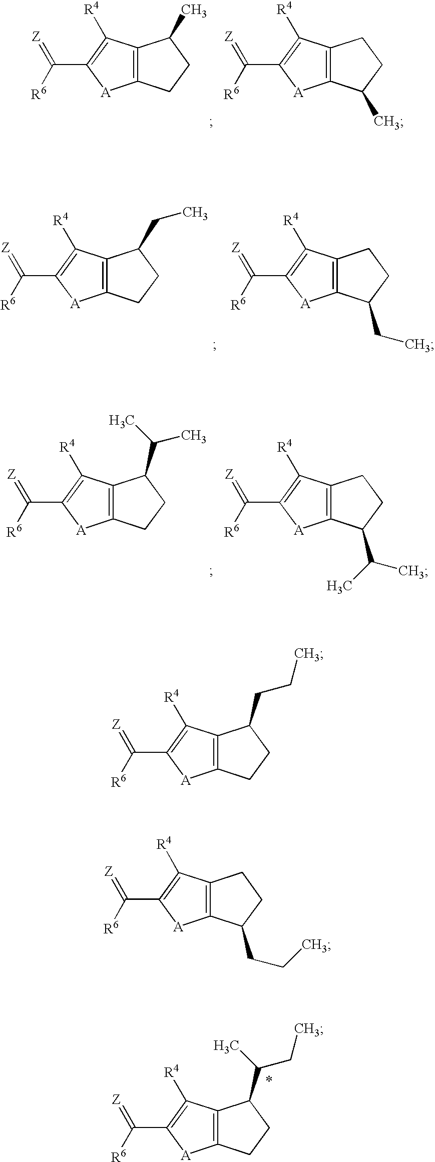

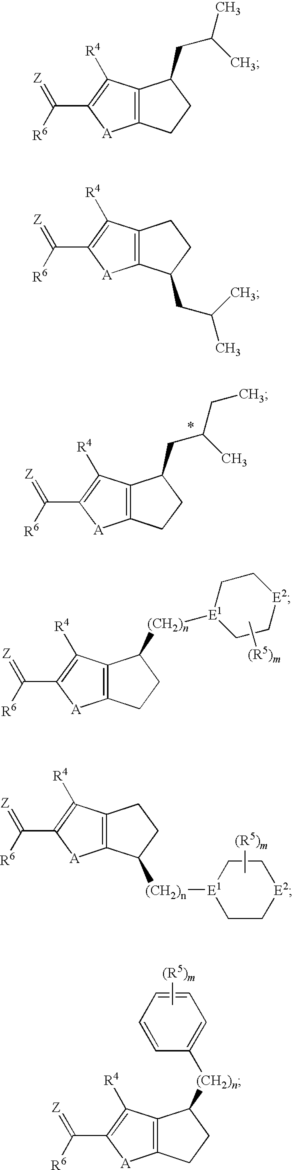

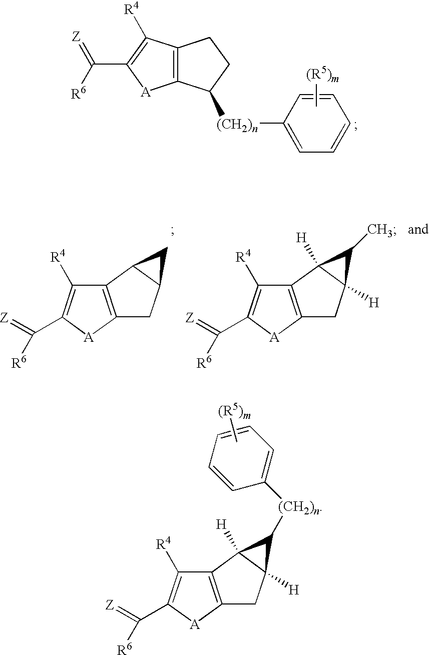

wherein m is an integer selected from 0 to 5 and n is an integer selected from 0 to 5. In one example, n is 1. In another example, n is 2. E¹ is selected from CH and N. E² is a member selected from CH₂, O and NR⁵¹, wherein R⁵¹ is a member selected from substituted or unsubstituted alkyl, e.g., methyl or ethyl. In one example, A is NH. In another example, A is S. In yet another example, A is O. In a further example, Z is O. In a particular example, Z is O, and A is NH or S and R⁶ is OR⁸ or O⁻X⁺.

In one example, according to any of the above embodiments, e.g., in Formulae (I) to (V), the compound of the invention is a pyrrole analog, in which A is NR⁷. In one example the compound of the invention has a structure according to Formula (III):

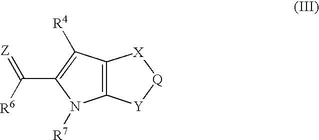

wherein Z, R⁴, R⁶ and R⁷ are defined as for Formula (I), above. Exemplary embodiments outlined herein above for Formulae (I) and (II) equally apply to Formula (III). In one embodiment in Formula (III), R⁴ is H. In another embodiment, Z is O. In yet another embodiment, R⁶ is OR⁵ or O⁻X⁺. In one example in Formula (III), X, Q and Y are members independently selected from O, S, NR³, CR¹R², C═CR⁴⁰R⁴¹, C═O, C═S and C═NR³, wherein R¹, R², R³, R⁴⁰ and R⁴¹ are defined as for Formula (I), above. In one example, in Formula (III), X, Q and Y are members independently selected from CR¹R², C═CR⁴⁰R⁴¹, C═O and C═S. In one example according to any of the above embodiments, R⁷ is H. In another example according to any of the above embodiments, Z is O.

In one example according to any of the above embodiments, R⁷ is H. Exemplary fused pyrroles have the structure:

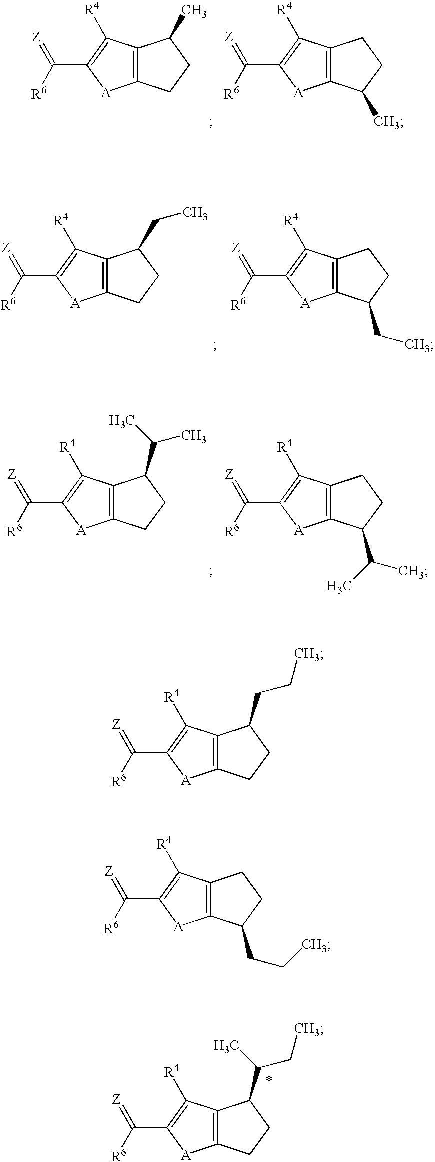

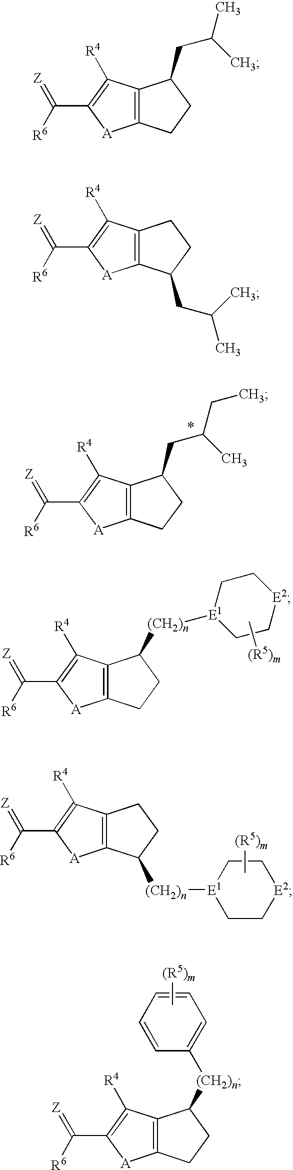

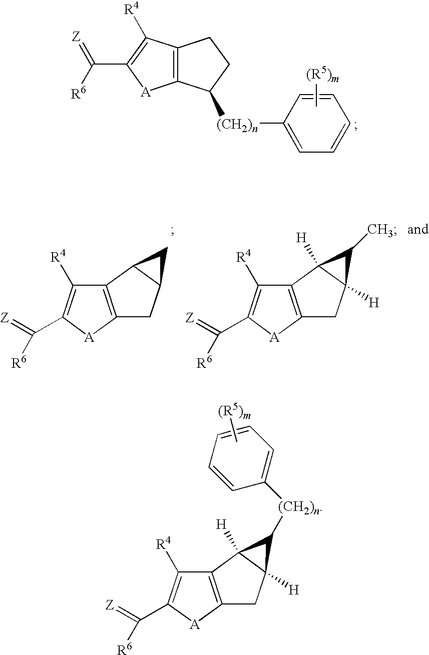

wherein absolute stereochemistry is shown. In the above structures, m and n are integers independently selected from 0 to 5. In one example, n is 1. In another example, n is 2. R⁵ is defined as above. E¹ is selected from CH and N. E² is a member selected from CH₂, O and NR⁵¹, wherein R⁵¹ is a member selected from substituted or unsubstituted alkyl e.g., methyl or ethyl. In a preferred embodiment in the above structures, Z is O.

Other exemplary compounds include:

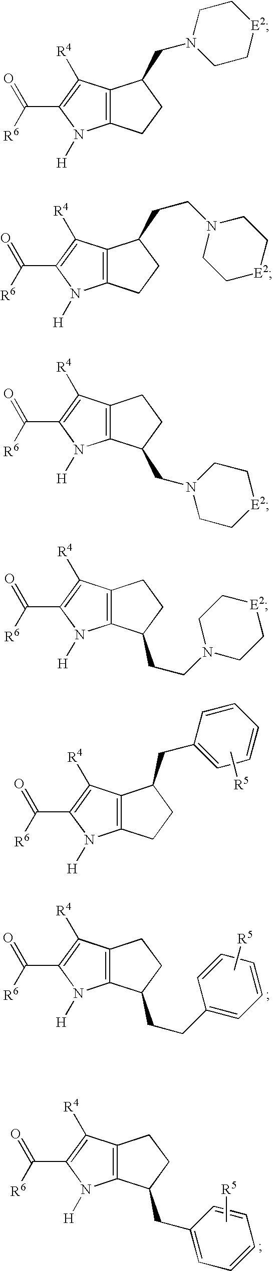

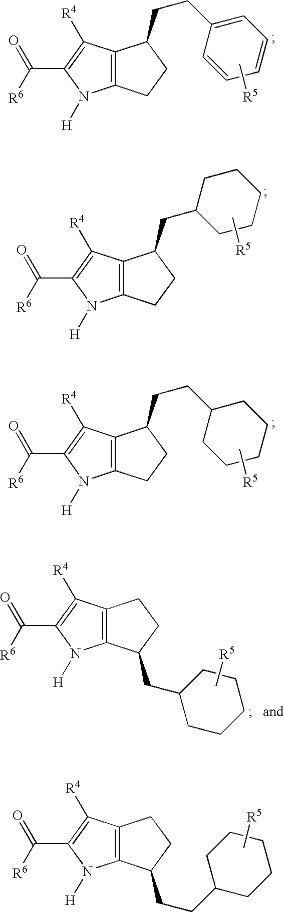

wherein absolute stereochemistry is shown. In one example, according to the above structures, R⁶ is OR⁸ or O⁻X⁺. In a preferred embodiment, R⁸ is a member selected from H and a single negative charge. X⁺ is a cation (salt counterion), such as Na⁺, K⁺ or another pharmaceutically acceptable organic or inorganic salt. In another example according to the above structures, R⁴ is selected from H and F.

In one example according to any of the above embodiments, e.g., in Formulae (I) to (V), Z is O. In another example according to any of the above embodiments, e.g. in Formulae (I) to (V), R⁶ is OR⁸ or O⁻X⁺. In a preferred embodiment, R⁸ is a member selected from H and a single negative charge. X⁺ is a cation (salt counterion), for example, Na⁺, K⁺ or another pharmaceutically acceptable organic or inorganic cation. In yet another example, according to any of the above embodiments, R⁴ is H or F.

In yet another example according to any of the above embodiments, Z is O and R⁶ is OR⁸ or O⁻X⁺, wherein R⁸ is a member selected from H and a single negative charge.

Exemplary compounds according to this embodiment include:

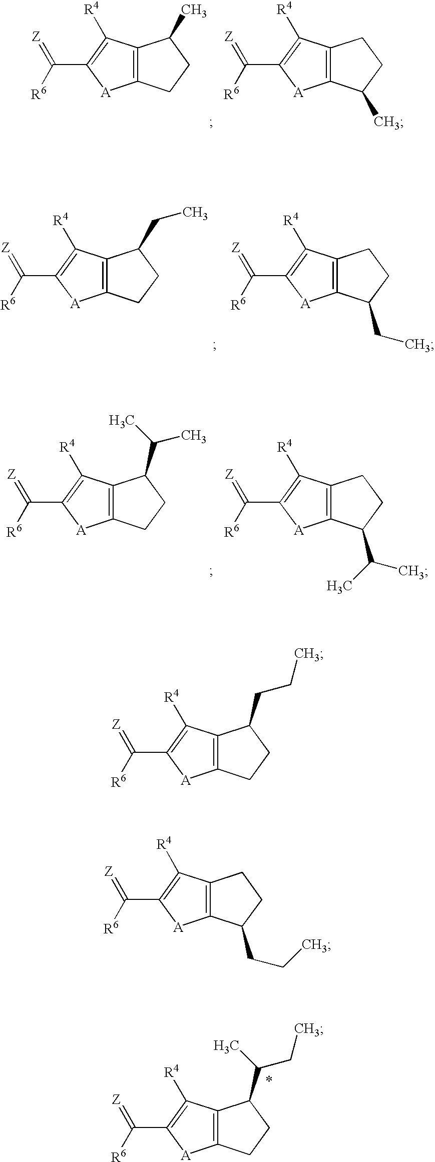

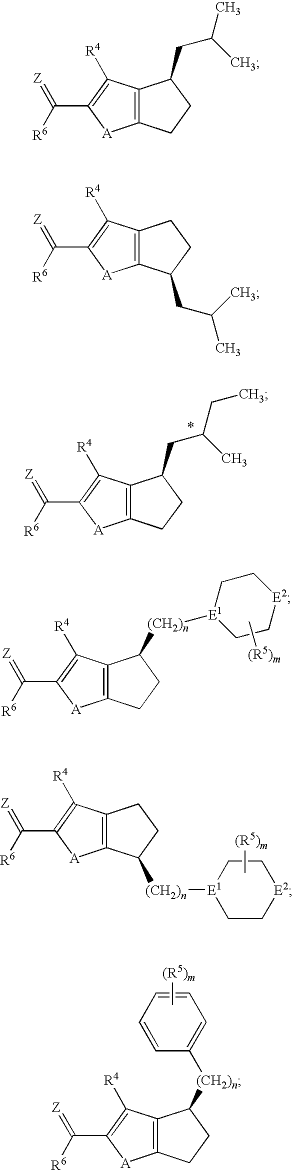

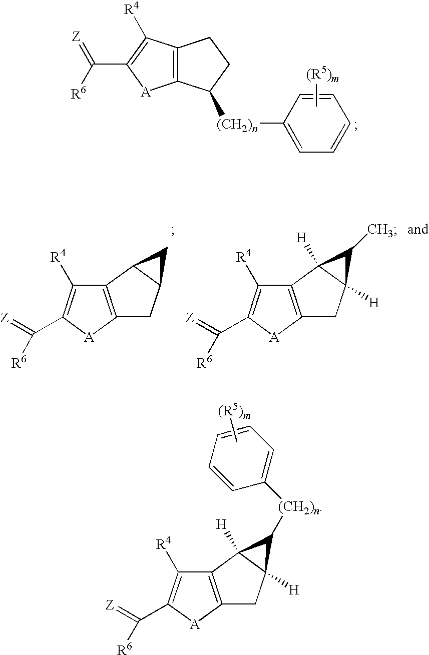

wherein absolute stereochemistry is shown. In one example, according to the above structures, R⁴ is H.

Other exemplary compounds of the invention include:

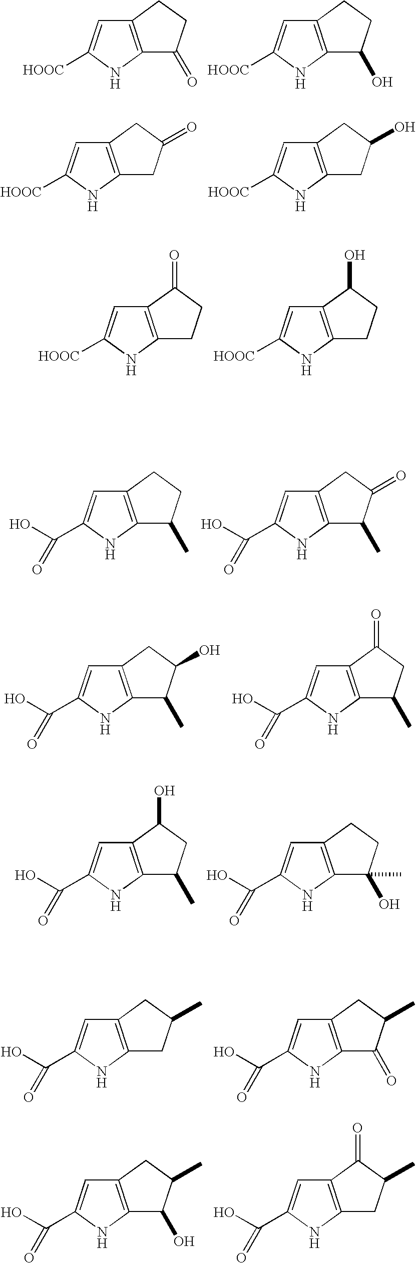

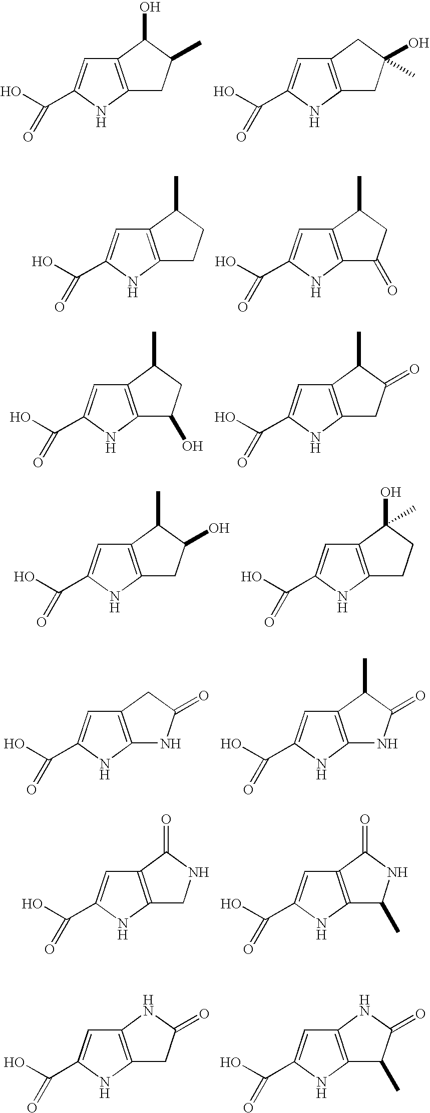

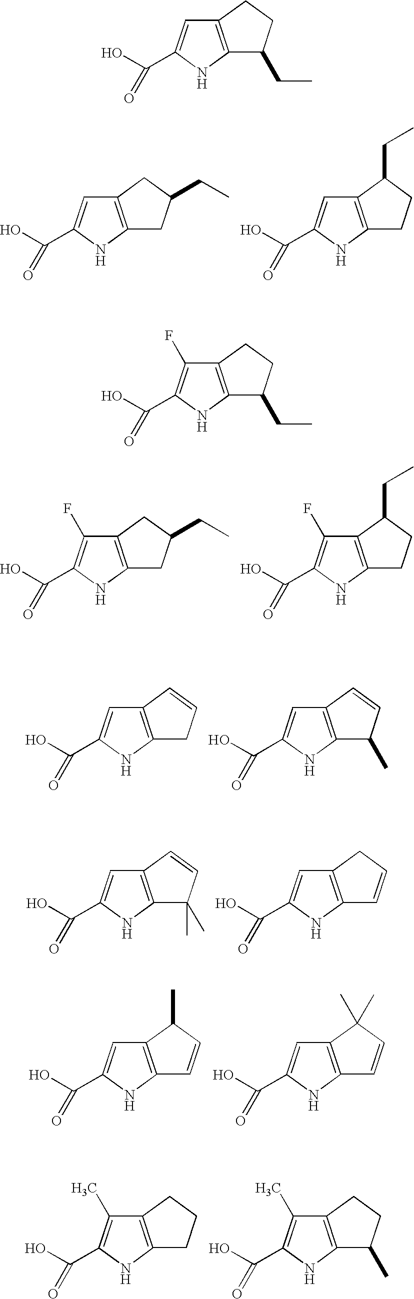

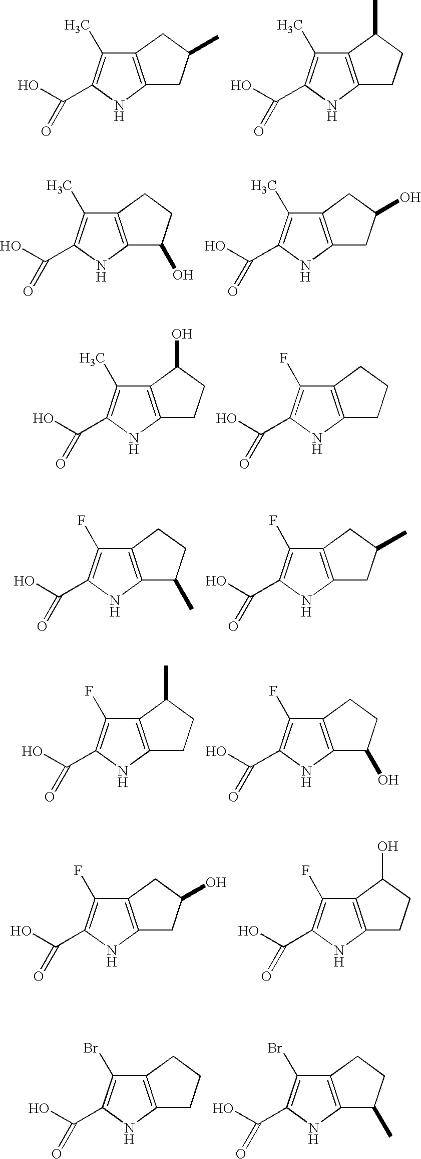

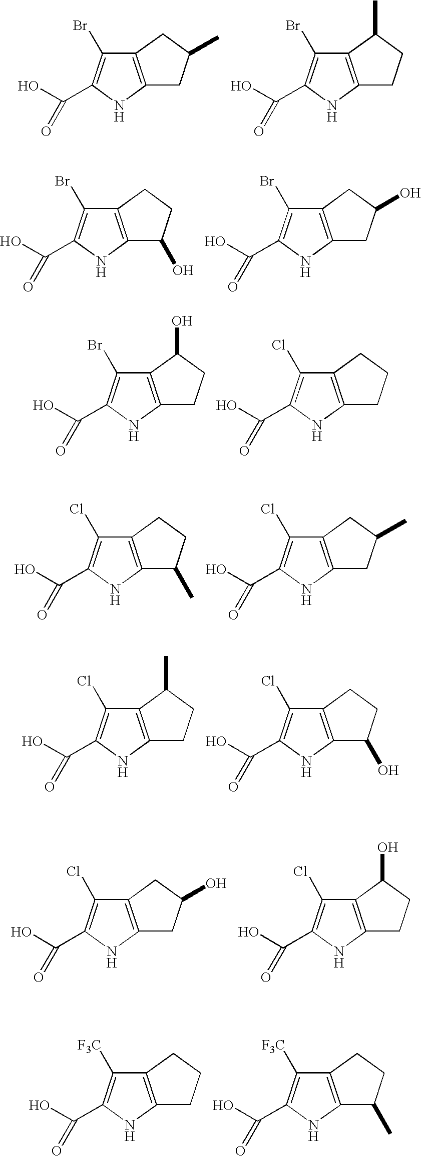

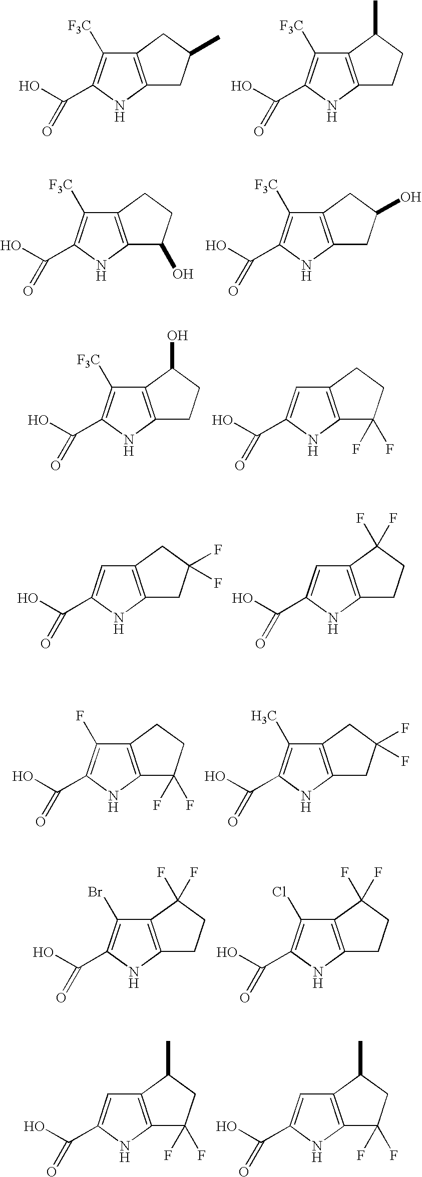

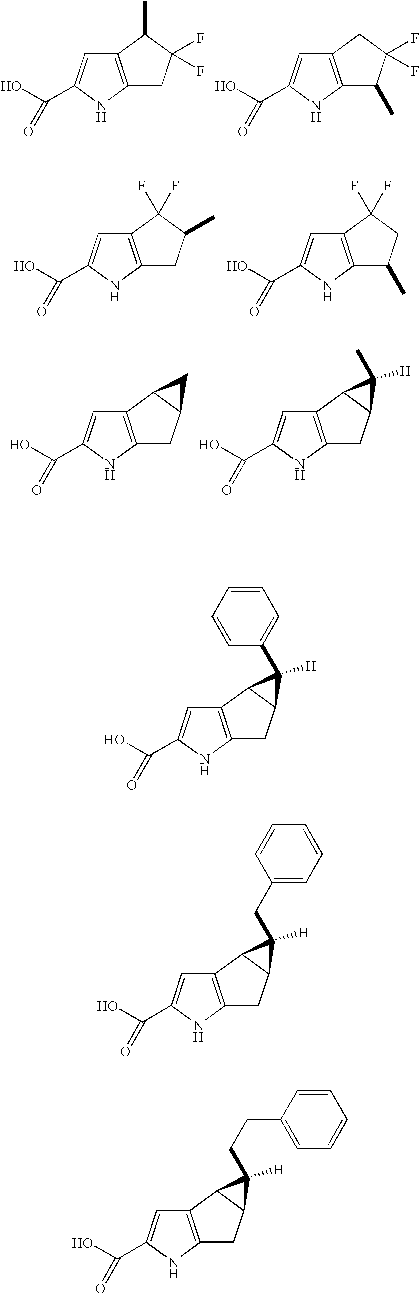

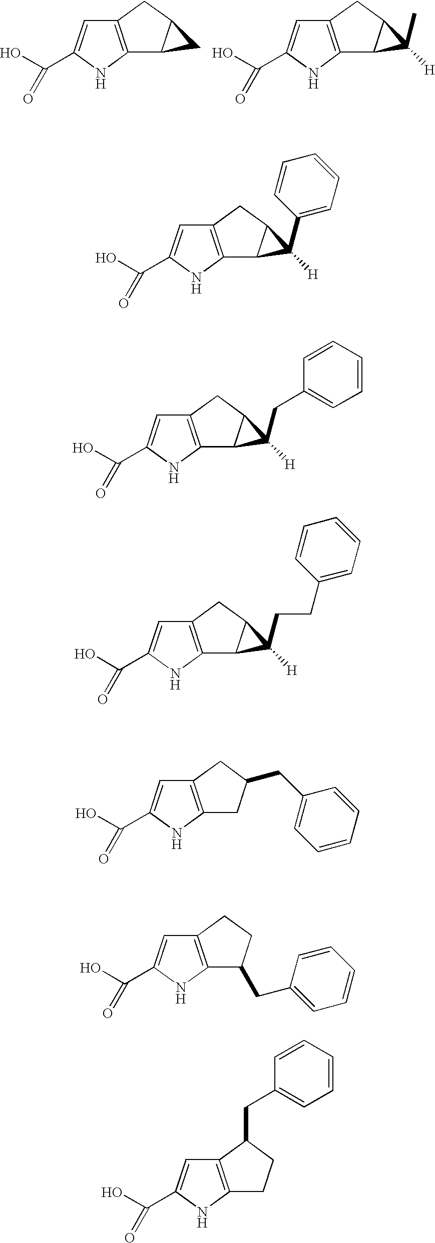

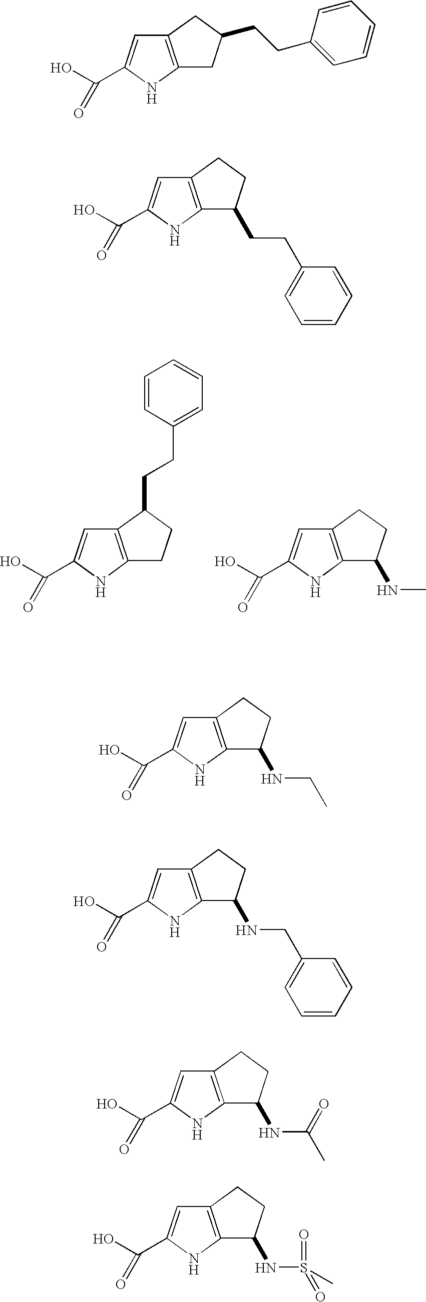

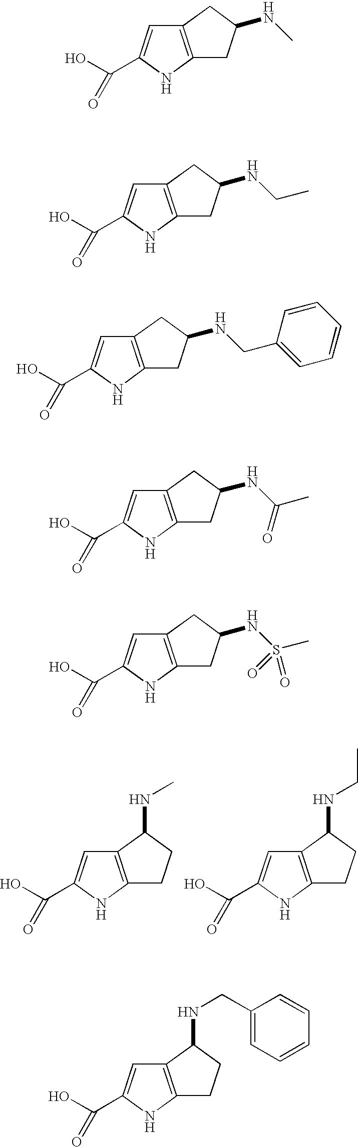

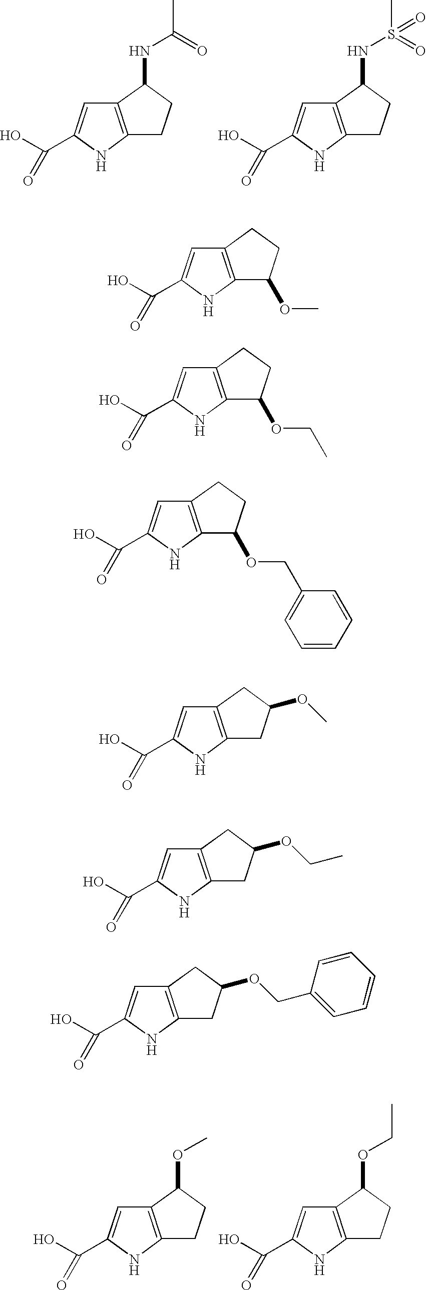

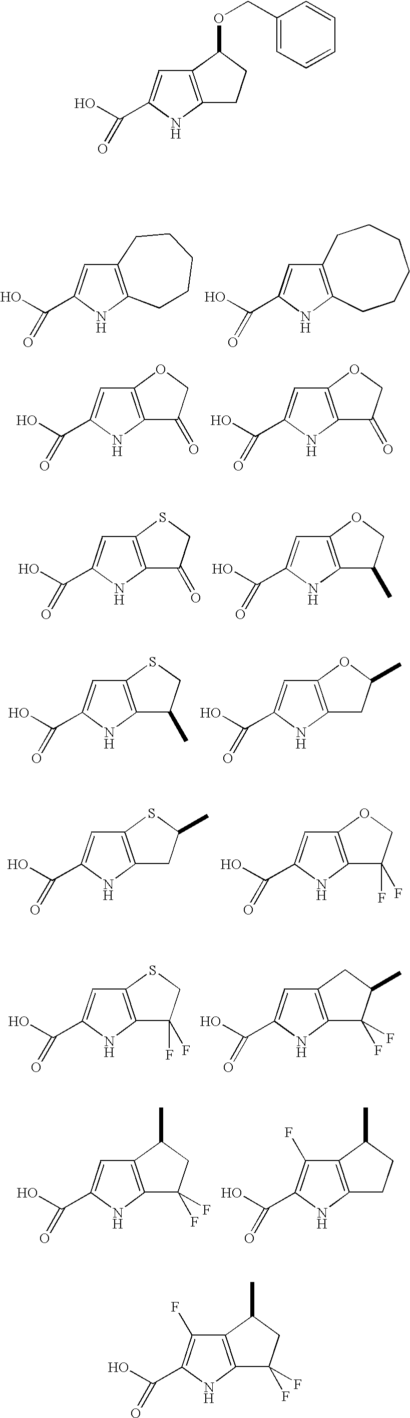

wherein relative stereochemistry is shown. A person of skill in the art will appreciate that the carboxylic acid group of the above compounds can optionally be deprotonated or the compounds can be present as a salt form, wherein the hydrogen of the carboxylic acid group is replaced with a cation (salt counterion).

In another exemplary embodiment, X and Y, together with the atoms to which they are attached, are optionally joined to form a 5- to 7-membered ring. In this case a bicyclic substructure is formed, which can optionally be further substituted. Exemplary compounds according to this embodiment include:

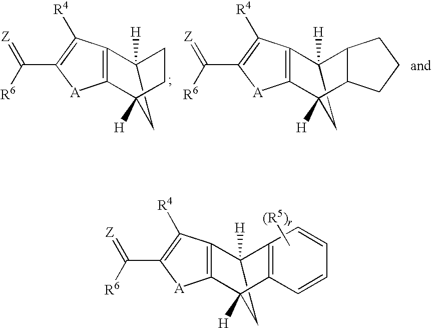

wherein r is a member selected from 0 to 4. Relative stereochemistry is shown.

In yet another exemplary embodiment, at least one of X, Y and Q is C═O or CHOH. Exemplary compounds include:

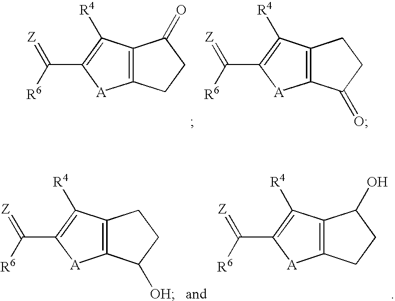

In another example, according to any of the above embodiments, e.g., in Formulae (I) to (V), the compound of the invention is a thiophene or furan analog, in which A is S or O. In one example the compound of the invention has a structure according to the formulae:

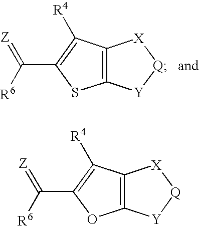

wherein Z, R⁶ and R⁴ are defined as for Formula (I), above. In one embodiment, R⁴ is H. In another embodiment, Z is O. In yet another embodiment, R⁶ is OR⁸ or O⁻X⁺. X, Q and Y are members independently selected from O, S, NR³, CR¹R², C═CR⁴⁰R⁴¹, C═O, C═S and C═NR³. In one example, in the above structures, at least one of X, Q and Y is other than —CH₂—. In another example, in the above structures Z, R⁶ and R⁴, X, Q and Y are defined as for Formula (VI). In yet another example, in the above structures Y is not C═O.

### B. Synthesis

The compounds of the present invention, including compounds of Formula (I) to Formula (V), can be prepared by methods known in the art. One of ordinary skill in the art will know how to modify procedures to obtain the analogs of the present invention. Suitable procedures are described e.g., in Helvetica Chimica Acta 1995, 78: 109-121; Journal of the Chemical Society, Perkin Transactions 1: Organic and Bio-Organic Chemistry (1972-1999) 1989: 1369-1373; Organic Preparations and Procedures International 1997, 29: 471-473; Journal of Medicinal Chemistry 1998, 41: 808-820; Chemische Berichte 1975, 108: 2161-2170; Bulletin de la Societe Chimique de France 1974: 1147-1150; Science of Synthesis 2002, 9: 441-552.; Canadian Journal of Chemistry 1971, 49: 3544-3564; Tetrahedron Letters 1999, 40: 6117-6120; Journal of the American Chemical Society 1968, 90: 6877-6879; Journal of Organic Chemistry 1987, 52: 5395-5400; Journal of the Chemical Society, Perkin Transactions 1: Organic and Bio-Organic Chemistry 1995: 1131-1136; Tetrahedron Letters 1993, 34: 6603-6606; Tetrahedron Letters 1968: 1317-1319; Journal fuer Praktische Chemie (Leipzig) 1972, 314: 353-364; Journal of the Chemical Society, Perkin Transactions 1: Organic and Bio-Organic Chemistry (1972-1999) 1974: 490-501; Energy & Fuels 1990, 4: 668-674; Journal of Organic Chemistry 1992, 57: 4809-4820; Tetrahedron 1993, 49: 4159-4172; Energy & Fuels 1993, 7: 172-178; Journal of the Chemical Society, Perkin Transactions 1: Organic and Bio-Organic Chemistry (1972-1999) 1984: 111-118; Journal of the American Chemical Society 1992, 114: 9859-9869; Journal of Organic Chemistry 1987, 52: 5364-5374; Journal of Organic Chemistry 1987, 52: 3986-3993; Water Science and Technology 1996, 33: 9-15; Liebigs Annalen der Chemie 1980: 564-589; Journal of Heterocyclic Chemistry 1993, 30: 477-482; Khimiya Geterotsiklicheskikh Soedinenii 1972: 342-344; Journal of Organic Chemistry 1983, 48: 4779-4781; Ann. 1935, 517: 152-169; Tetrahedron Letters 1985, 26: 1839-1842; Angew Chem, IntEd Engl 1993, 32: 1051-1052 (See also Angew Chem, 1993, 1105(1057), 1116-1117); Youji Huaxue 1997, 17: 524-528; Tetrahedron Letters 2003, 44: 7253-7256; WO/9940913; WO/9948868; U.S. Pat. No. 4,587,258; WO/8600896; CN/94-107461 (1106386); and DE/84-3431541; each of which is incorporated herein by reference in its entirety. Pure enantiomers of chiral compounds can also be obtained by chiral separation methods known in the art, such as chiral HPLC. In addition, compounds can be prepared using the methods described herein below in Schemes 1 through 18 and Examples 1 through 5 or modified versions thereof.

### Synthesis of Fused Analogs

In an exemplary embodiment, fused pyrrole analogs of the invention are prepared using procedures outlined in Schemes 1 through Scheme 18, below. Esters in these examples can be hydrolyzed using standard ester hydrolysis conditions such as those described in General Procedure 7.

In one example, compounds of the invention are prepared using the procedures outlined in Org. Preparations and Procedures International, 1997, 29: 471-473 and references cited therein. For example, compounds of the invention are synthesized according to a procedure outlined in Scheme 1, below.

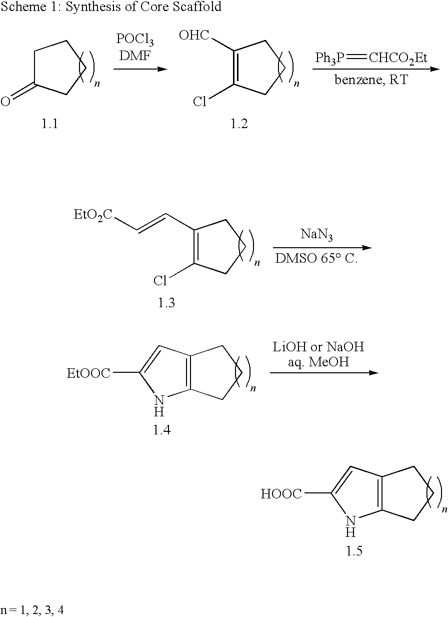

In Scheme 1, chloroformylation of a cyclic ketone such as 1.1, with a reagent such as phosphoryl trichloride in DMF, provides a β-chlorovinyl aldehyde such as 1.2. Olefination of the resulting aldehyde with an olefination reagent such as (carbethoxymethylene)-triphenylphosphorane provides an acrylic acid ester such as 1.3. Cyclization of the acrylic acid ester 1.3 with sodium azide in DMSO provides the cyclized ester 1.4. Hydrolysis of the ester under standard conditions (e.g., aqueous, alcoholic lithium hydroxide or sodium hydroxide) provides the desired acid, such as 1.5.

In another example, compounds of the invention are prepared using the procedures outlined in J.C.S. Perkins Trans. 1, 1989, 8: 1369-1373; J. Org. Chem., 1965, 30: 1126-1129; and WO 99/40913 and references cited within. For example, compounds of the invention are synthesized according to a procedure outlined in Scheme 2, below.

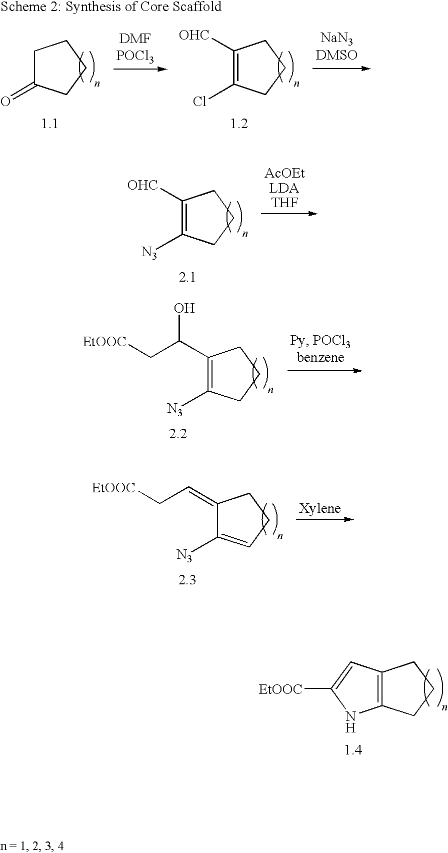

In Scheme 2, chloroformylation of a cyclic ketone such as 1.1 with phosphoryl trichloride in DMF provides a β-chlorovinyl aldehyde, such as 1.2. Treatment of the β-chlorovinyl aldehyde with sodium azide in DMSO provides the corresponding 2-azido cycloalkene 1-carbaldehyde 2.1. Aldol condensation with ethyl acetate provides alcohol 2.2. Dehydration, with phosphoryl trichloride in pyridine provides 2.3, which undergoes thermal cyclization in xylene to provide analog 1.4, which can be hydrolized to the corresponding acid as described herein.

In another example, compounds of the invention are prepared using the procedures outlined in Tetrahedron Lett., 1985, 26: 1839-1842; Tetrahedron Lett., 1968, 11: 1317-1319; and U.S. Pat. No. 5,550,255 as well as references cited therein. For example, compounds of the invention are synthesized according to a procedure outlined in Scheme 3, below.

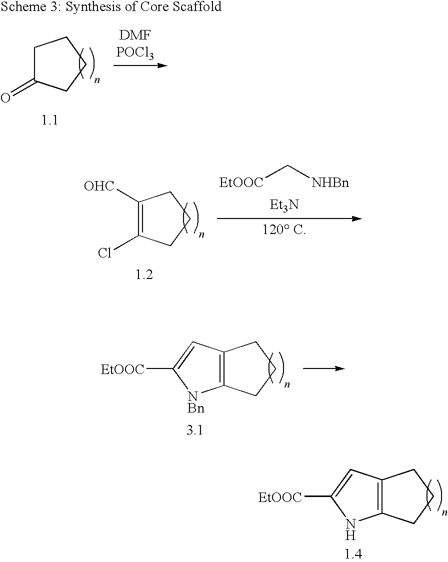

In Scheme 3, chloroformylation of a cyclic ketone such as 1.1 with phosphoryl trichloride in DMF provides a β-chlorovinyl aldehyde, such as 1.2. Condensation with a protected glycine ester, such as N-benzylglycine ethyl ester, followed by cyclization provides a protected pyrrole, such as 3.1. Subsequent deprotection of the pyrrole nitrogen provides analog 1.4, which can be hydrolized to the corresponding acid 1.5 as described herein.

In another example, compounds of the invention are prepared using the procedures outlined in WO 86/00896 to Gold, Neustadt, and Smith. For example, compounds of the invention are synthesized according to a procedure outlined in Scheme 4, below.

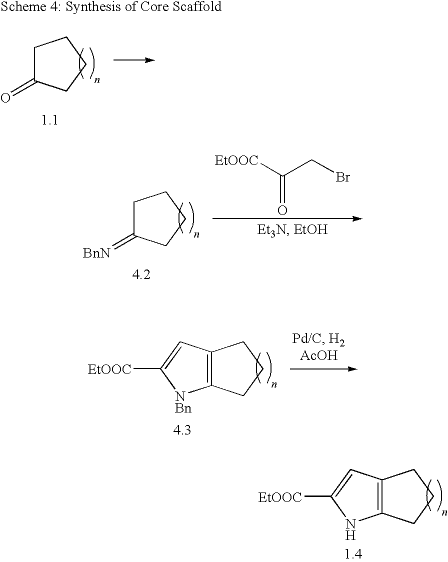

In Scheme 4, condensation of an alkyl amine (e.g., benzylamine) with a cyclic ketone (e.g., 1.1) provides the corresponding cycloalkylimine 4.2. Reaction with a halopyruvate ester (e.g., ethyl bromopyruvate) provides the cyclized product 4.3. The protecting group derived from the alkyl amine can be removed to provide the deprotected pyrrole (e.g., ester 1.4.). Suitable protecting groups for amines, such as aromatic amines, and corresponding methods for deprotection are know to those of skill in the art. For example, as shown in Scheme 4, N-benzyl pyrroles (e.g., 4.3.) can be deprotected using hydrogenation conditions. The ester group of compound 1.4. can be deprotected using hydrolysis conditions described herein.

Exemplary cyclic ketones in Schemes 1 to 4 include:

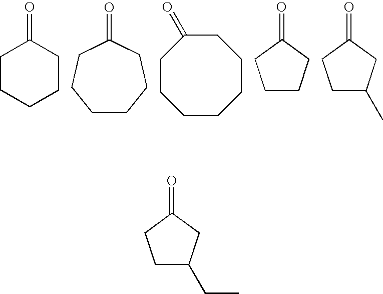

In another example, compounds of the invention are prepared using the procedures outlined in J. Chem. Soc., Perkins Trans. 1, 1984, 1: 111-118, and references cited therein. In one example, compounds of the invention are synthesized according to a procedure outlined in Scheme 5, below.

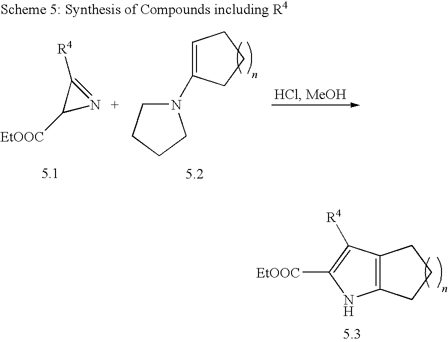

In Scheme 5, reaction of 2H-azirine-3-carboxylic acid esters (e.g., 5.1.) with cycloalkylenylamines (e.g., 5.2.) followed by treatment with acid, such as HCl in methanol, provides analogs 5.3.

In another example, compounds of the invention are prepared using the procedures outlined in J. Heterocyclic Chem. 1993, 30: 477-482; Energy & Fuels 1993, 7: 172-178; and Synthesis 2005: 1569-1571 and references cited therein. In one example, compounds of the invention are synthesized according to a procedure outlined in Scheme 6, below.

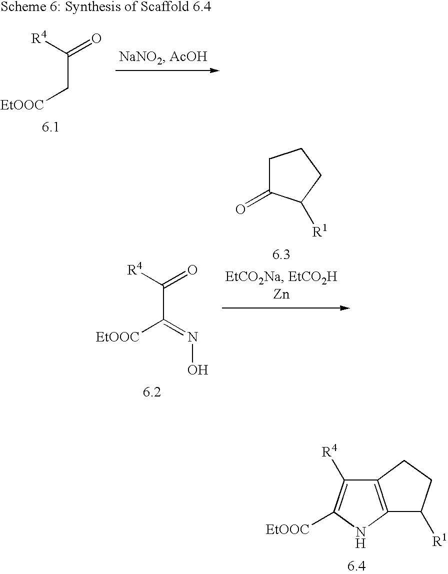

In Scheme 6, reaction of oxime 6.2 (e.g., derived from β-keto ester 6.1), with a cyclic ketone (e.g., 6.3), under Knorr pyrrole formation conditions, provides analog 6.4, which can be converted to the corresponding carboxylic acid analog according to procedures described herein.

### Synthesis of Fused Keto Analogs

In an exemplary embodiment, keto-substituted analogs of the invention are prepared using a procedure outlined in Schemes 7, below.

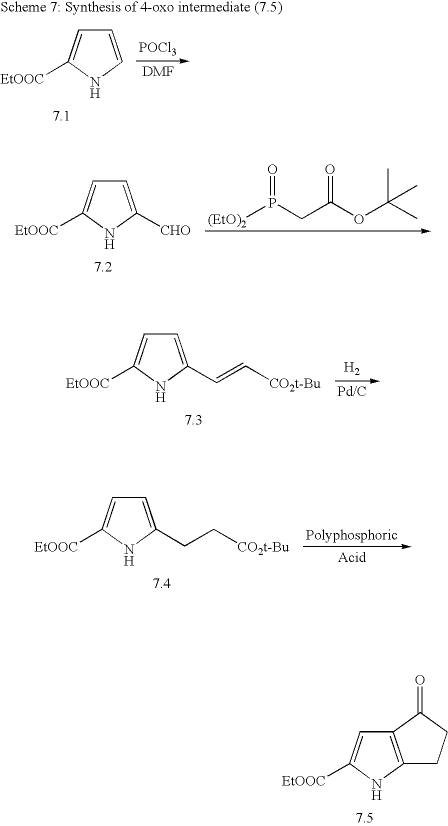

In Scheme 7, Villsmeier formylation of a 1H-pyrrole-2-carboxylic acid ester (e.g., with phosphoryl trichloride) provides aldehyde 7.2. Olefination (e.g., with tert-butyl diethylphosphonoacetate) provides α,β-unsaturated esters, such as 7.3. Hydrogenation to 7.4, followed by cyclization (e.g., using polyphosphoric acid) provides the ketone analog 7.5.

In an exemplary embodiment, 4-keto-substituted analogs of the invention are prepared using a procedure described in J. Org. Chem., 1983, 48: 4779-4781; Synthetic Commun., 2002, 32: 897-902; J. Org. Chem., 1987, 52: 5395-5400 and references cited therein. In one example, analogs of the invention are prepared using procedures outlined in Schemes 8, 9 or 10, below.

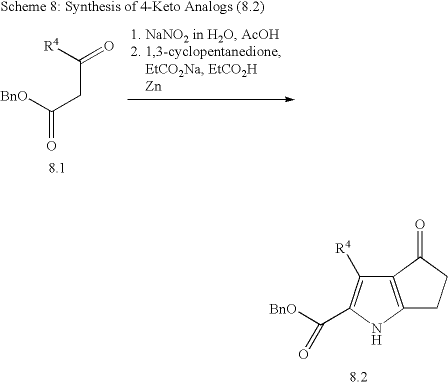

In a method similar to that described in Scheme 6, Scheme 8 describes the reaction of the oxime derived from β-keto ester 8.1 with 1,3-cyclopentanedione, under Knorr pyrrole formation conditions, to provide 4-keto analogs 8.2.

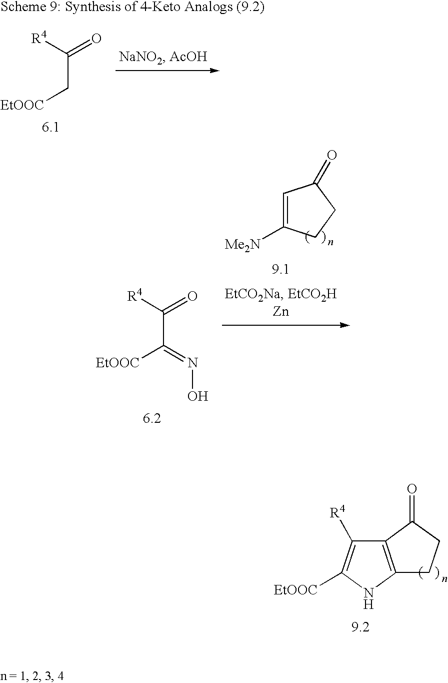

In Scheme 9, reaction of oxime 6.2, derived from β-keto ester 6.1, with the cyclic enamine ketone 9.1, under Knorr pyrrole formation conditions, provides 4-keto analogs 9.2.

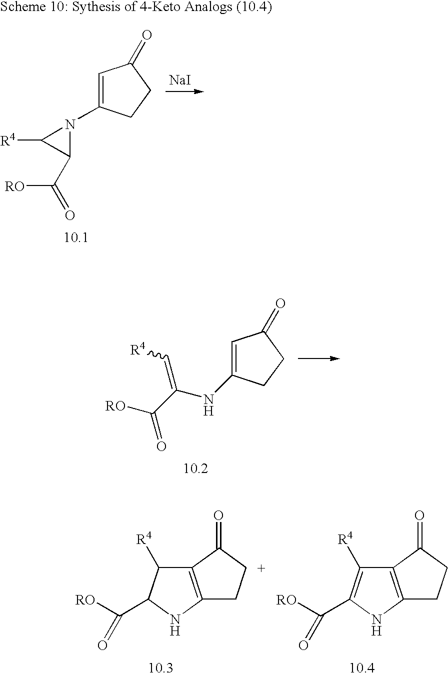

In Scheme 10, N-vinylaziridine 10.1 (e.g., synthesized according to Can. J. Chem. 1982, 60: 2830) is isomerized in the presence of sodium iodide to provide dieneamine 10.2. Photocyclization of dieneamine 10.2 provides a mixture of 10.3 and 4-keto analog 10.4.

5-Keto-analogs of the invention can be prepared using procedures outlined in Tetrahedron 2004, 60: 1505-1511. In one example, compounds of the invention are synthesized according to the procedure outlined in Scheme 11, below.

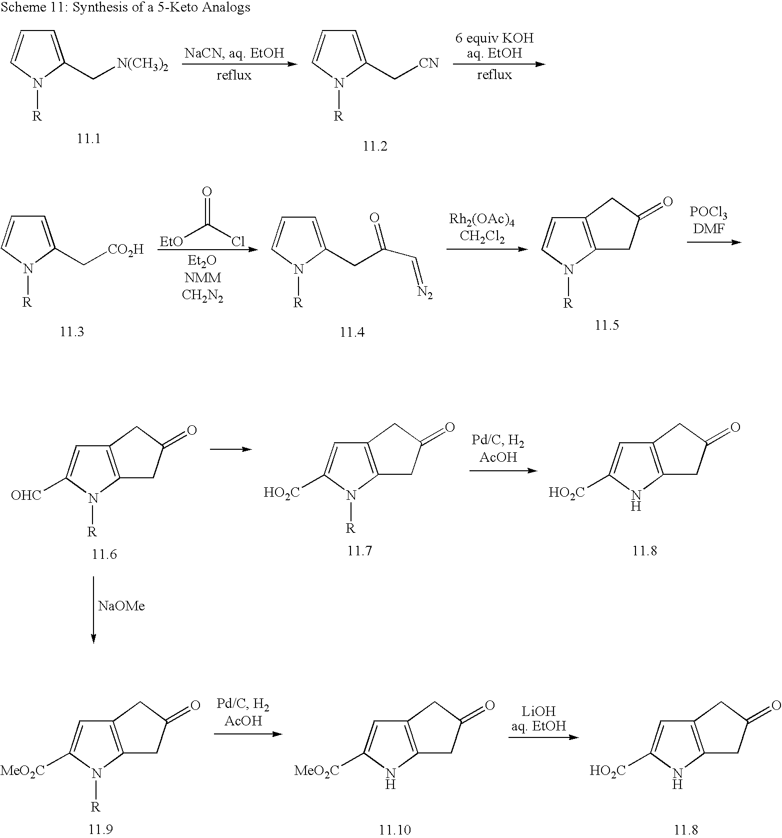

In Scheme 11, nitrile 11.2 can be formed from the corresponding Mannich base 11.1 and sodium cyanide. Alkaline hydrolysis of nitrile 11.2 provides acid 11.3. Pyrroloyl diazoketones 11.4 can be prepared from acid 11.3 by addition of excess ethereal diazomethane to a solution of the mixed ethyl carbonic-carboxylic anhydrides generated in situ with ethyl chloroformate. Treatment of diazo compounds 11.4 with catalytic rhodium (II) acetate provides the keto-substituted fused pyrrole 11.5. Formylation of the pyrrole (see, for example Tetrahedron Lett 2006, 47: 3693-3696, Tetrahedron 2004, 60: 1197-1204, and Bioorg. Med. Chem. Lett. 2004, 14: 187-190) provides the aldehyde 11.6. The aldehyde is converted to the desired acid or ester and the pyrrole nitrogen is deprotected using standard methods, such as those outlined in Scheme 11 to provide 11.8 or 11.10 (see also J. Med. Chem. 2004, 47: 5167-5182; Bull. Chem. Soc. Japan 2002, 75: 2215-2220; J. Org. Chem., 1999, 64: 478-487; Revista de Chimi 2001, 52: 206-209; Organic Preparations and Procedures International 1994, 26: 123-125; J. Heterocyclic Chem. 1986, 23: 769-773; J. Heterocyclic Chem., 1985, 22: 259-263; J. Organometallic Chem. 1981, 212: 1-9; J. Med. Chem. 1980, 23: 462-465; Tetrahedron Lett. 2006, 47: 3521-3523; Heterocycles 2006, 68: 713-719; Tetrahedron Lett. 2006, 47: 1071-1075; J. Am. Chem. Soc. 2006, 128: 6314-6315; Heterocycles 2005, 65: 2693-2703; Org. Lett. 2006, 8: 115-118; Bioorg. Med. Chem. Lett., 2005, 15: 4540-4542. In Scheme 11, benzyl is used as a protecting group. A person of skill in the art will appreciate that other pyrrole protecting groups can also be used.

6-Keto-analogs of the invention can be prepared using procedures outlined in European J. Org. Chem. 2006, 2: 414-422, Tetrahedron 1993, 49: 4159-4172; J. Am. Chem. Soc. 1968, 90: 6877-6879; J. Am. Chem. Soc. 1954, 76: 5641-5646; Ann 1928, 462: 246; Ann 1928, 466: 171; Ann 1932, 492: 154 and references cited therein. In one example, compounds of the invention are synthesized according to the procedure outlined in Schemes 12 and 13, below.

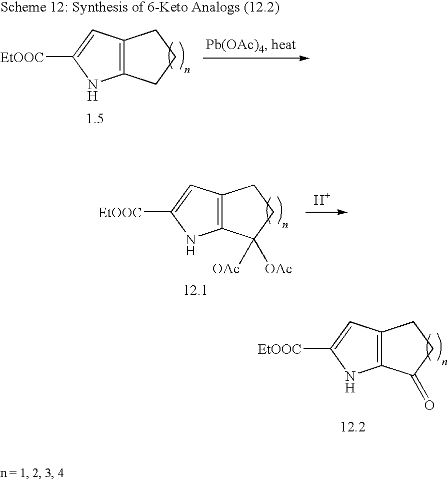

In Scheme 12, oxidation of analog 1.5 with lead tetraacetate, followed by hydrolysis provides the 6-keto analog 12.2.

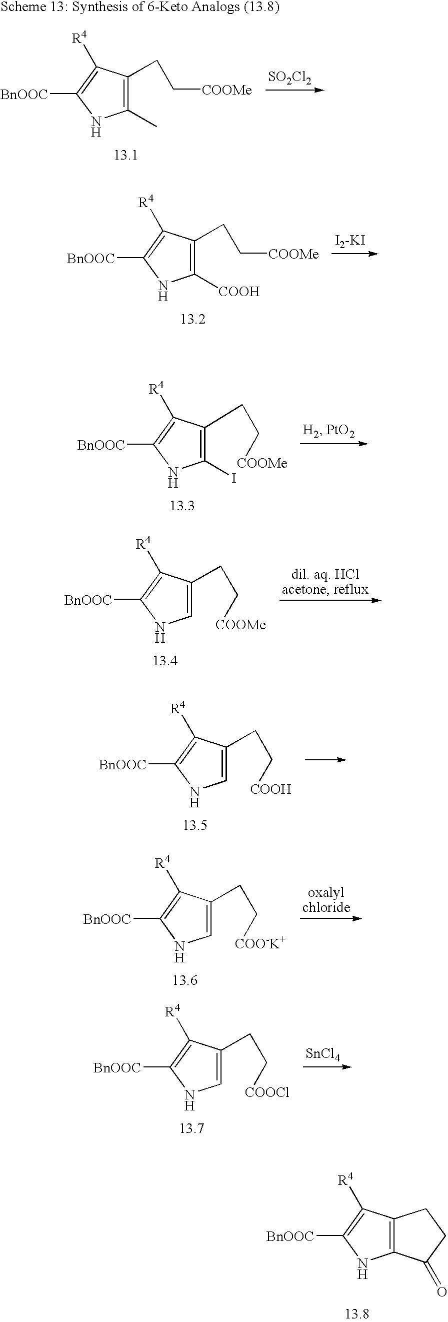

In Scheme 13, the α-methyl group of 13.1 is oxidized to the carboxylic acid of 13.2 (e.g., by treatment with sulfuryl chloride in acetic acid). Iodo-decarboxylation with 12/KI provides 13.3. Hydrogenation removes the iodine and hydrolysis of the methyl ester provides acid 13.5. An alternative route to a related analog, 3(-5-(methoxycarbonyl)-1H-pyrrol-3-yl)propanoic acid, is described in the Examples section. Conversion of 13.5 to the acid chloride, followed by SnCl₄-catalyzed cyclization provides analog 13.8. 13.8 can also be synthesized from acid 13.5 using polyphosphoric acid, as described in the Examples section.

Other conditions for oxidation of core scaffolds such as 1.5, 5.4, and 6.4 to the desired keto-derivatives can also be found in the following references, as well as references cited therein: Heterocycles 1990, 30: 1131-1140; J. Org. Chem. 1990, 55: 3858-3866; and Tetrahedron 1985, 41: 3813-3823.

In Schemes 1-13, R¹ and R⁴ are defined as herein above. In one example, R¹ and R⁴ are members independently selected from H and substituted or unsubstituted alkyl. In another example, R¹ and R⁴ in these Schemes are independently selected from substituted or unsubstituted methyl, ethyl, propyl and butyl. In yet another example, R¹ is methyl. In a further example, R⁴ is methyl.

In addition, esters in these examples can be hydrolyzed using standard ester hydrolysis conditions such as lithium hydroxide or sodium hydroxide in aqueous ethanol or methanol. Exemplary hydrolysis conditions are described herein below, in General Procedure 7.

The above describe keto-substituted analogs of the invention can be used as intermediates in the synthesis of additional analogs through standard functional group manipulations such as protection, deprotection, alkylation, hydrolysis, hydrogenation, and the like. Methods for the conversion (e.g., alkylation) of keto groups are known to those skilled in the art. Exemplary methods are shown in Scheme 14, below. Exemplary keto-intermediates include 7.1, 8.2, 9.2, 10.4, 11.8, 12.2, and 13.8.

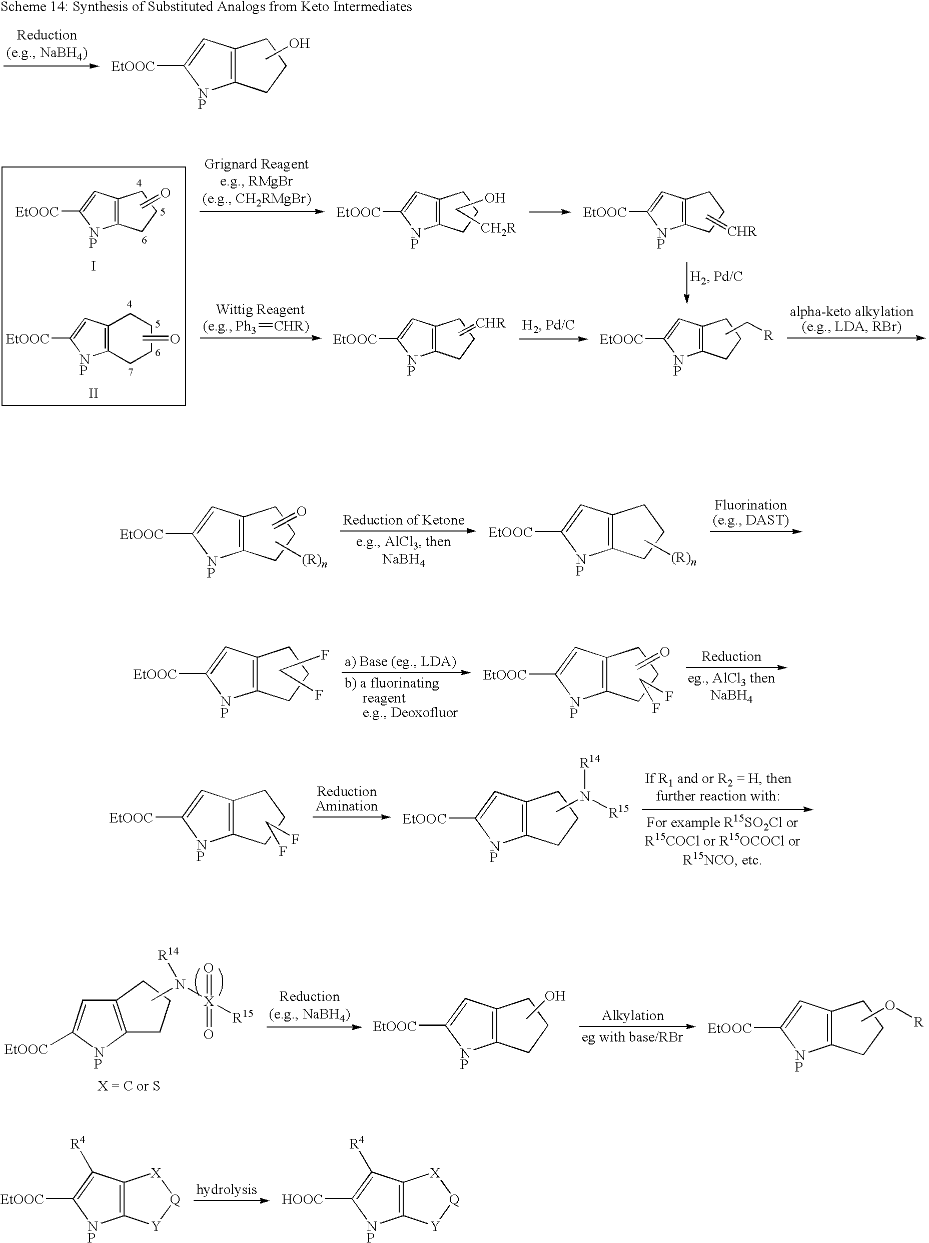

In Scheme 14, R represents H, substituted or unsubstituted alkyl, substituted or unsubstituted heteroalkyl, substituted or unsubstituted aryl, substituted or unsubstituted heteroaryl and substituted or unsubstituted heterocycloalkyl. In one example, R is selected from H, substituted or unsubstituted methyl, ethyl, propyl or butyl and substituted or unsubstituted phenyl. The keto group of compound I is found at position 4, 5 or 6 of the 5-membered ring. The keto group of compound II can be at positions 4, 5, 6 or 7 of the 6-membered ring. In Scheme 14, the group P is a member selected from H and a protecting group. Protecting groups useful for the protection of amines (e.g., aromatic amines) are known to those of skill in the art (see, for example, T W Greene and P G M Wuts, Protective Groups in Organic Synthesis, third edition 1999, John Wiley & Sons). In an exemplary embodiment, the protecting group is selected from Bn and SEM.

In Scheme 14, the ketone can be reduced to the corresponding alcohol, for example, using NaBH₄ (see, e.g., Tetrahedron, 1993, 49: 4159-4172). In another example, the ketone is alkylated using a Grignard reagent. The resulting alcohol can be converted to an alkene, which is optionally reduced to the corresponding alkyl analog (e.g., using palladium on charcoal). In yet another example, the ketone can be alkylated using a Wittig reagent to obtain an alkene, which is optionally reduced to the corresponding alkane. Grignard and Wittig reactions are well known to those of skill in the art. Alternatively, any hydrogen atom in the 5- or 6-membered ring can be replaced with a halogen atom. For example, difluorination can be accomplished using DAST or Deoxofluor. Alternatively, the carbonyl group can be replaced using DAST or Deoxofluor. In another example, reaction of the carbonyl group with a reducing agent in the presence of an amine (reductive amination) can produce a substituted or unsubstituted amine. This amine can be further functionalized with an acid chloride, sulfonyl chloride, isocyanate and the like to produce an amide, sulfonamide, urea or the like. In a further example, the carbonyl can be reduced to an alcohol with a reducing agent such as sodium borohydride and the resulting alcohol can be reacted with a suitable electrophile to produce an ether. Standard hydrolysis conditions, such as those disclosed herein (e.g., lithium hydroxide monohydrate), can be used to convert esters to carboxylic acids.

Fluorinated analogs of the invention can also be prepared using procedures outlined in Tetrahedron 2005, 61: 9338-9348; Heterocycles 1991, 32: 949-963; Tetrahedron 2003, 59: 5215-5223, and references cited therein. In one example, compounds of the invention are synthesized according to the procedures outlined in Schemes 15 and 16, below.

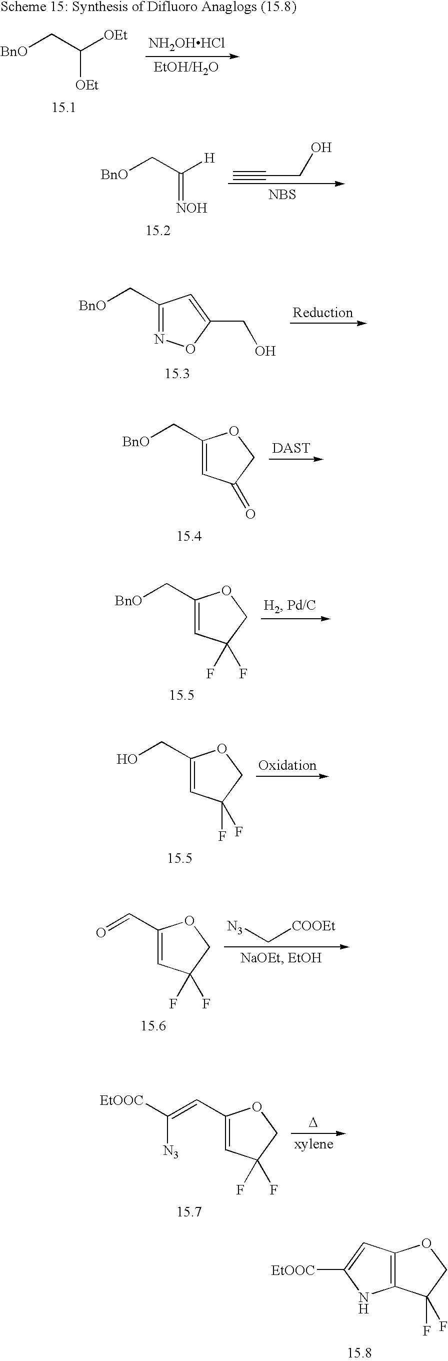

In Scheme 15, deprotection of acetal 15.1 followed by reaction with hydroxylamine provides oxime 15.2. Conversion to the bromooxime with NBS and cycloaddition with the required acetylene provides isoxazole 15.3. Hydrogenation of 15.3 and subsequent cyclization provides 3(2H)-furanone 15.4. Reaction with fluorinating agent DAST provides 15.5. Hydrogenation removes the benzyl protecting group to give 15.5, which is oxidized to aldehyde 15.6, condensed with ethyl 2-azidoacetate to provide 15.7, and cyclized in xylene to obtain analog 15.8. Those skilled in the art would know that other hydroxyl protecting groups can also be used.

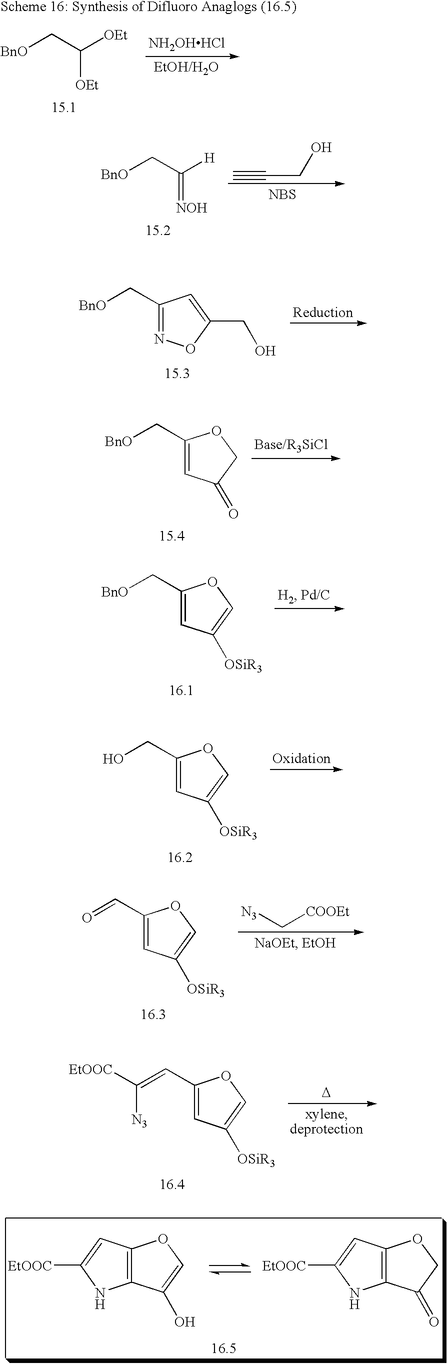

In Scheme 16, 15.4 is converted to furan 16.1 by treatment with base and a silylating reagent. Removal of the protecting group provides alcohol 16.2, which is oxidized to aldehyde 16.3, condensed with ethyl 2-azidoacetate to provide 16.4, cyclized in xylene and deprotected to achieve analog 16.5. Those skilled in the art will appreciate that other hydroxyl protecting groups can be used.

Compounds of the invention including a bicyclic substructure can be synthesised according to described methods (see e.g., Estep, K. G. Syn. Commun. 1995, 25: 507-514; Tetrahedron: Asymmetry 1996, 7: 1269-1272.; Chem. Berichte 1978, 111: 1195-1209.; Chem. Ber. 1975, 108: 1756-1767.; J. Chem. Res. 1997: 102-103.; J. Org. Chem. 2000, 65: 2900-2906; Heterocycles 1989, 28: 1077; and references cited therein). In on example, such compounds are synthesized according to a procedure outlined in Scheme 17, below.

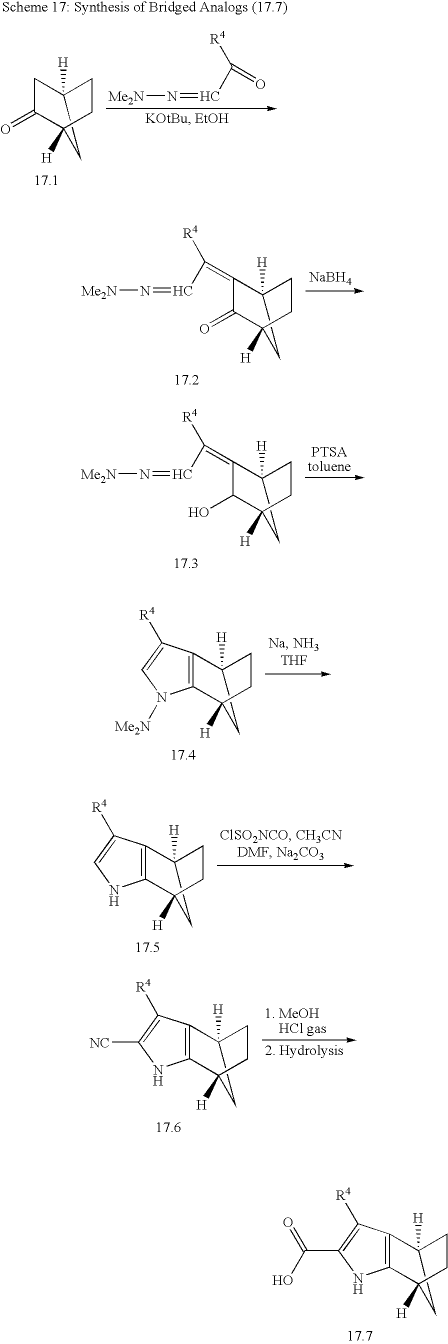

In Scheme 17, Aldol condensation of ketone 17.1 with a keto hydrazone provides 17.2. Reduction of the ketone with sodium borohydride followed by PTSA-catalyzed cyclization provides pyrrole 17.4. Sodium-liquid ammonia reduction provides 17.5. Cyanation of the pyrrole with chlorosulfonyl isocyanate provides 17.6, which can be hydrolyzed to acid 17.7.

Compounds of the invention, including a cyclopentadiene-containing fused rings and fused 5- and 6-membered rings with additionally fused substituted or unsubstituted cyclopentane rings, can be synthesized according to described methods (see e.g., Helvetica Chemica Acta 2004, 87: 1767-1793, and references cited therein) and using other standard functional group manipulations well-known to those skilled in the art. In on example, such compounds are synthesized according to a procedure outlined in Scheme 18, below.

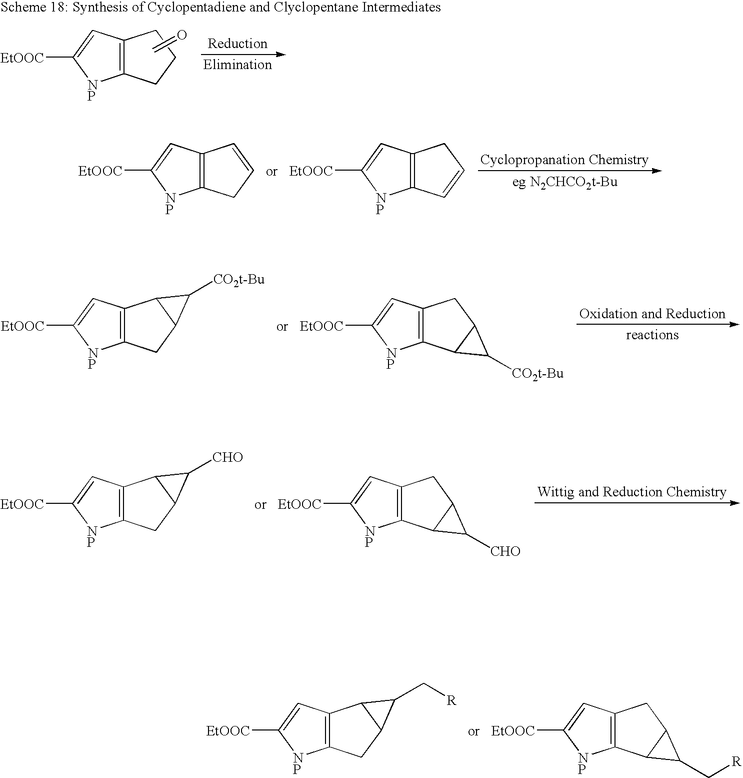

In Scheme 18, R represents H, substituted or unsubstituted alkyl, substituted or unsubstituted heteroalkyl, substituted or unsubstituted aryl, substituted or unsubstituted heteroaryl and substituted or unsubstituted heterocycloalkyl. The keto group is found at position 4, 5 or 6 of the 5-membered ring. In Scheme 18, the group P is a member selected from H and a protecting group. Protecting groups useful for the protection of amines (e.g., aromatic amines) are known to those of skill in the art (see, for example, T W Greene and P G M Wuts, Protective Groups in Organic Synthesis, third edition 1999, John Wiley & Sons). In an exemplary embodiment, the protecting group is selected from Bn and SEM. In Scheme 18, the ketone can be reduced to an alcohol with a reducing agent such as sodium borohydride. The resulting alcohol can then be eliminated to produce an olefin. This olefin is optionally reacted with a diazo compound (e.g., diazoacetate) to produce a cyclopropyl ester. The cyclopropyl ester can be converted to an alcohol by reduction and further to the corresponding aldehyde by oxidation. Functionalization of the aldehyde produces additional cyclopropyl analogs. For example, the aldehyde can be reacted with an appropriate Wittig reagent, and then reduced (e.g., hydrogen gas and a catalyst).

Hydroxy and alkoxy substituted analogs of the invention may be prepared using procedures outlined in Liebigs Ann Chem 1980, 4: 564-589, and references cited within. In one example, compounds of the invention are synthesized according to the procedure outlined in Scheme 19 below.

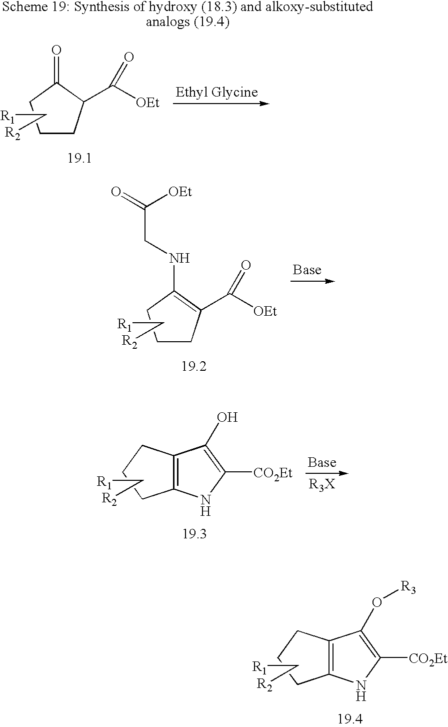

In Scheme 19, R₁, R₂ and R₃ represent H, substituted or unsubstituted alkyl, substituted or unsubstituted heteroalkyl, substituted or unsubstituted aryl, substituted or unsubstituted heteroaryl and substituted or unsubstituted heterocycloalkyl. In one example, R is selected from H, substituted or unsubstituted methyl, ethyl, propyl or butyl and substituted or unsubstituted phenyl. R₁ and R₂ can be found at position 4, 5 or 6 of the 5-membered ring. Additionally, R₁ and R₂ can both occupy position 4, 5 or 6 of the 5-membered ring.

In Scheme 18, the starting keto ester can be reacted with glycine ethyl ester to produce an enamine. This enamine can then be further treated with a base such as sodium ethoxide to form the pyrrole ring. The hydroxyl of this compound can be further elaborated to form ethers through reaction of a base such as sodium hydride and an electrophile such as methyl iodide.

The reagents and reaction conditions, such as those given in Schemes 1 to 18 are exemplary and can be replaced with other suitable reagents and conditions, known to those of skill in the art.

### C. Pharmaceutical Compositions

While it is possible for compounds of the present invention to be administered as the raw chemical, it is preferable to present them as a pharmaceutical composition. According to a further aspect, the present invention provides a pharmaceutical comprising a compound of the invention, e.g., those of Formula (I) to Formula (Vb), or a pharmaceutically acceptable salt, solvate, hydrate or prodrug thereof, together with one or more pharmaceutical carrier and optionally one or more therapeutic ingredient. The carrier(s) must be “acceptable” in the sense of being compatible with the other ingredients of the formulation and not deleterious to the recipient thereof. The term “pharmaceutically acceptable carrier” includes vehicles and diluents.

The formulations include those suitable for oral, parenteral (including subcutaneous, intradermal, intramuscular, intravenous and intraarticular), rectal and topical (including dermal, buccal, sublingual and intraocular) administration, as well as those for administration by inhalation. The most suitable route can depend upon the condition and disorder of the recipient. The formulations can conveniently be presented in unit dosage form and can be prepared by any of the methods well known in the art of pharmacy. All methods include the step of bringing into association a compound or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Oral formulations are well known to those skilled in the art, and general methods for preparing them are found in any standard pharmacy school textbook, for example, Remington: The Science and Practice of Pharmacy, A. R. Gennaro, ed. (1995), the entire disclosure of which is incorporated herein by reference.

Pharmaceutical compositions containing compounds of the invention, e.g., those of Formula (I) to Formula (Vb), can be conveniently presented in unit dosage form and prepared by any of the methods well known in the art of pharmacy. Preferred unit dosage formulations are those containing an effective dose, or an appropriate fraction thereof, of the active ingredient, or a pharmaceutically acceptable salt thereof. The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 1 mg per day to about 7000 mg per day, preferably about 1 mg per day to about 100 mg per day, and more preferably, from about 10 mg per day to about 100 mg per day, and even more preferably from about 20 mg to about 100 mg, 20 mg to about 80 mg or 20 mg to about 60 mg. In some embodiments, the total daily dose can range from about 50 mg to about 500 mg per day, and preferably about 100 mg to about 500 mg per day. It is further recommended that children, patients over 65 years old, and those with impaired renal or hepatic function, initially receive low doses and that the dosage be titrated based on individual physiological responses and/or pharmacokinetics. It can be necessary to use dosages outside these ranges in some cases, as will be apparent to those in the art. Further, it is noted that the clinician or treating physician knows how and when to interrupt, adjust or terminate therapy in conjunction with an individual patient's response.

It should be understood that in addition to the ingredients particularly mentioned above, the formulations of this invention can include other agents conventional in the art having regard to the type of formulation in question, for example those suitable for oral administration can include flavoring agents.

Formulations of the present invention suitable for oral administration can be presented as discrete units such as capsules (e.g., soft-gel capsules), cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

A tablet can be made by compression or molding, optionally using one or more accessory ingredients. Compressed tablets can be prepared by compressing in a suitable machine the active ingredient in a free-flowing form such as a powder or granules, optionally mixed with a binder, lubricant, inert diluent, lubricating, surface active or dispersing agent. Molded tablets can be made by molding in a suitable machine a mixture of the powdered compound moistened with an inert liquid diluent. The tablets can optionally be coated or scored and can be formulated so as to provide sustained, delayed or controlled release of the active ingredient therein. Oral and parenteral sustained release drug delivery systems are well known to those skilled in the art, and general methods of achieving sustained release of orally or parenterally administered drugs are found, for example, in Remington: The Science and Practice of Pharmacy, pages 1660-1675 (1995), the disclosure of which is incorporated herein by reference.

Formulations for parenteral administration include aqueous and non-aqueous sterile injection solutions which can contain anti-oxidants, buffers, bacteriostats and solutes which render the formulation isotonic with the blood of the intended recipient. Formulations for parenteral administration also include aqueous and non-aqueous sterile suspensions, which can include suspending agents and thickening agents. The formulations can be presented in unit-dose of multi-dose containers, for example sealed ampoules and vials, and can be stored in a freeze-dried (lyophilized) condition requiring only the addition of a sterile liquid carrier, for example saline, phosphate-buffered saline (PBS) or the like, immediately prior to use. Extemporaneous injection solutions and suspensions can be prepared from sterile powders, granules and tablets of the kind previously described. Formulations for rectal administration can be presented as a suppository with the usual carriers such as cocoa butter or polyethylene glycol. Formulations for topical administration in the mouth, for example, buccally or sublingually, include lozenges comprising the active ingredient in a flavored basis such as sucrose and acacia or tragacanth, and pastilles comprising the active ingredient in a basis such as gelatin and glycerin or sucrose and acacia.

The pharmaceutically acceptable carrier can take a wide variety of forms, depending on the route desired for administration, for example, oral or parenteral (including intravenous). In preparing the composition for oral dosage form, any of the usual pharmaceutical media can be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents can be used in the case of oral solid preparations such as powders, capsules and caplets, with the solid oral preparation being preferred over the liquid preparations. Preferred solid oral preparations are tablets or capsules, because of their ease of administration. If desired, tablets can be coated by standard aqueous or nonaqueous techniques. Oral and parenteral sustained release dosage forms can also be used.

Exemplary formulations, are well known to those skilled in the art, and general methods for preparing them are found in pharmacy textbooks, for example, Remington, THE SCIENCE AND PRACTICE OF PHARMACY, 21st Ed., Lippincott.

### IV. Methods

### A. Methods of Treatment or Prevention

Subjects for treatment according to methods of the present invention include humans (patients) and other mammals. In one example, the subject is in need of therapy for the stated condition.

In a further aspect the invention provides a method for treating or preventing a disease or condition which is a member selected from a neurological disorder, pain, ataxia and convulsion. The method includes administering to a subject in need thereof a therapeutically effective amount of a compound of the invention (e.g., those of Formula (I) to Formula (Vb)) or a pharmaceutically acceptable salt, solvate, hydrate or prodrug thereof. For example, the compound useful in the above method is a member selected from compounds I-37, disclosed herein.

The invention also provides the use of a compound of the invention in the manufacture of a medicament for the treatment of a disease or condition in a mammal (e.g., a human patient), wherein said disease or condition is a neurological disorder, pain, ataxia or convulsion.

The invention further provides the use of a compound of the invention in the manufacture of a medicament for the enhancement of cognition in a mammal (e.g., a human).

The invention further provides a compound of the invention for use in treating a neurological disorder in a mammal (e.g., human). Exemplary neurological disorders are provided herein.

The invention further provides a compound of the invention for use in treating pain (e.g., neuropathic pain), ataxia or convulsion in a mammal (e.g., a human).

The invention further provides a compound of the invention for use in enhancing cognition in a mammal (e.g., a human).

Compounds of the invention possess unique pharmacological characteristics with respect to inhibition of DAAO and influence the activity of the NMDA receptor in the brain, particularly by controlling the levels of D-serine. Therefore, these compounds are effective in treating conditions and disorders (especially CNS-related disorders), which are modulated by DAAO, D-serine and/or NMDA receptor activity. In one embodiment, compounds of the invention are associated with diminished side effects compared to administration of the current standards of treatment.

Accordingly, the present invention relates to methods for increasing the concentration of D-serine and/or decreasing the concentration of toxic products of D-serine oxidation by DAAO in a mammal. In one embodiment the invention provides a method for treating or preventing a disease or condition, such as those disclosed herein. In one example, the disease or condition is selected from a neurological disorder, pain, ataxia and convulsion. In another embodiment, the invention provides a method of enhancing the cognitive capabilities of a human subject.

In one embodiment, the invention provides a method of enhancing cognition in a mammalian subject (e.g., human). The method includes administering to the subject an effective amount of a compound of the invention (e.g., of Formula (I), Formula (II), Formula (III), Formula (IVa), Formula (IVb), Formula (Va), Formula (Vb) or Formula (VI)), or a pharmaceutically acceptable salt, solvate or prodrug thereof. For example, the compound useful in the above method is a member selected from compounds I-37, disclosed herein. In one example, the subject has been diagnosed with a neurological disorder, such as a neurodegenerative disease disclosed herein (e.g., Alzheimer's disease), with brain injury or spinal cord injury. In another example, the subject benefits from enhanced cognitive capabilities with respect to increased quality of life, performance (e.g., test situations) or coping with stressful situations. For example, the subject is mentally disabled (e.g., due to brain injury). In another example, compounds of the invention are useful in relieving negative symptoms of stress, sleep deprivation (e.g., arising from emergency situations) and disruptions of the circadian rhythm (e.g., jet-lag, night-shifts, time adjustments, such as those to daylight savings time, and the like).

In an exemplary embodiment, the method of the invention includes administering to a mammalian subject (e.g., a human patient) in need thereof a therapeutically effective amount of a compound of the invention, for example a compound of Formula (I), Formula (II), Formula (III), Formula (IVa), Formula (IVb), Formula (Va), Formula (Vb) or Formula (VI), or a pharmaceutically acceptable salt, solvate, hydrate or prodrug thereof. Exemplary prodrugs are esters, for example those in which R⁶ is OR⁸. In this example, R⁸ is selected from substituted or unsubstituted alkyl (e.g., methyl, ethyl, propyl, butyl), substituted or unsubstituted heteroalkyl, substituted or unsubstituted aryl and substituted or unsubstituted heteroaryl.

Compounds of the invention are typically more selective than known DAAO inhibitors, including indole-2-carboxylates, and demonstrate higher selectivity for DAAO inhibition relative to binding at the NMDA receptor's D-serine binding site. The compounds also exhibit an advantageous profile of activity including good bioavailability. Accordingly, they offer advantages over many art-known methods for treating disorders modulated by DAAO, D-serine or NMDA receptor activity. For example, unlike many conventional antipsychotic therapeutics, DAAO inhibitors can produce a desirable reduction in the cognitive symptoms of schizophrenia. Conventional antipsychotics often produce undesirable side effects, including tardive dyskinesia (irreversible involuntary movement disorder), extra pyramidal symptoms, and akathesia, and these can be reduced or eliminated by administering compounds of the invention.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefore, contemporaneously or sequentially with a compound of the present invention. When a compound of the present invention is used contemporaneously with one or more other drugs, a pharmaceutical composition in unit dosage form containing such other drugs and the compound of the present invention is preferred. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s). Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

Compounds of the present invention can also be used in conjunction with therapy involving administration of D-serine or an analog thereof, such as a salt of D-serine, an ester of D-serine, alkylated D-serine, D-cycloserine or a precursor of D-serine

Compounds of the present invention can also be used in conjunction with therapy for neuropathic pain. Agents for this purpose include tricyclic antidepressants, such as imipramine (Tofranil), amitriptyline (Elavil), and nortriptyline (Pamelor, Aventyl); selective serotonin reuptake inhibitors (SSRIs), such as citalopram (Celexa), escitalopram (Lexpro), fluoxetine (Prozac), paroxetine (Paxil) and sertraline (Zoloft); serotinin and norepinephrine reuptake inhibitors (SNRIs), such as Cymbalta (duloxetine); anticonvulsants, such as gabapentin (Neurontin) and pregabalin (Lyrica); opioids such as morphine, oxycodone (OxyContin, Percoset), and fentanyl; and carbamazepine, lidocaine and lamotrigine.

Compounds of the present invention can also be used in conjunction with cognition enhancing agents, e.g., MAO inhibitors, such as selegiline (Eldepryl); cholinesterase inhibitors, such as galantamine (Razadyne), rivastigmine (Exelon), donepezil (Aricept) and Memantine (NMDA antagonist).

Compounds of the present invention can also be used in conjunction with antipsychotics for schizophrenia, which include risperidone (Risperidal), Olanzapine (Zyprexa), Clozapine (Clozaril), Paliperidone (Invega), Quetiapine (Seroquel), Ziprasidone (Geodon), Aripiprazole (Abilify), Asenapine and Lloperidone.

The compounds of the invention can also be used in conjunction with therapy involving administration of antipsychotics (for treating schizophrenia and other psychotic conditions, such as risperidone, olanzapine, clozapine, paliperidone, quetiapine, ziprasidone, aripiprazole, asenapine, loperidone), psychostimulants (for treating attention deficit disorder, depression, or learning disorders), antidepressants, nootropics (for example, piracetam, oxiracetam or aniracetam), acetylcholinesterase inhibitors (for example, galantamine, rivastigmine, the physostigmine related compounds, tacrine or donepezil), GABA analogs (e.g., gabapentin) or GABA receptor modulators, Alzheimer's disease therapeutics (e.g., memantine hydrochloride, and selegiline) and/or analgesics (for treating of persistant or chronic pain, e.g. neuropathic pain). Such methods for conjoint therapies are included within the invention.

In another embodiment, the compounds of the invention can be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies. In another embodiment, the subject compound may be employed in combination with sedatives, hypnotics, anxiolytics, antipsychotics, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chiordiazepoxide, clorethate, chiorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, metha˜ualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, Iricetamide, triclofos, trifluoperazine, trimetozine, trimipramine, uldazepam, venlafaxine, zaleplon, ziprasidone, zola.zepam, zolpidem, and salts thereof, and combinations thereof, and the like, or the subject compound may be administered in conjunction with the use of physical methods such as with light therapy or electrical stimulation. In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO-B inhibitors, antioxidants, A₂ₐ adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form. In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexyl, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the compounds of the invention can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the compounds of the invention can be employed in combination with a compound useful in the treatment of pain, for example carbamazepine, lidocaine, and lamotrigine, an NSAID such as ibuprofen, an antinociceptive agent such as an NR2B antagonist, a COX-2 inhibitor such as ARCOXIA, a Selective Serotonin Reuptake Inhibitor (SSRI) such as citalopram, escitalopram, fluoxetine, paroxetine, and sertraline, a Serotinin and Norepinephrine Reuptake Inhibitor (SNRI) such as Cymbalta, an anticonvulsants such as gabapentin (Neurontin) and pregabalin (Lyrica), an opioids such as morphine, oxycodone, and fentanyl, a tricyclic antidepressants such as imipramine, amitriptyline, and nortriptyline, or a sodium channel blocker.

The compounds of the invention can also be used in conjunction (coadministration) with one or more other therapeutic compound. For example, compounds of the invention can be used in conjunction with therapy involving administration of antipsychotics (e.g., for treating schizophrenia and other psychotic conditions), psychostimulants (e.g., for treating attention deficit disorder, depression, or learning disorders), antidepressants, nootropics (for example, piracetam, oxiracetam or aniracetam), acetylcholinesterase inhibitors (for example, physostigmine related compounds, tacrine or donepezil), GABA analogs (e.g., gabapentin or pregabalin) or GABA receptor modulators, Alzheimer's disease therapeutics (e.g., memantine hydrochloride) and/or analgesics (e.g., for treating persistant or chronic pain, e.g. neuropathic pain). Such methods for conjoint therapies are included within the invention.

In another example, the invention provides a method of inhibiting D-amino acid oxidase (DAAO) enzyme activity, said method comprising contacting said DAAO with a compound of the invention. In one embodiment, the DAAO is located within a cell (e.g., a mammalian cell). In one example according to this embodiment, the cell is located within a mammal. For example, the cell is located within the central (i.e., brain) or peripheral nervous system of a mammal. The invention also provides a composition comprising a compound of the invention and a mammalian cell. The invention further provides a composition comprising a compound of the invention and a DAAO enzyme.

### Conditions and Disorders

In one embodiment, the compounds of the present invention are useful for the treatment of neurological disorders, pain (e.g., neuropathic pain), ataxia and convulsion. Neurological disorders include neurodegenerative diseases (e.g., Alzheimers disease) and neuropsychiatric disorders (e.g., schizophrenia).

Compounds of the invention are useful for the treatment of neurological disorders, pain (e.g., neuropathic pain), ataxia and convulsion, including the treatment of schizoaffective disorder, delusional disorder, brief psychotic disorder, shared psychotic disorder, psychotic disorder due to a general medical condition and substance-induced or drug-induced (phencyclidine, ketamine, and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illnesses associated with psychosis (such as major depression, manic depressive (bipolar) disorder, Alzheimer's disease and post-traumatic stress syndrome), including both the positive and negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age-related cognitive decline; anxiety disorders including acute stress disorder, agoraphobia, generalized anxiety disorder, obsessive-compulsive disorder, panic attack, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general condition, and substance-induced mood-disorders; learning disorders, pervasive development disorder including autistic disorder, attention disorders including attention-deficit hyperactivity disorder (ADHD) and conduct disorder; NMDA-related disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressively supranuclear palsy, multiple system atrophy, corticobasal degeneration, parkinsonism-ALS dementia complex and basal gangli calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias [including tremor (such as rest tremor, postural tremor, and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal cyloclonus), tics (including simple tics, complex tics, and symptomatic tics), and dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

### Neuropsychiatric Disorders

In one example, the compounds of the invention can be used treat neuropsychiatric disorders. Neuropsychiatric disorders include schizophrenia, autism, and attention deficit disorder. Clinicians recognize a distinction among such disorders, and there are many schemes for categorizing them. The Diagnostic and Statistical Manual of Mental Disorders, Revised, Fourth Ed., (DSM-IV-R), published by the American Psychiatric Association, provides a standard diagnostic system upon which persons of skill rely, and is incorporated herein by reference. According to the framework of the DSM-IV, the mental disorders of Axis I include: disorders diagnosed in childhood (such as Attention Deficit Disorder (ADD) and Attention Deficit-Hyperactivity Disorder (ADHD)) and disorders diagnosed in adulthood. The disorders diagnosed in adulthood include (1) schizophrenia and psychotic disorders; (2) cognitive disorders; (3) mood disorders; (4) anxiety related disorders; (5) eating disorders; (6) substance related disorders; (7) personality disorders; and (8) “disorders not yet included” in the scheme.

ADD and ADHD are disorders that are most prevalent in children and are associated with increased motor activity and a decreased attention span. These disorders are commonly treated by administration of psychostimulants such as methylphenidate and dextroamphetamine sulfate.

The compounds (and their mixtures) of the present invention are also effective for treating disruptive behavior disorders, such as attention deficit disorder (ADD) and attention deficit disorder/hyperactivity (ADHD), which is in accordance with its accepted meaning in the art, as provided in the DSM-IV-TR™. These disorders are defined as affecting one's behavior resulting in inappropriate actions in learning and social situations. Although most commonly occurring during childhood, disruptive behavior disorders can also occur in adulthood.

Schizophrenia represents a group of neuropsychiatric disorders characterized by dysfunctions of the thinking process, such as delusions, hallucinations, and extensive withdrawal of the patient's interests from other people. Approximately one percent of the worldwide population is afflicted with schizophrenia, and this disorder is accompanied by high morbidity and mortality rates. So-called negative symptoms of schizophrenia include affect blunting, anergia, alogia and social withdrawal, which can be measured using SANS (Andreasen, 1983, Scales for the Assessment of Negative Symptoms (SANS), Iowa City, Iowa). Positive symptoms of schizophrenia include delusion and hallucination, which can be measured using PANSS (Positive and Negative Syndrome Scale) (Kay et al., 1987, Schizophrenia Bulletin 13:261-276). Cognitive symptoms of schizophrenia include impairment in obtaining, organizing, and using intellectual knowledge which can be measured by the Positive and Negative Syndrome Scale-cognitive subscale (PANSS-cognitive subscale) (Lindenmayer et al, 1994, J. Nerv. Ment. Dis. 182:631-638) or with cognitive tasks such as the Wisconsin Card Sorting Test. Conventional antipsychotic drugs, which act on the dopamine D₂ receptor, can be used to treat the positive symptoms of schizophrenia, such as delusion and hallucination. In general, conventional antipsychotic drugs and atypical antipsychotic drugs, which act on the dopamine D₂ and 5HT₂ serotonin receptor, are limited in their ability to treat cognitive deficits and negative symptoms such as affect blunting (i.e., lack of facial expressions), anergia, and social withdrawal.

Disorders treatable with the compounds of the present invention include, but are not limited to, depression, bipolar disorder, chronic fatigue disorder, seasonal affective disorder, agoraphobia, generalized anxiety disorder, phobic anxiety, obsessive compulsive disorder (OCD), panic disorder, acute stress disorder, social phobia, posttraumatic stress disorder, premenstrual syndrome, menopause, perimenopause and male menopause.

Compounds and compositions of the invention are also effective for treating substance-related disorders and addictive behaviors: Particular substance-related disorders and addictive behaviors are persisting dementia, persisting amnestic disorder, psychotic disorder or anxiety disorder induced by substance abuse; and tolerance of, dependence on or withdrawal from substances of abuse.

Compounds and compositions of the present invention are also effective for treating eating disorders. Eating disorders are defined as a disorder of one's appetite or eating habits or of inappropriate somatotype visualization. Eating disorders include, but are not limited to, anorexia nervosa; bulimia nervosa, obesity and cachexia.

In addition to their beneficial therapeutic effects, compounds of the present invention provide the additional benefit of avoiding one or more of the adverse effects associated with conventional mood disorder treatments. Such side effects include, for example, insomnia, breast pain, weight gain, extrapyramidal symptoms, elevated serum prolactin levels and sexual dysfunction (including decreased libido, ejaculatory dysfunction and anorgasmia).

### Learning, Memory and Cognition

The compounds of the present invention have utility in treating or improving mammalian brain function, especially human cognition. For example, the compounds have utility improving brain function in human disease conditions such as Alzheimer's, schizophrenia, autism, dyslexia, obsessive-compulsive disorder, depression, anxiety, insomnia, sleep deprivation, and in brain injuries.

Generally, compounds of the invention can be used for improving or enhancing learning and memory in subjects with or without cognitive deficits. Patients, who can benefit from such treatment, include those exhibiting symptoms of dementia or learning and memory loss. Individuals with an amnesic disorder are impaired in their ability to learn new information or are unable to recall previously learned information or past events. The memory deficit is most apparent on tasks to require spontaneous recall and can also be evident when the examiner provides stimuli for the person to recall at a later time. The memory disturbance must be sufficiently severe to cause marked impairment in social or occupational functioning and must represent a significant decline from a previous level of functioning. The memory deficit can be age-related or the result of disease or other cause. Dementia is characterized by multiple clinically significant deficits in cognition that represent a significant change from a previous level of functioning, including memory impairment involving inability to learn new material or forgetting of previously learned material. Memory can be formally tested by measuring the ability to register, retain, recall and recognize information. A diagnosis of dementia also requires at least one of the following cognitive disturbances: aphasia, apraxia, agnosia or a disturbance in executive functioning. These deficits in language, motor performance, object recognition and abstract thinking, respectively, must be sufficiently severe in conjunction with the memory deficit to cause impairment in occupational or social functioning and must represent a decline from a previously higher level of functioning.

Compounds of the invention are useful for preventing loss of neuronal function, which is characteristic of neurodegenerative diseases. Therapeutic treatment with a compound of the invention improves and/or enhances memory, learning and cognition. In one embodiment, the compounds of the invention can be used to treat a neurodegenerative disease such as Alzheimer's, Huntington's disease, Parkinson's disease and amyotrophic lateral sclerosis, as well as MLS (cerebellar ataxia), Down syndrome, multi-infarct dementia, status epilecticus, contusive injuries (e.g. spinal cord injury and head injury), viral infection induced neurodegeneration, (e.g. AIDS, encephalopathies), epilepsy, benign forgetfulness, and closed head injury.

Compounds of the invention are useful for treating or preventing loss of memory and/or cognition associated with a neurodegenerative disease. The compounds also ameliorate cognitive dysfunctions associated with aging and improve catatonic schizophrenia.

Alzheimer's disease is manifested as a form of dementia that typically involves mental deterioration, reflected in memory loss, confusion, and disorientation. In the context of the present invention, dementia is defined as a syndrome of progressive decline in multiple domains of cognitive function, eventually leading to an inability to maintain normal social and/or occupational performance. Early symptoms include memory lapses and mild but progressive deterioration of specific cognitive functions, such as language (aphasia), motor skills (apraxia) and perception (agnosia). The earliest manifestation of Alzheimer's disease is often memory impairment, which is required for a diagnosis of dementia in both the National Institute of Neurological and Communicative Disorders and Stroke-Alzheimer's Disease- and the Alzheimer's Disease and Related Disorders Association (NINCDS-ADRDA) criteria (McKhann et al., 1984, Neurology 34:939-944), which are specific for Alzheimer's disease, and the American Psychiatric Association's Diagnostic and Statistical Manual of Mental Disorders, Fourth Edition (DSM-IV) criteria, which are applicable for all forms of dementia. The cognitive function of a patient can also be assessed by the Alzheimer's disease Assessment Scale-cognitive subscale (ADAS-cog; Rosen et al., 1984, Am. J. Psychiatry 141:1356-1364). Alzheimer's disease is typically treated by acetylcholine esterase inhibitors such as tacrine hydrochloride or donepezil. Unfortunately, the few forms of treatment for memory loss and impaired learning available at present are not considered effective enough to make any significant difference to a patient, and there is currently a lack of a standard nootropic drug for use in such treatment.

Other conditions that are manifested as deficits in memory and learning include benign forgetfulness and closed head injury. Benign forgetfulness refers to a mild tendency to be unable to retrieve or recall information that was once registered, learned, and stored in memory (e.g., an inability to remember where one placed one's keys or parked one's car). Benign forgetfulness typically affects individuals after 40 years of age and can be recognized by standard assessment instruments such as the Wechsler Memory Scale. Closed head injury refers to a clinical condition after head injury or trauma. Such a condition, which is characterized by cognitive and memory impairment, can be diagnosed as “amnestic disorder due to a general medical condition” according to DSM-IV.

Compounds and compositions of the invention are also effective for treating cerebral function disorders. The term cerebral function disorder, as used herein, includes cerebral function disorders involving intellectual deficits, and can be exemplified by senile dementia, Alzheimer's type dementia, memory loss, amnesia/amnestic syndrome, epilepsy, disturbances of consciousness, coma, lowering of attention, speech disorders, Parkinson's disease and autism.

In a specific embodiment the present invention provides a method for improving mammalian (e.g., human) brain function related to associative learning, executive function, attention, rehearsal, retrieval, early consolidation, late consolidation, declarative memory, implicit memory, explicit memory, episodic memory, semantic memory, rote learning, informal learning, formal learning, multimedia learning, electronic learning, play, imprinting, social cognition including theory of mind, learning, empathy, cooperativity, altruism, language, non-verbal and verbal communicative skills, telepathy, and sensory integration of environmental cues including temperature, odor, sounds, touch, and taste. The skilled artisan will recognize that there are various methods of measuring improvements in brain function and are practices in behavioral and psychological testing that detect improvements in brain function.

Particular tests of associative learning where the compounds of the present invention have utility are classical or respondant conditioning including forward conditioning, simultaneous conditioning, backward conditioning, temporal conditioning, unpaired conditioning, CS-alone conditioning, discrimination reversal conditioning, interstimulus interval conditioning, latent inhibition conditioning, conditioned inhibition conditioning, blocking, aversion therapy, systematic desensitization, or any other form of conditioning known in the psychological and behavioral literature to those skilled in the art of measuring brain function.

Particular tests of brain function where the compounds of the present invention have utility are measurements of brain function include tests classified as operant conditioning including reinforcement, punishment, and extinction, operant variability, avoidance learning, verbal behavior, four term contingency, operant hoarding, or other tests of modified behaviors.

The compounds also have utility improving brain function in conditions that are not characterized as diseased impairments such as normal aging, low IQ, mental retardation, or any other mental capacity characterized by low brain function. The compounds also have utility in improving brain function during defined tasks performed by humans with normal mental status, such as during extended time periods, in which concentration, attention, problem-solving skills and/or learning is required. For example, compounds of the invention can be used by people operating machinery for extended time periods or people working in emergency or combat situations.

### Pain

The compounds of the invention are useful to treat any kind of acute or chronic pain. In a preferred embodiment, the compounds of the invention are useful to treat chronic pain. In a particularly preferred embodiment, the compounds of the invention are useful to treat neuropathic pain. The term “pain” includes central neuropathic pain, involving damage to the brain or spinal cord, such as can occur following stroke, spinal cord injury, and as a result of multiple sclerosis. It also includes peripheral neuropathic pain, which includes diabetic neuropathy (DN or DPN), post-herpetic neuralgia (PHN), and trigeminal neuralgia (TGN). It also includes dysfunctions of the nervous system such as Complex Regional Pain Syndrome (CRPS), formerly known as Reflex Sympathetic Dystrophy (RSD), and causalgia, and neuropathic pain symptoms such as sensory loss, allodynia, hyperalgesia and hyperpathia. It further includes mixed nociceptive and neuropathic pain types, for example, mechanical spinal pain and radiculopathy or myelopathy, and the treatment of chronic pain conditions such as fibromyalgia, low back pain and neck pain due to spinal nerve root compression, and reflex sympathetic dystrophy.

In one embodiment, the compounds of the present invention are of use in the prevention or treatment of diseases and conditions in which pain and/or inflammation predominates, including chronic and acute pain conditions. In addition to those stated elsewhere, the compounds of the present invention are of use in the treatment and prevention of pain associated with the conditions which include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; chemotherapy-induced neuropathy and “non-painful” neuropathies; pain associated with carcinoma, often referred to as cancer pain; sciatica and ankylosing spondylitis; gout; scar pain; irritable bowel syndrome; bone and joint pain; repetitive motion pain; dental pain; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis and asthma; autoimmune diseases; and immunodeficiency disorders.

Other conditions and disorders include, but are not limited to, autism, childhood learning disorders, depressions, anxieties and sleep disorders. Compounds of the invention are also useful for the treatment of neurotoxic injury that follows cerebral stroke, thromboembolic stroke, hemorrhagic stroke, cerebral ischemia, cerebral vasospasm, hypoglycemia, amnesia, hypoxia (including e.g., sleep/breathing disorders, such as sleep apnea), anoxia, perinatal asphyxia and cardiac arrest.

The term “treating” when used in connection with the foregoing disorders means amelioration, prevention or relief from the symptoms and/or effects associated with these disorders and includes the prophylactic administration of a compound of the invention, a mixture thereof, a solvate (e.g., hydrate), prodrug (e.g., ethyl or methyl esters of the current carboxylic acid inhibitors) or a pharmaceutically acceptable salt of either, to substantially diminish the likelihood or seriousness of the condition.

### B. Models of Disease

Several established animal models of learning and memory are available to examine beneficial, cognitive enhancing effects as well as potential side effects associated with administration of the compounds of the invention. Exemplary methods that can be employed to assess changes in cognition in non-human species are described in the following references, which are incorporated by reference into this application in their entirety: Sarter M, Intern. J. Neuroscience 1987, 32:765-774; Methods and Findings in Experimental and Clinical Pharmacology 1998, 20(3): 249-277; Indian Journal of pharmacology 1997, 29(4): 208-221.

In one example, compounds of the invention are tested using the “Morris Water Maze” (see, e.g., Stewart and Morris, “Behavioral Neuroscience. A Practical Approach. Volume I”, 1993, R. Saghal, Ed., 107-122; Journal of Neuroscience Methods 1984, 11(1): 47-60). The Morris water maze is one of the best-validated models of learning and memory, and it is sensitive to the cognitive enhancing effects of a variety of pharmacological agents. The task performed in the maze is particularly sensitive to manipulations of the hippocampus in the brain, an area of the brain important for spatial learning in animals and memory consolidation in humans. Moreover, improvement in Morris water maze performance is predictive of clinical efficacy of a compound as a cognitive enhancer. For example, treatment with cholinesterase inhibitors or selective muscarinic cholinergic agonists reverse learning deficits in the Morris maze animal model of learning and memory, as well as in clinical populations with dementia. In addition, this animal paradigm accurately models the increasing degree of impairment with advancing age and the increased vulnerability of the memory trace to pre-test delay or interference which is characteristic of amnesiac patients.

In another example, compounds of the invention are tested using “Contextual Fear Conditioning” (see, e.g., Barad, M et al., Proc Natl Acad Sci USA 1998, 95(25): 15020-5 and Bourtchouladze, R et al., Cell, 1994, 79: 59-68). Contextual fear conditioning is a form of associative learning in which animals learn to fear a new environment (or an emotionally neutral conditioned stimulus) because of its temporal association with an aversive unconditioned stimulus (US), such as a foot shock. When exposed to the same context or conditioned stimulus at a later time, conditioned animals show a variety of conditioned fear responses, including freezing behavior. Because robust learning can be triggered with a single training trial, contextual fear conditioning has been used to study temporally distinct processes of short-term and long-term memory. Contextual fear conditioning is believed to be dependent on both the hippocampus and amygdala function.

In another example, compounds of the invention are tested using “Conditioned Fear Extinction” (see, e.g., Walker, D L et al., J Neurosci. 2002, 22(6): 2343-51 and Davis, M et al., Biol. Psychiatry 2006, 60: 369-375). Fear extinction is an example of learning and is a process exhibited in both human and animals, including rodents. Extinction of fear refers to the reduction in the measured level of fear to a cue previously paired with an aversive event when that cue is presented repeatedly in the absence of the aversive event. Extinction of fear is not the erasure of the original fear memory, but instead results from a new form of learning that acts to inhibit or suppress the original fear memory (Bouton, M D and Bolles, R C; J. Exp. Psychol. Anim. Behav. Process. 1979, 5: 368-378; Konorski, J. Inegrative Activity of the Brain: An Interdiscipinary Approach, 1967, Chicago: The University of Chicago Press; Pavlov, I. P. Conditioned Reflexes. 1927, Oxford, United Kingdom: Oxford University Press.). The literature also suggests that glutamate acting at the NMDA receptor is critically involved in learning and memory (Bear, M. F. Proc. Nat. Acad. Sci. 1996, 93: 13453-13459; Castellano, C.; Cestari, V.; Clamei, A. Curr. Drug Targets 2001, 2: 273-283; Morris, R. G.; Davis, S.; Butcher, S. P. Philos. Trans. R Soc. Lond. B Biol. Sci. 1990, 329: 187-204; Newcomer, J. W.; Krystal, J. H. Hippocampus 2001, 11: 529-542). There is also evidence that the NMDA receptor is involved with extinction of fear. For example, NMDA antagonists such as 2-amino-5-phosphopentanoic acid (APV) are known to block fear extinction (Davis, M. et al., Biol. Psychiatry 2006, 60: 369-375; Kehoe, E. J.; Macrae, M.; Hutchinson, C. L. Psychobiol. 1996, 24: 127-135; Lee, H.; Kim, J. J. J. Neurosci. 1998, 18: 8444-8454; Szapiro, G. et al., Hippocampus 2003, 13: 53-58). NMDA agonists (such as the partial agonsist D-cycloserine), are known to facilitate fear extinction (Davis, M et al., Biol. Psychiatry 2006, 60: 369-375; Ledgerwood, L.; Richardson, R.; Cranney, J. Behav. Neurosci. 2003, 117: 341-349; and Walker, D. L. et al., J. Neurosci. 2002, 22: 2343-2351). Additional experimental conditions for fear extinction tests can be found in the references incorporated herein by reference.

In human exposure therapy, a patient is repeatedly exposed for prolonged periods to a feared object or situation in the absence of aversive consequences. As a result, the patient is often able to face their feared cues or situations with less fear and avoidance (extinction retention) due to the learning that took place during exposure therapy (extinction training). It has been shown that agents, such as D-cycloserine, that improve extinction in animals also improve the effectiveness of exposure-based psychotherapy. Examples of exposure based cognitive-behavioral therapy (CBT) improved by agents that improve extinction include exposure to phobic objects as therapy for phobia disorders (see, e.g., Davis, M et al., Biol. Psychiatry 2006, 60: 369-375; Ressler, K. J. et al., Archives Gen. Psychiatry 2004, 61: 1136-1144), exposure to phobic situations as therapy for panic disorders (for social anxiety disorder, see e.g., Hoffmann, S. G. et al., Arch. Gen. Psychiatry 2006, 63: 298-304; Hofmann, S. G.; Pollack, M. H.; Otto, M. W. CNS Drug Reviews 2006, 12: 208-217), recollection of traumatic memories as therapy for post-traumatic stress disorder, exposure to cues associated with drug cravings as therapy for drug addiction, and exposure to cues associated with smoking as therapy for smoking cessation. Because of the cognitive, learning aspects associated with psychotherapy based treatment for disorders such as phobias, anxiety, post-traumatic stress disorder and addiction, compounds of the invention are useful as an adjunct with psychotherapy for the treatment of these conditions. For example, compounds of the invention are useful as an adjunct to shorten the number of therapy sessions required or to improve the therapeutic outcome of therapy.

In another example, compounds of the invention are tested using “Delayed Non-Match to Sample” (see e.g., Bontempi, B. et al., Journal of Pharmacology and Experimental Therapeutics 2001, 299(1): 297-306; Alvarez, P. et al., Proc Natl Acad Sci USA 1994, 7; 91(12), 5637-41); “Delayed Alternation” (also called delayed non-matching to position) (see, e.g., Roux, S. et al., Pharmacol Biochem Behav. 1994, 49(3): 83-88; Ohta, H. et al., Jpn J Pharmacol. 1991, 56(3): 303-9); “Social Discrimination Models” (see, e.g., Engelmann, M. et al., Physiol Behav. 1995, 58(2): 315-21); “Social Recognition Test” (also called delay-induced forgetting) (see e.g., Lemaire, M. et al., Psychopharmacology (Berl). 1994, 115(4):435-40).

In humans, improved learning and memory can be measured by such tests as the Wechsler Memory Scale and the Minimental test. A standard clinical test for determining if a patient has impaired learning and memory is the Minimental Test for Learning and Memory (see e.g., Folstein et al., J. Psychiatric Res. 1975, 12:185), especially for those suffering from head trauma, Korsakoff's disease or stroke. The test result serves as an index of short-term, working memory of the kind that deteriorates rapidly in the early stages of dementing or amnesiac disorders. Ten pairs of unrelated words (e.g., army-table) are read to the subject. Subjects are then asked to recall the second word when given the first word of each pair. The measure of memory impairment is a reduced number of paired-associate words recalled relative to a matched control group. Improvement in learning and memory constitutes either (a) a statistically significant difference between the performance of treated patients as compared to members of a placebo group; or (b) a statistically significant change in performance in the direction of normality on measures pertinent to the disease model.

Animal models or clinical instances of disease exhibit symptoms which are by definition distinguishable from normal controls. Thus, the measure of effective pharmacotherapy will be a significant, but not necessarily complete, reversal of symptoms. Improvement can be facilitated in both animal and human models of memory pathology by clinically effective “cognitive enhancing” drugs which serve to improve performance of a memory task. For example, cognitive enhancers which function as cholinomimetic replacement therapies in patients suffering from dementia and memory loss of the Alzheimer's type significantly improve short-term working memory in such paradigms as the paired-associate task. Another potential application for therapeutic interventions against memory impairment is suggested by age-related deficits in performance which are effectively modeled by the longitudinal study of recent memory in aging mice.

The Wechsler Memory Scale is a widely used pencil-and-paper test of cognitive function and memory capacity. In the normal population, the standardized test yields a mean of 100 and a standard deviation of 15, so that a mild amnesia can be detected with a 10-15 point reduction in the score, a more severe amnesia with a 20-30 point reduction, and so forth. During the clinical interview, a battery of tests, including, but not limited to, the Minimental test, the Wechsler memory scale, or paired-associate learning are applied to diagnose symptomatic memory loss. These tests provide general sensitivity to both general cognitive impairment and specific loss of learning/memory capacity (Squire, 1987). Apart from the specific diagnosis of dementia or amnestic disorders, these clinical instruments also identify age-related cognitive decline which reflects an objective diminution in mental function consequent to the aging process that is within normal limits given the person's age (DSM IV, 1994). As noted above, “improvement” in learning and memory within the context of the present invention occurs when there is a statistically significant difference in the direction of normality in the paired-associate test, for example, between the performance of therapeutic agent treated patients as compared to members of the placebo group or between subsequent tests given to the same patient.

In animals, many established models of schizophrenia are available to examine the beneficial effects of treatment; many of which are described in the following references, as well as references cited therein: Saibo Kogaku 2007, 26(1): 22-27; Cartmell, J. et al., J. Pharm. Exp. Ther. 1999, 291(1): 161-170; Rowley, M; Bristow, L. J.; Hutson, P. H. J. Med. Chem. 2001, 1544(4): 477-501; Geyer, M. A.; Ellenbroek, B; Prog Neuropsychopharmacol Biol Psychiatry 2003, 27(7):1071-1079; Geyer, M. A. et al., Psychopharmacology (Berl). 2001, 156(2-3):117-54; Jentsch, J. D.; Roth, R. H. Neuropsychopharmacology 1999, 20(3):201-25. The tests include “Prepulse Inhibition” (see e.g., Dulawa, S. C.; Geyer, M. A. Chin J Physiol. 1996, 39(3):139-46); “PCP Stereotypy Test” (see e.g., Meltzer et al., (“PCP (Phencyclidine): Historical and Current Perspectives”, ed. E. F. Domino, NPP Books, Ann Arbor, 1981: 207-242); “Amphetamine Stereotypy Test” (see e.g., Simon and Chermat, J. Pharmacol. (Paris) 1972, 3: 235-238); “PCP Hyperactivity” (se e.g., Gleason, S. D.; Shannon, H. E. Psychopharmacology (Berl). 1997, 129(1):79-84); and “MK-801 Hyperactivity” (see e.g., Corbett, R. et al., Psychopharmacology (Berl). 1995, 120(1):67-74), the disclosures of which are each incorporated herein by reference.

The prepulse inhibition test can be used to identify compounds that are effective in treating schizophrenia. The test is based upon the observations that animals or humans that are exposed to a loud sound will display a startle reflex and the observation that animals or humans exposed to a series of lower intensity sounds prior to the higher intensity test sound will no longer display as intense of a startle reflex. This is termed prepulse inhibition. Patients diagnosed with schizophrenia display defects in prepulse inhibition, that is, the lower intensity prepulses no longer inhibit the startle reflex to the intense test sound. Similar defects in prepulse inhibition can be induced in animals via drug treatments (scopolamine, ketamine, PCP or MK-801) or by rearing offspring in isolation. These defects in prepulse inhibition in animals can be partially reversed by drugs known to be efficacious in schizophrenia patients. It is felt that animal prepulse inhibition models have face value for predicting efficacy of compounds in treating schizophrenia patients.

In animals, many established models of pain are available to examine the beneficial effects of treatment; many of which are reviewed in Methods in Pain Research, CRC Press, 2001, Kruger, L. (Editor). Tests of acute pain include the tail flick (see e.g., d'Amour and Smith, J. Pharmacol. Exp. Ther. 1941, 72: 74-79), hot plate (see e.g., Eddy, N. B.; Leimbach, D. J Pharmacol Exp Ther. 1953, 107(3):385-93), and paw withdrawal tests. The phenylbenzoquinone writhing assay is a measure of peritoneovisceral or visceral pain. Persistent pain tests, which use an irritant or foreign chemical agent as the nociceptive stimulus, include the formalin test (see e.g., Wheeler-Aceto, H; Cowan, A Psychopharmacology (Berl). 1991, 104(1):35-44), Freund's adjuvant (see e.g., Basile, A. S. et al., Journal of Pharmacology and Experimental Therapeutics 2007, 321(3): 1208-1225; Ackerman, N. R. et al; Arthritis & Rheumatism 1979, 22(12): 1365-74), capsaicin (see e.g., Barrett, A. C. et al., Journal of Pharmacology and Experimental Therapeutics 2003, 307(1): 237-245), and carrageenin models. These models have an initial, acute phase, followed by a second, inflammatory phase.

Neuropathic pain models are reviewed in Wang and Wang, Advanced Drug Delivery Reviews 2003, and include the “Spinal Nerve Ligation (SNL) model” (also called the “Chung Model”) (see e.g., Kim, S. H.; Chung, J. M. Pain 1992, 50(3):355-63; Chaplan et al., Journal of Neuroscience Methods 1994, 53(1):55-63); “Chronic Constriction Injury (CCI) model” (also called the “Bennett Model”) (see e.g., Bennett, G. J; Xie, Y. K Pain 1988, 33(1):87-107); “Progressive Tactile Hypersensitivity (PTH) model) (see e.g., Decosterd, I. Pain 2002, 100(1): 155-162; Anesth. Analg. 2004, 99: 457-463); “Spared Nerve Injury (SNI) model” (see e.g., Decosterd, I., Pain 2002, 100(1): 155-162; Anesth. Analg. 2004, 99: 457-463); “lumbar nerve ligation model” (see e.g., Ringkamp, M. et al., Pain 1999, 79(2-3): 143-153); and “streptozocin- or chemotherapy induced diabetic neuropathy” (see e.g., Courteix, C.; Eschalier, A.; Lavarenne, J. Pain 1993, 53(1): 81-88; Aubel, B. et al Pain 2004, 110(1-2): 22-32.).

Opioids, such as morphine, display robust efficacy in models of acute pain, such as the tail flick and hot plate tests, as well as in both the initial, acute phase and the second, inflammatory phase of persistent pain tests, such as the formalin test. Opioids also display efficacy in neuropathic pain models, such as the Spinal Nerve Ligation (SNL) model. The general analgesic effects of opiate compounds such as morphine in neuropathic pain models, however, are suggested by the increase in paw withdrawal threshold (PWT) in both the injured and the contralateral (uninjured) paw. Compounds that are useful specifically for the treatment of persistent or chronic pain states (e.g., neuropathic pain), such as gabapentin, tend to display efficacy in models of persistent inflammatory and neuropathic pain, such as the formalin (second phase) and SNL models. Compounds of this type, however, tend to increase PWT in the SNL model in only the injured paw. In addition, these compounds fail to display efficacy in acute tests such as the tail flick test and the hot plate test, and also fail to display efficacy in the initial, acute phase of the formalin test. The lack of effect of compounds in the acute pain tests supports the notion that the antinociceptive action of these compounds is related to specific mechanisms associated with a central sensitized state following injury. As a result, compounds that are efficacious in neuropathic pain model(s), such as the SNL (Chung) model, and the second phase of the formalin test, but are not efficacious in acute pain models, such as hot plate and tail flick, or in the first phase of the formalin test suggest that these compounds are more likely to be effective in persistent and chronic, rather than acute, pain states (see Table 1). In addition, their ability to increase PWT in the SNL model should be specific for the ipsilateral (injured) paw. Relevant references follow, and are included by reference. Singh, L. et al, Psychopharmacology 1996, 127: 1-9. Field, M. J. et al., Br. J. Pharmacol. 1997, 121: 1513-1522. Tyengar, S. et al, J. Pharmacology and Experimental Therapeutics 2004, 311: 576-584. Shimoyama, N. et al Neuroscience Letters 1997, 222: 65-67. Laughlin, T. M. et al., J. Pharmacology and Experimental therapeutics 2002, 302:1168-1175. Hunter, J. C. et al., European J. Pharmacol. 1997, 324: 153-160. Jones, C. K. et al., J. Pharmacology and Experimental therapeutics 2005, 312: 726-732. Malmberg, A. B.; Yaksh, T. L. Anesthesiology 1993, 79: 270-281. Bannon, A. W. et al., Brain Res. 1998, 801: 158-63.

In one embodiment, the compounds of the invention are useful for the treatment of persistent or chronic pain states (e.g., neuropathic pain). As described above, such compounds can be profiled in vivo by evaluating their efficacy in models of both acute and neuropathic pain. Preferred compounds demonstrate efficacy in neuropathic pain models, but not in acute pain models.

TABLE 1
Profile of morphine and gabapentin in a variety of animal models

| Animal Model | Morphine | Gabapentin |
|---|---|---|
| Acute Pain | | |
| Hot plate | + | − |
| Tail flick | + | − |
| Formalin (early phase) | + | − |
| Tissue Injury/Inflammatory Pain | | |
| Formalin (second phase) | + | + |
| Carrageenan | + | + |
| Nerve Injury/Neuropathic Pain | | |
| Spinal Nerve Ligation (SNL; Chung) | + | + |
| Chronic Constriction Injury (CCI; Bennet) | + | + |

There are various animal models with chronic brain dysfunctions thought to reflect the processes underlying human epilepsy and seizures/convulsions, such as those described in Epilepsy Res. 2002, 50(1-2):105-23. Such chronic models include the “kindling model of temporal lobe epilepsy” (TLE); “post-status models of TLE”, in which epilepsy develops after a sustained status epilepticus; and genetic models of different types of epilepsy. Currently, the kindling model and post-status models, such as the pilocarpine or kainate models, are the most widely used models for studies on epileptogenic processes and on drug targets by which epilepsy can be prevented or modified. Furthermore, the seizures in these models can be used for testing of antiepileptic drug effects. A comparison of the pharmacology of chronic models with models of acute (reactive or provoked) seizures in previously healthy (non-epileptic) animals, such as the maximal electroshock seizure test, demonstrates that drug testing in chronic models of epilepsy yields data which are more predictive of clinical efficacy and adverse effects.

The following examples are provided to illustrate selected embodiments of the invention and are not to be construed as limiting its scope.

### EXAMPLES

### General Procedures

Compounds of the invention can be synthesized using the following general procedures.

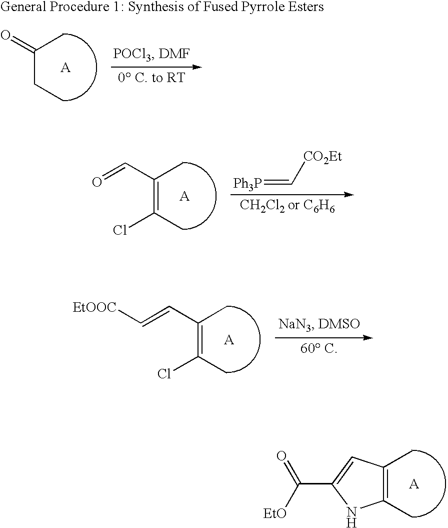

In the above Scheme, ring A represents any substituted or unsubstituted, non-aromatic ring.

### A) Formylation of Ketones

To N,N′-dimethylformamide (DMF) (9.2 mL, 118.9 mmol) at 0° C. was slowly added phosphoryl trichloride (8.9 mL, 95.1 mmol) forming an orange solution that quickly solidified to an orange paste. The ketone (59.4 mmol) was added dropwise over ten min (min). The cooling bath was removed after 30 min. The flask was swirled until liquefaction began to occur. The flask was then reimmersed in an ice bath and allowed to gradually warm to room temperature (rt) overnight, forming a dark solution. The mixture was poured over ice and neutralized with solid NaHCO₃ until no further evolution of CO₂ was observed. The resulting mixture was extracted with ether (5×50 mL). The combined extracts were washed with water and brine, dried (e.g., Na₂SO₄), filtered and passed through a plug of silica before concentration to provide the β-chlorovinyl aldehyde (83%).

### B) Olefination of Aldehydes

To a solution of the above β-chlorovinyl aldehyde (7.66 mmol) in CH₂Cl₂ (10 mL) under nitrogen was added ethoxycarbonylmethylene triphenylphosphorane (2.93 g, 8.42 mmol). The strongly exothermic reaction was refluxed for 6 h, and then stirred at rt for about 12 hours (h). The solvent was removed and the residue adsorbed onto silica and purified by flash chromatography (e.g., 0-30% ethyl acetate (EtOAc)/heptane) to give the ethyl 3-(2-chlorocycloalk-1-enyl)acrylate in 99% yield.

### C) Cyclization

Sodium azide (0.73 g, 11.21 mmol) was added to a solution of the above ethyl 3-(2-chlorocycloalk-1-enyl)acrylate (7.48 mmol) in dimethylsulfoxide (DMSO) (11 mL) and the mixture was heated at 65° C. for about 12 h. After cooling, the mixture was diluted with water and extracted with EtOAc (5×50 mL). The combined organic extracts were washed with water (3×50 mL) and brine, dried (e.g., Na₂SO₄), filtered and concentrated. Purification by flash chromatography (e.g., 0-50% EtOAc/heptane) provided 418 mg (31%) of the fused pyrrole ester.

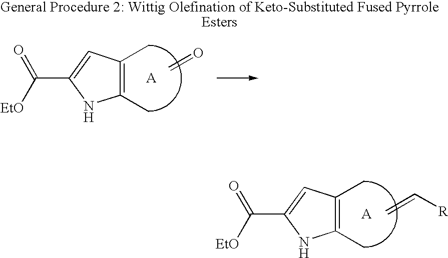

In the above Scheme, ring A represents any substituted or unsubstituted, non-aromatic ring. Exemplary rings for the starting material include cyclopentenones and cyclohexenones.

NaH (145 mg, 3.63 mmol; 60% dispersed in oil), suspended in THF (10 mL) in a 40 mL scintillation vial was reacted with a Wittig reagent (i.e., (4-chlorobenzyl)-triphenylphosphonium chloride) (3.63 mmol) at rt for 2 h. The keto-substituted fused pyrrole ester (i.e., methyl 4-oxo-1,4,5,6-tetrahydrocyclopenta[b]pyrrole-2-carboxylate) (2.79 mmol) was added and the reaction mixture was heated at 65° C. for 48 h. The reaction was concentrated with silica gel and purified by flash chromatography to give the olefin-substituted fused pyrrole ester (i.e., 4-oxo-1,4,5,6-tetrahydrocyclopenta[b]pyrrole-2-carboxylic acid).

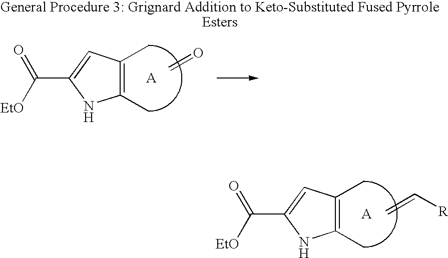

In the above Scheme, ring A represents any substituted or unsubstituted, non-aromatic ring. Exemplary rings for the starting material include cyclopentenones and cyclohexenones.

To a solution of the Grignard reagent (i.e., isobutylMgBr) (4 equiv) in THF (10 mL) at 0° C. was added the ketone (i.e. methyl 4-oxo-1,4,5,6-tetrahydrocyclopenta[b]pyrrole-2-carboxylate) (1.68 mmol) in THF (4 mL). The cooling bath was removed and the reaction mixture was heated to 67° C. for about 12 h. It was then quenched with a saturated solution of NH₄Cl and extracted with EtOAc (3×50 mL). The combined extracts were washed with brine, dried (e.g., Na₂SO₄), filtered and concentrated, optionally onto silica gel. In certain examples, the crude product was purified by flash chromatography (e.g., 0-40% EtOAc/heptane) to afford the olefin-substituted fused pyrrole ester (i.e., methyl 4-(2-methylpropylidene)-1,4,5,6-tetrahydrocyclopenta[b]pyrrole-2-carboxylate). In other examples, the crude reaction mixture was filtered through a silica plug and the dried product was used without further purification.

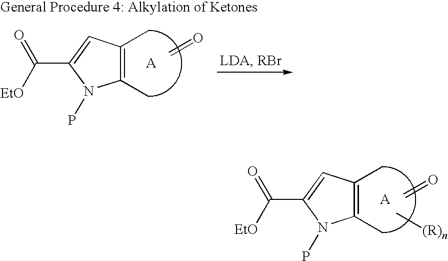

In the above Scheme, ring A represents any substituted or unsubstituted, non-aromatic ring. Exemplary rings include cyclopentenones and cyclohexenones. The R group is a substituent of ring A and is positioned at the alpha-position of the ketone. P is H or a protecting group, such as t-butoxycarbonyl (BOC). When ring A is a 5-membered ring then n is selected from 1 and 2. When A is a 6-membered ring then n is selected from 1, 2 and 3.

To the ketone (i.e., 1-tert-butyl 2-methyl 4-oxo-5,6-dihydrocyclopenta[b]pyrrole-1,2(4H)-dicarboxylate) (1.9 mmol) in THF (20 mL) at −78° C. was added a freshly prepared 0.75 M lithium diisopropylamide (LDA) solution (3.3 mL, 2.47 mmol) and the mixture was stirred at −78° C. for 45 min. The alkyl halide (i.e., iodomethane) (1.9 mmol) was then added. The mixture was allowed to warm to rt and was stirred for about 18 h. The reaction was quenched with a saturated solution of NH₄Cl before extraction with EtOAc (e.g., 3×100 mL). The combined extracts were washed with brine and dried (e.g., over Na₂SO₄). The crude product was optionally purified by column chromatography.

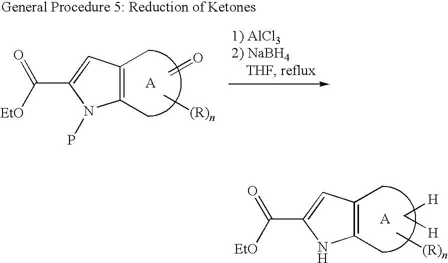

In the above Scheme, ring A represents any substituted or unsubstituted, non-aromatic ring. Exemplary rings include cyclopentenones and cyclohexenones. The R group is positioned adjacent to the newly formed methylene group. When BOC is used as the protecting group (P), a deprotected side product is typically obtained.

To a solution of the above ketone (i.e., 1-tert-butyl 2-methyl 5-methyl-4-oxo-5,6-dihydrocyclopenta[b]pyrrole-1,2(4H)-dicarboxylate) (0.053 g, 0.181 mmol) in THF (5 mL) was added aluminum chloride (0.134 g, 1.0 mmol). The mixture was stirred for 15 min at rt before sodium borohydride (0.070 g, 1.85 mmol) was added. The mixture was heated to reflux for about 4 h. The reaction was allowed to cool to rt and was stirred for about 8 h to about 14 h. The reaction mixture was quenched with saturated aqueous NH₄Cl solution and was extracted with EtOAc (3×25 mL). The combined extracts were washed with brine and dried over Na₂SO₄. The crude product was purified by column chromatography (i.e., 0-30% EtOAc/heptane) to afford the desired product (i.e. methyl 5-methyl-1,4,5,6-tetrahydrocyclopenta[b]pyrrole-2-carboxylate).

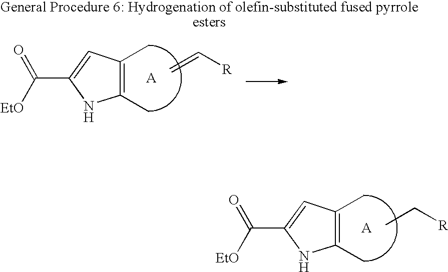

In the above Scheme, ring A represents any substituted or unsubstituted, non-aromatic ring. Exemplary rings include substituted cyclopentenes and cyclohexenes.

To a solution of the olefin-substituted fused pyrrole ester (i.e., 4-(4-chloro-benzylidene)-1,4,5,6-tetrahydro-cyclopenta[b]pyrrole-2-carboxylic acid methyl ester) (0.504 mmol) in EtOAc/MeOH (1:1, 5 mL) or ethanol was added 10% Pd/C under nitrogen. The system was evacuated and refilled with hydrogen three times before allowing the reaction to continue at rt. After the reaction was complete (typically 2.5 h to 3 h), the catalyst was filtered through Celite and the filtrate concentrated. The crude product can be purified by silica gel column chromatography.

When the olefin-containing substituent contains a halogen, (i.e., the substrate 4-(4-chloro-benzylidene)-1,4,5,6-tetrahydrocyclopenta[b]pyrrole-2-carboxylic acid methyl ester) a dehalogenation product (i.e. methyl 4-benzyl-1,4,5,6-tetrahydrocyclopenta[b]pyrrole-2-carboxylate), can also be formed. In those examples it can be necessary to purify the crude product using reverse phase chromatography to separate halogenated analogs from dehalogenated analogs. For example, purification by reverse-phase chromatography (i.e., (75:25 MeOH:water for 5 min)) provided the desired alkyl-substituted fused pyrrole ester (i.e. methyl 4-(4-chlorobenzyl)-1,4,5,6-tetrahydrocyclopenta[b]pyrrole-2-carboxylate).

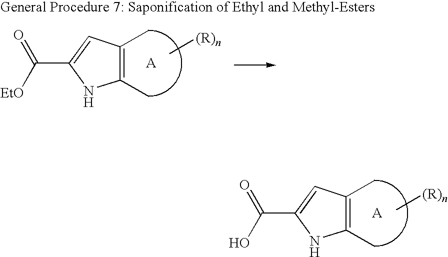

In the above Scheme, ring A represents any substituted or unsubstituted, non-aromatic ring. Exemplary rings include cyclopentenes and cyclohexenes.

To a solution or suspension of the ester (e.g., 0.33 g, 1.2 mmol) in MeOH or EtOH (e.g., 16.5 mL) was added an aqueous base, such as 10 M NaOH (e.g., 0.6 mL, 6 mmol), 5M KOH (e.g., 1.2 mL, 6 mmol) or 1 M LiOH (e.g., 6 mL). The solution was heated to a temperature between about 80° C. and refluxed for a time period between about 30 min and about 20 h (e.g., 5 h). The reaction mixture was cooled to rt and was then acidified. In one example, the mixture was poured into water (e.g., 200 mL) and the pH of the resulting mixture was adjusted to about pH 1-2 with HCl. In another example, excess solvent was removed in vacuo and the residue was dissolved in 5% citric acid (e.g., 15 mL). In yet another example, the solvent was removed in vacuo and the residue was dissolved in a saturated solution of NH₄Cl (e.g., 15 mL). The acidified solution was then extracted (e.g., 3×100 mL EtOAc) and the combined organic layers were washed (e.g., with brine), dried (e.g., over Na₂SO₄), filtered and concentrated in vacuo to give the carboxylic acid.

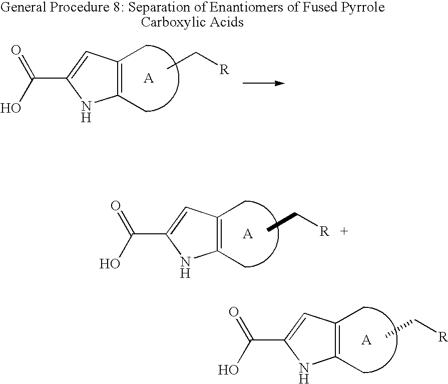

In the above Scheme, ring A represents any substituted or unsubstituted, non-aromatic ring. Exemplary rings include cyclopentenes and cyclohexenes.

Enantiomers of racemic fused pyrrole carboxylic acids were separated using chiral chromatography. An exemplary method uses an isocratic SFC method (40 to 50% methanol in CO₂ with 0.05% diethylamine) on a Chiralpak AD-H column (Chiral Technologies) in a 3.0×25 cm format with a mobile phase flow rate ranging from 70 to 72 g/minute. Alternatively, enantiomers can be separated by chiral chromatography or other art-recognized methods at the ester stage.

### Example 1

### Synthesis of Pyrrole Analogs with Unsubstituted Fused Cyclopentanes

### 1.1.) Synthesis of Ethyl 1,4,5,6-tetrahydrocyclopenta[b]pyrrole-2-carboxylate

The title compound was synthesized from cyclopentanone according to General Procedures 1A, 1B and 1C. In the last step 1.5 g (7.48 mmol) of (E)-ethyl 3-(2-chlorocyclopent-1-enyl)acrylate was cyclized. The crude product was purified by flash chromatography (0-50% EtOAc/heptane) to provide 418 mg (31%, final step) of ethyl 1,4,5,6-tetrahydrocyclopenta[b]pyrrole-2-carboxylate. ¹H NMR (400 MHz, CDCl₃) δ ppm 1.34 (t, J=7.13 Hz, 3H), 2.38-2.48 (m, 2H), 2.59-2.65 (m, 2H), 2.69-2.75 (m, 2H), 4.30 (q, J=7.13 Hz, 2H), 6.67 (d, J=1.37 Hz, 1H), 8.78 (br s, 1H); LCMS-MS (ESI+) 179.9 (M+H).

### 1.2.) Synthesis of 1,4,5,6-tetrahydro-cyclopenta[b]pyrrole-2-carboxylic acid (1)

The title compound was synthesized from the above ethyl-1,4,5,6-tetrahydrocyclopenta[b]pyrrole-2-carboxylate (100 mg, 0.56 mmol) using lithium hydroxide monohydrate (94 mg, 2.23 mmol) as the base according to General Procedure 7. The crude product was purified by flash chromatography (0-100% EtOAc/heptane) to give 61 mg (73%) of 1,4,5,6-tetrahydro-cyclopenta[b]pyrrole-2-carboxylic acid (1). ¹H NMR (400 MHz, CD₃OD) δ ppm 2.35-2.44 (m, 2H), 2.54-2.61 (m, 2H), 2.64-2.72 (m, 2H), 6.59 (s, 1H); LCMS-MS (ESI+) 151.9 (M+H); HPLC (UV=100%), (ELSD=100%).

### Example 2

### Synthesis of Pyrrole Analogs with 4-substituted Fused Cyclopentanes

### 2.1.) Synthesis of Methyl 4-oxo-1,4,5,6-tetrahydrocyclopenta[b]pyrrole-2-carboxylate

### 2.1.a) Synthesis of 5-formyl-1H-pyrrole-2-carboxylic acid methyl ester

To 1,2-dichloroethane (40 mL) was added DMF (13.6 mL, 176 mmol). The mixture was cooled to 0° C. and phosphorus oxychloride (16.4 mL, 176 mmol) was added dropwise over 5 min. The resulting solution was stirred for 15 min. To the solution at 0° C. was added dropwise methyl 1H-pyrrole-2-carboxylate (20 g, 160 mmol) in dichloroethane (80 mL) (about 1 h). The cooling bath was removed, and the reaction mixture was heated to reflux for about 1 h and was then cooled to rt. EtOAc (250 mL) in ice water (400 mL) was added and the organic layer was separated. The aqueous layer was neutralized with NaHCO₃ solution, and then extracted with EtOAc (4×100 mL). The organic layer and the combined organic extracts were washed with dilute NaHCO₃ solution, brine, dried (Na₂SO₄) and filtered. Silica gel was added, the solvent removed and the silica gel-imbedded material was purified by flash chromatography (0-50% EtOAc/Heptane) to afford a major product, methyl 5-formyl-1H-pyrrole-2-carboxylate (16.3 g) and a minor product, methyl 4-formyl-1H-pyrrole-2-carboxylate (6.94 g). Combined yield: 23.3 g (95%).

Methyl 5-formyl-1H-pyrrole-2-carboxylate: ¹H NMR (400 MHz, CDCl₃) δ ppm: 3.93 (s, 3H), 6.95 (d, J=2.39 Hz, 2H), 9.68 (s, 1H), 9.82 (br s, 1H); LCMS-MS (ESI+) 153.9 (M+H).

Methyl 4-formyl-1H-pyrrole-2-carboxylate: ¹H NMR (400 MHz, CHLOROFORM-d) δ ppm: 3.91 (s, 3H), 7.32 (dd, J=2.29, 1.61 Hz, 1H), 7.57 (dd, J=3.32, 1.51 Hz, 1H), 9.55 (br s, 1H), 9.86 (s, 1H); LCMS-MS (ESI+) 153.8 (M+H).

### 2.1.b) Synthesis of (Z)-methyl 5-(3-tert-butoxy-3-oxoprop-1-enyl)-1H-pyrrole-2-carboxylate and (E)-methyl 5-(3-tert-butoxy-3-oxoprop-1-enyl)-1H-pyrrole-2-carboxylate

To a suspension of NaH (5.74 g, 143.5 mmol; 60% in oil) in THF (200 mL) at 0° C. was added (tert-butoxycarbonylmethyl)triphenylphosphonium bromide (66 g, 143.5 mmol) as a solid in three portions. Cooling was removed and the mixture was stirred at rt for 30 min. It was then cooled to 0° C. and methyl 5-formyl-1H-pyrrole-2-carboxylate (16.9 g, 110.4 mmol) in THF (60 mL) was added dropwise over 40 min. The reaction mixture was allowed to warm to rt and was stirred overnight. Silica was added and the solvent was removed. The crude product was purified by flash chromatography (0-20% EtOAc/Heptane) to afford two isomeric compounds:

(Z)-methyl 5-(3-tert-butoxy-3-oxoprop-1-enyl)-1H-pyrrole-2-carboxylate (6.9 g, 25.1%) as a white solid; ¹H NMR (400 MHz, CHLOROFORM-d) δ ppm 1.55 (s, 9H), 3.91 (s, 3H), 5.67 (d, J=12.64 Hz, 1H), 6.43 (dd, J=3.83, 2.32 Hz, 1H), 6.67 (d, J=12.64 Hz, 1H), 6.88 (dd, J=3.81, 2.44 Hz, 1H), 12.80 (br s, 1H); LCMS-MS (ESI+) 195.7 (M-56).

(E)-methyl 5-(3-tert-butoxy-3-oxoprop-1-enyl)-1H-pyrrole-2-carboxylate (20.3 g, 72.9%) as a white solid; ¹H NMR (400 MHz, CHLOROFORM-d) δ ppm 1.53 (s, 9H), 3.90 (s, 3H), 6.19 (d, J=16.01 Hz, 1H), 6.51 (dd, J=3.86, 2.68 Hz, 1H), 6.90 (dd, J=3.88, 2.37 Hz, 1H), 7.41 (d, J=16.01 Hz, 1H), 9.42 (br s, 1H); LCMS-MS (ESI+) 195.8 (M-56).

### 2.1.c) Synthesis of methyl 5-(3-tert-butoxy-3-oxopropyl)-1H-pyrrole-2-carboxylate

To a solution of (Z)-methyl 5-(3-tert-butoxy-3-oxoprop-1-enyl)-1H-pyrrole-2-carboxylate or (E)-methyl 5-(3-tert-butoxy-3-oxoprop-1-enyl)-1H-pyrrole-2-carboxylate (13.6 g, 54.1 mmol, 2 equal batches) in EtOAc (200 mL) under nitrogen was added 10% Pd/C. The flask was flushed with hydrogen three times before allowing the reaction to stir under hydrogen for about 18 h. The catalyst was filtered off using Celite and the filtrate was concentrated to give 27.4 g methyl 5-(3-tert-butoxy-3-oxopropyl)-1H-pyrrole-2-carboxylate as a white solid (100%) for each stereoisomer. ¹H NMR (400 MHz, CHLOROFORM-d) δ ppm 1.46 (s, 9H), 2.54-2.59 (m, 2H), 2.90 (t, J=6.83 Hz, 2H), 3.83 (s, 3H), 5.97 (dd, J=3.49, 2.86 Hz, 1H), 6.81 (dd, J=3.61, 2.59 Hz, 1H), 9.30 (br s, 1H); LCMS-MS (ESI+) 197.86 (M-isobutylene).

### 2.1.d) Synthesis of 3-(5-(methoxycarbonyl)-1H-pyrrol-2-yl)propanoic acid

Methyl 5-(3-tert-butoxy-3-oxopropyl)-1H-pyrrole-2-carboxylate (14.8 g, 58.4 mmol) was treated with 4 N HCl (100 mL) at rt for about 12 h. The solvent was removed and the white solid product was dried to give 12.8 g (94%) of 3-(5-(methoxycarbonyl)-1H-pyrrol-2-yl)propanoic acid. ¹H NMR (400 MHz, CHLOROFORM-d) δ ppm 2.73 (t, J=6.81 Hz, 2H), 2.98 (t, J=6.79 Hz, 2H), 3.83 (s, 3H), 6.01 (dd, J=3.59, 2.61 Hz, 1H), 6.83 (dd, J=3.61, 2.63 Hz, 1H), 9.70 (br s, 1H); LCMS-MS (ESI+) 198.2 (M+H).

### 2.1.e) Synthesis of ethyl 4-oxo-1,4,5,6-tetrahydrocyclopenta[b]pyrrole-2-carboxylate

A suspension of polyphosphoric acid (115%, 109 g) and powdered 3-(5-(methoxycarbonyl)-1H-pyrrol-2-yl)propanoic acid (12.1 g, 51.8 mmol) in 1,2-dichloroethane (40 mL) was heated for 1 h at 100° C. with occasional mixing with a large spatula. Water (100 mL) was added and the mixture was carefully poured into a large Erlenmeyer flask containing solid sodium bicarbonate and ice. The reaction was neutralized (pH 7) and then extracted with EtOAc (5×150 mL). The combined organic extracts were washed with water, NaHCO₃, brine, dried (Na₂SO₄), filtered and concentrated. The crude product was purified by flash chromatography (0-80% EtOAc/Heptane) to afford 8.0 g of methyl 4-oxo-1,4,5,6-tetrahydrocyclopenta[b]pyrrole-2-carboxylate (86%). ¹H NMR (400 MHz, CHLOROFORM-d) δ ppm 2.93-2.98 (m, 2H), 2.99-3.04 (m, 2H), 3.90 (s, 3H), 6.98 (d, J=1.71 Hz, 1H), 9.42 (br s, 1H); LCMS-MS (ESI+) 180.0 (M+H).

### 2.2. Synthesis of methyl 3-tert-butyl-4-oxo-1,4,5,6-tetrahydrocyclopenta[b]pyrrole-2-carboxylate and methyl 4-oxo-1,4,5,6-tetrahydrocyclopenta[b]pyrrole-2-carboxylate

Methyl 5-(3-tert-butoxy-3-oxopropyl)-1H-pyrrole-2-carboxylate (5.1 g, 20.1 mmol) in 5 mL of 1,2-dichloroethane (DCE) was added to polyphosphoric acid (115% assay, 8.5 g) in DCE (15 mL), and the reaction was heated to 100° C. for 2 h. It was then cooled, water (50 mL) was added, and the mixture was extracted with ethyl acetate (4×50 mL). The combined organic extracts were washed with water, dilute NaHCO₃ and brine, dried (Na₂SO₄), filtered and concentrated. Purification by flash chromatography (0-70% EtOAc/heptane) afforded methyl 3-tert-butyl-4-oxo-1,4,5,6-tetrahydrocyclopenta[b]pyrrole-2-carboxylate (1.1 g, 30%). ¹H NMR (400 MHz, CDCl₃) δ ppm 1.50 (s, 3H), 2.86-2.90 (m, 2H), 2.91-2.96 (m, 2H), 3.88 (s, 3H), 9.10 (br s, 1H); LCMS-MS (ESI+) 235.8 (M+H), and methyl 4-oxo-1,4,5,6-tetrahydrocyclopenta[b]pyrrole-2-carboxylate (2.0 g, 42%). ¹H NMR (400 MHz, CCCl₃-d) δ ppm 2.93-2.98 (m, 2H), 2.99-3.04 (m, 2H), 3.90 (s, 3H), 6.98 (d, J=1.71 Hz, 1H), 9.42 (br s, 1H); LCMS-MS (ESI+) 180.0 (M+H). 2.3.

### 2.3. Synthesis of methyl 4-methyl-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (400 mg, 2.23 mmol) and methyl-MgBr (6.38 mL, 8.93 mmol; 1.4 M in toluene/THF: 75:25) according to General Procedure 3 to give methyl 4-methylene-1,4,5,6-tetrahydrocyclopenta[b]pyrrole-2-carboxylate, followed by General Procedure 6 and was purified by column chromatography (0-40% EtOAc/heptane) to afford 19 mg of methyl 4-methyl-1,4,5,6-tetrahydrocyclopenta[b]pyrrole-2-carboxylate as a white solid (5% over two steps). ¹H NMR (400 MHz, METHANOL-d₄) δ ppm 1.17 (d, J=6.78 Hz, 3H), 1.85-1.96 (m, 1H), 2.56-2.76 (m, 3H), 2.95-3.05 (m, 1H), 3.77 (s, 3H), 6.58 (s, 1H); LCMS-MS (ESI+) 180.0 (M+H).

### 2.4. Synthesis of methyl 4-propyl-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (300 mg, 1.67 mmol) and allyl-MgBr (3.35 mL, 6.70 mmol; 2.0 M in THF) according to General Procedure 3 to give (E)-methyl 4-allylidene-1,4,5,6-tetrahydrocyclopenta[b]pyrrole-2-carboxylate, followed by General Procedure 6. The crude product was purified by column chromatography (0-40% EtOAc/heptane) to give 99 mg of methyl 4-propyl-1,4,5,6-tetrahydrocyclopenta[b]pyrrole-2-carboxylate as a white solid (29% over two steps). ¹H NMR (400 MHz, METHANOL-d₄) δ ppm 0.95 (t, J=7.03 Hz, 3H), 1.37-1.55 (m, 4H), 1.91-2.03 (m, 1H), 2.53-2.76 (m, 3H), 2.86-2.95 (m, 1H), 3.78 (s, 3H), 6.60 (s, 1H); LCMS-MS (ESI+) 208.0 (M+H).

### 2.5. Synthesis of methyl 4-isopropyl-1,4,5,6-tetrahydrocyclopenta[b]pyrrole-2-carboxylate

### 2.5.a) Synthesis of ethyl 4-(propan-2-ylidene)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (442 mg, 2.47 mmol) and isopropyl magnesium bromide (1M, 9.87 mL, 9.87 mmol, 4 equiv) according to General Procedure 3. Purification by flash chromatography (0-40% EtOAc/heptane) provided 131 mg of a yellow solid (26%). ¹H NMR (400 MHz, CHLOROFORM-d) δ ppm 1.75 (s, 3H) 1.92 (s, 3H) 2.80-2.87 (m, 2H) 2.96-3.03 (m, 2H) 3.85 (s, 3H) 6.86 (d, J=1.76 Hz, 1H) 9.16 (br. s., 1H).

### 2.5.b) Synthesis of methyl 4-isopropyl-1,4,5,6-tetrahydrocyclopenta[b]pyrrole-2-carboxylate

The title compound was synthesized from methyl 4-(propan-2-ylidene)-1,4,5,6-tetrahydrocyclopenta[b]pyrrole-2-carboxylate according to General Procedure 6. Purification by column chromatography (0-100% EtOAc/heptane) gave 91 mg of methyl 4-isopropyl-1,4,5,6-tetrahydrocyclopenta[b]pyrrole-2-carboxylate as a white solid (66%). ¹H NMR (400 MHz, METHANOL-d₄) δ ppm 0.93 (d, J=1.37 Hz, 3H) 0.94 (d, J=1.37 Hz, 3H) 1.61-1.72 (m, 1H) 2.04-2.14 (m, J=12.76, 8.91, 5.66, 5.66 Hz, 1H) 2.45-2.55 (m, 1H) 2.56-2.80 (m, 3H) 3.78 (s, 3H) 6.63 (s, 1H); LCMS-MS (ESI+) 208.0 (M+H).

### 2.6. Synthesis of methyl 4-isobutyl-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (300 mg, 1.68 mmol) and isobutyl-MgBr (4 equiv.) according to General Procedure 3 to give (E)-methyl 4-(2-methylpropylidene)-1,4,5,6-tetrahydrocyclopenta[b]pyrrole-2-carboxylate, followed by General Procedure 6. Purification by column chromatography (0-40% EtOAc/heptane) gave 99 mg of methyl 4-isobutyl-1,4,5,6-tetrahydrocyclopenta[b]pyrrole-2-carboxylate as a white solid (29% over two steps). ¹H NMR (400 MHz, METHANOL-d₄) δ ppm 0.94 (d, J=6.54 Hz, 3H), 0.98 (d, J=6.59 Hz, 3H), 1.22-1.32 (m, 1H), 1.39-1.47 (m, 1H), 1.71-1.83 (m, 1H), 1.89-2.00 (m, 1H), 2.51-2.76 (m, 3H), 2.95-3.04 (m, 1H), 3.78 (s, 3H), 6.59 (s, 1H); LCMS-MS (ESI+) 224.0 (M+H).

### 2.7. Synthesis of methyl 4-(cyclohexylmethyl)-1,4,5,6-tetrahydrocyclopenta[b]pyrrole-2-carboxylate

A solution of cyclohexyl-MgBr (41.56 mL, 10.39 mmol, 0.25 M in THF, 4 eqv.) was added to a stirred solution of 4-oxo-1,4,5,6-tetrahydro-cyclopenta[b]pyrrole-2-carboxylic acid methyl ester (465 mg, 2.59 mmol) in THF (15 mL) at 0° C. under nitrogen. The cold bath was removed and the resulting solution was heated to 67° C. for about 12 h. The reaction was then cooled to rt and quenched with a saturated solution of NH₄Cl before extraction with EtOAc (3×50 mL). The combined extracts were washed with brine, dried (Na₂SO₄), filtered through a plug of silica gel and concentrated. The crude product was subjected to hydrogenation with 10% Pd—C and hydrogen at rt for 4 h in methanol. The catalyst was removed by filtration over Celite and the filtrate concentrated onto silica gel. Purification by column chromatography (0-100% EtOAc/heptane) gave a white solid (90 mg, 13%). ¹H-NMR (400 MHz, CDCl₃) δ ppm 0.85-1.00 (m, 2H) 1.11-1.36 (m, 5H) 1.38-1.51 (m, 2H) 1.64-1.78 (m, 3H) 1.81-1.91 (m, 1H) 1.92-2.03 (m, 1H) 2.53-2.80 (m, 3H) 3.00-3.12 (m, 1H) 3.83 (s, 3H) 6.67 (d, J=1.46 Hz, 1H) 8.89 (br. s., 1H). LCMS-MS (ESI+) 262.0 (M+H).

### 2.8. Synthesis of methyl 4-(4-fluorobenzyl)-1,4,5,6-tetrahydrocyclopenta[b]pyrrole-2-carboxylate

### 2.8.a) Synthesis of (E/Z)-methyl 4-(4-fluorobenzylidene)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (0.4 g, 2.2 mmol) and 4-fluorobenzyl triphenylphosphonium chloride salt (1.09 g, 2.7 mmol) according to General Procedure 2. The crude product was purified by flash chromatography (0-50% EtOAc/heptane) to afford 31.1 mg of (E/Z)-methyl 4-(4-fluorobenzylidene)-1,4,5,6-tetrahydrocyclopenta[b]pyrrole-2-carboxylate in 5% yield. ¹H NMR (400 MHz, CDCl₃) δ ppm 8.93 (br. s., 1H), 7.43-7.50 (m, 2H), 7.04-7.09 (m, 2H), 6.89 (d, J=1.61 Hz, 1H), 6.47 (t, J=2.03 Hz, 1H), 3.36 (td, J=5.79, 2.55 Hz, 2H), 2.93-2.99 (m, 2H); ¹⁹F NMR (376 MHz, CDCl₃) δ ppm −116.03 (s, 1 F).

### 2.8.b) Synthesis of methyl-4-(4-fluorobenzyl)-1,4,5,6-tetrahydrocyclopenta[b]pyrrole-2-carboxylate

Methyl 4-(4-fluorobenzyl)-1,4,5,6-tetrahydrocyclopenta[b]pyrrole-2-carboxylate can be synthesized from (E/Z)-methyl 4-(4-fluorobenzylidene)-1,4,5,6-tetrahydrocyclopenta[b]pyrrole-2-carboxylate according to General Procedure 6.

### 2.9. Syntheses of methyl 4-benzyl-1,4,5,6-tetrahydrocyclopenta[b]pyrrole-2-carboxylate and methyl 4-(4-chlorobenzyl)-1,4,5,6-tetrahydrocyclopenta[b]pyrrole-2-carboxylate

### 2.9.a) Synthesis of ethyl 4-(4-chlorobenzylidene)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (500 mg, 2.79 mmol) and (4-chlorobenzyl)-triphenylphosphonium chloride (1.54 g, 3.63 mmol) according to General Procedure 2. The crude product was purified by flash chromatography (0-20% EtOAc/Heptane) to give 152 mg of methyl 4-(4-chlorobenzylidene)-1,4,5,6-tetrahydrocyclopenta[b]pyrrole-2-carboxylate (20%). ¹H NMR (400 MHz, CDCl₃) δ ppm 2.97 (m, 2H), 3.37 (m, 2H), 3.87 (s, 3H), 6.46 (t, J=2.30 Hz, 1H), 6.89 (d, J=1.71 Hz, 1H), 7.29 (m, 4H), 8.93 (br s, 1H).

### 2.9.b) Synthesis of methyl 4-benzyl-1,4,5,6-tetrahydrocyclopenta[b]pyrrole-2-carboxylate and methyl 4-(4-chlorobenzyl)-1,4,5,6-tetrahydrocyclopenta[b]pyrrole-2-carboxylate

The title compounds were synthesized from methyl 4-(4-chlorobenzylidene)-1,4,5,6-tetrahydrocyclopenta[b]pyrrole-2-carboxylate according to General Procedure 6. Purification by reverse-phase chromatography (75:25 MeOH:water) provided the 4-chloro product (peak 2) in addition to a dehalogenated benzyl adduct (peak 1) in a 4:1 ratio:

Methyl 4-(4-chlorobenzyl)-1,4,5,6-tetrahydrocyclopenta[b]pyrrole-2-carboxylate: (85 mg, 58.2%) as a yellow solid; ¹H NMR (400 MHz, CHLOROFORM-d) δ ppm 2.10 (m, 1H), 2.58 (m, 1H), 2.66 (m, 2H), 2.77 (dd, J=7.10, 1.95 Hz, 2H), 3.28 (m, 1H), 3.81 (s, 3H), 6.37 (d, J=1.56 Hz, 1H), 7.12 (m, 2H), 7.25-7.29 (m, 2H), 8.69 (br s, 1H); LCMS-MS (ESI+) 312.0 (M+Na).

Methyl 4-benzyl-1,4,5,6-tetrahydrocyclopenta[b]pyrrole-2-carboxylate: (28 mg, 21.8%) as a yellow solid; ¹H NMR (400 MHz, CHLOROFORM-d) δ ppm 2.07-2.17 (m, 1H), 2.52-2.63 (m, 1H), 2.63-2.70 (m, 2H), 2.81 (dd, J=7.38, 2.46 Hz, 2H), 3.27-3.36 (m, 1H), 3.80 (s, 3H), 6.39 (d, J=1.66 Hz, 1H), 7.18-7.25 (m, 3H), 7.28-7.33 (m, 2H), 8.75 (br s, 1H); LCMS-MS (ESI+) 278.2 (M+Na).

### 2.10. Synthesis of methyl 4-phenethyl-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate (400 mg, 2.23 mmol) and phenethyl-MgBr (17.9 mL, 8.93 mmol; 0.5 M in THF) according to General Procedure 3 to give (E)-methyl 4-(2-phenylethylidene)-1,4,5,6-tetrahydrocyclopenta[b]pyrrole-2-carboxylate, followed by General Procedure 6. The crude product was purified by column chromatography (0-25% EtOAc/heptane) to give 366 mg of methyl 4-phenethyl-1,4,5,6-tetrahydrocyclopenta-[b]pyrrole-2-carboxylate as a white solid (61% over two steps). ¹H NMR (400 MHz, Methanol-d₄) δ ppm 1.68-1.87 (m, 2H), 1.98-2.09 (m, 1H), 2.55-2.79 (m, 5H), 2.88-2.98 (m, 1H), 3.79 (s, 3H), 6.68 (s, 1H), 7.10-7.32 (m, 5H); LCMS-MS (ESI+) 292.0 (M+Na).

### 2.11. Synthesis of 4-oxo-1,4,5,6-tetrahydrocyclopenta[b]pyrrole-2-carboxylic acid (2)

The title compound was synthesized from methyl 4-oxo-1,4,5,6-tetrahydrocyclopenta[b]pyrrole-2-carboxylate (11 mg, 0.06 mmol) and lithium hydroxide monohydrate (10 mg, 0.25 mmol) according to General Procedure 7 (6.8 mg, 67%). ¹H NMR (400 MHz, METHANOL-d₄) δ ppm 2.89-2.93 (m, 2H), 2.98-3.02 (m, 2H), 6.89 (s, 1H); LCMS-MS (ESI+) 163.7 (M−H); HPLC (UV=100%).

### 2.12. Synthesis of 3-tert-butyl-4-oxo-1,4,5,6-tetrahydrocyclopenta[b]pyrrole-2-carboxylic acid (3)

The title compound was synthesized from methyl 3-tert-butyl-4-oxo-1,4,5,6-tetrahydrocyclopenta[b]pyrrole-2-carboxylate (33 mg, 0.14 mmol) and lithium hydroxide monohydrate (30 mg, 0.71 mmol) according to General Procedure 7. Purification by reverse phase semi-preperative HPLC provided a pure fraction (11.6 mg, 37%) of 3-tert-butyl-4-oxo-1,4,5,6-tetrahydrocyclopenta[b]pyrrole-2-carboxylic acid (3). ¹H NMR (400 MHz, METHANOL-d₄) δ ppm 1.46 (s, 9H), 2.82-2.90 (m, 4H); LCMS-MS (ESI+) 221.7 (M−H); HPLC (UV=95.8%).

### 2.13. Synthesis of 4-methyl-1,4,5,6-tetrahydrocyclopenta[b]pyrrole-2-carboxylic acid (4)

The title compound was synthesized from methyl 4-methyl-1,4,5,6-tetrahydrocyclopenta[b]pyrrole-2-carboxylate and lithium hydroxide monohydrate (17 mg, 0.40 mmol) according to General Procedure 7. The crude product was dried onto Silica gel and was purified by flash chromatography (0-80% EtOAc/Heptane) to give 8.6 mg of 4-methyl-1,4,5,6-tetrahydrocyclopenta[b]pyrrole-2-carboxylic acid (4) as a light yellow solid (52%). ¹H NMR (400 MHz, methanol-d₄) δ ppm 1.17 (d, J=6.78 Hz, 3H), 1.85-1.96 (m, 1H), 2.56-2.77 (m, 3H), 2.95-3.06 (m, 1H), 6.59 (s, 1H); LCMS-MS (ESI+) 166.0 (M+H); HPLC (UV=100%).

The enantiomers of 4-methyl-1,4,5,6-tetrahydrocyclopenta[b]pyrrole-2-carboxylic acid were separated according to General Procedure 8 with 20% methanol in CO₂ with 0.2% diethylamine to give 4-methyl-1,4,5,6-tetrahydrocyclopenta[b]pyrrole-2-carboxylic acid (4), (peak 2, retention time=9.7 min; 96% ee) and 4-methyl-1,4,5,6-tetrahydrocyclopenta[b]pyrrole-2-carboxylic acid (6) (peak 1, retention time=8.1 min; 100% ee).

### 2.14. Synthesis of 4-propyl-1,4,5,6-tetrahydrocyclopenta[b]pyrrole-2-carboxylic acid (7)

The title compound was synthesized from methyl 4-propyl-1,4,5,6-tetrahydrocyclopenta[b]pyrrole-2-carboxylate (95 mg, 0.46 mmol) and lithium hydroxide monohydrate (77 mg, 1.83 mmol) according to General Procedure 7. The crude product was dried onto Silica gel and was purified by flash chromatography (0-80% EtOAc/Heptane) to give 70 mg of 4-propyl-1,4,5,6-tetrahydrocyclopenta[b]pyrrole-2-carboxylic acid (7) as a light yellow solid (79%). ¹H NMR (400 MHz, methanol-d₄) δ ppm 0.96 (t, J=7.03 Hz, 3H), 1.36-1.56 (m, 4H), 1.92-2.03 (m, 1H), 2.52-2.76 (m, 3H), 2.87-2.96 (m, 1H), 6.61 (s, 1H); LCMS-MS (ESI−) 192.2 (M−H); HPLC (UV=100%).

The enantiomers of 4-propyl-1,4,5,6-tetrahydrocyclopenta[b]pyrrole-2-carboxylic acid were separated according to General Procedure 8 using 40% methanol in CO₂ with 0.05% diethylamine to give 4-propyl-1,4,5,6-tetrahydrocyclopenta[b]pyrrole-2-carboxylic acid (9) (peak 1, retention time=3.5 min; 100% ee) and 4-propyl-1,4,5,6-tetrahydrocyclopenta[b]pyrrole-2-carboxylic acid (8) (peak 2, retention time=6.3 min; 100% ee).

### 2.15. Synthesis of 4-isopropyl-1,4,5,6-tetrahydrocyclopenta[b]pyrrole-2-carboxylic acid (10)

The title compound was synthesized from methyl 4-isopropyl-1,4,5,6-tetrahydrocyclopenta[b]pyrrole-2-carboxylate (0.09 g, 0.43 mmol) and lithium hydroxide monohydrate (185 mg, 4.3 mmol) according to General Procedure 7. The crude product was dried onto Silica gel and was purified by chromatography (0 to 100% EtOAc/heptane) to give 4-isopropyl-1,4,5,6-tetrahydrocyclopenta[b]pyrrole-2-carboxylic acid (10) as brown solid (0.018 g, 21%). ¹H NMR (400 MHz, METHANOL-d₄) δ ppm 0.93 (d, J=2.68 Hz, 3H) 0.95 (d, J=2.68 Hz, 3H) 1.67 (dq, J=13.37, 6.67 Hz, 1H) 2.04-2.14 (m, J=12.81, 8.91, 5.71, 5.71 Hz, 1H) 2.44-2.55 (m, 1H) 2.56-2.80 (m, 3H) 6.63 (s, 1H). LCMS m/e 194 (M+H). 92.0% pure by HPLC.

### 2.16. Synthesis of 4-isobutyl-1,4,5,6-tetrahydrocyclopenta[b]pyrrole-2-carboxylic acid (11)

The title compound was synthesized from methyl 4-isobutyl-1,4,5,6-tetrahydrocyclopenta[b]pyrrole-2-carboxylate (37 mg, 0.17 mmol) and lithium hydroxide monohydrate (28 mg, 0.67 mmol) according to General Procedure 7. The crude product was dried onto Silica gel and was purified by flash chromatography (0-80% EtOAc/Heptane) to give 4-isobutyl-1,4,5,6-tetrahydrocyclopenta[b]pyrrole-2-carboxylic acid (11) as a light yellow solid (22 mg, 63%). ¹H NMR (400 MHz, methanol-d₄) δ ppm 0.94 (d, J=6.62 Hz, 3H), 0.98 (d, J=6.59 Hz, 3H), 1.23-1.32 (m, 1H), 1.40-1.48 (m, 1H), 1.73-1.84 (m, 1H), 1.89-2.01 (m, 1H), 2.53-2.76 (m, 3H), 2.95-3.05 (m, 1H), 6.59 (s, 1H); LCMS-MS (ESI−) 206.2 (M−H); HPLC (UV=100%).

### 2.17. Synthesis of 4-(cyclohexylmethyl)-1,4,5,6-tetrahydrocyclopenta[b]pyrrole-2-carboxylic acid (12)

The title compound was synthesized from methyl 4-(cyclohexylmethyl)-1,4,5,6-tetrahydrocyclopenta[b]pyrrole-2-carboxylate and lithium hydroxide monohydrate according to General Procedure 7. The crude product was dried onto silica gel and purified by chromatography (0 to 100% EtOAc/heptane) to give 4-(cyclohexylmethyl)-1,4,5,6-tetrahydrocyclopenta[b]pyrrole-2-carboxylate as a brown solid (0.018 g, 21%). ¹H NMR (400 MHz, methanol-d₄) δ ppm 0.87-1.02 (m, 2H) 1.14-1.36 (m, 5H) 1.36-1.54 (m, 2H) 1.63-1.80 (m, 3H) 1.83-2.00 (m, 2H) 2.50-2.77 (m, 3H) 2.97-3.09 (m, 1H) 6.59 (s, 1H). LCMS m/e 248 (M+H). 97.5% pure by HPLC.

### 2.18. Synthesis of (E)-4-(4-fluorobenzylidene)-1,4,5,6-tetrahydrocyclopenta[b]pyrrole-2-carboxylic acid (13)

The title compound was synthesized from (E/Z)-methyl 4-(4-fluorobenzylidene)-1,4,5,6-tetrahydrocyclopenta[b]pyrrole-2-carboxylate (2.4 mg, 0.0088 mmol) and sodium hydroxide according to General Procedure 7. The crude product was purified by preparative HPLC (40%-100% methanol/water with 0.1% formic acid and 1% acetonitrile) to afford a 1:1 mixture of (E)- and (Z)-4-(4-fluorobenzylidene)-1,4,5,6-tetrahydrocyclopenta[b]pyrrole-2-carboxylic acid (13) (0.9 mg, 39%). 98.5% (HPLC, UV). LCMS m/e 258 (M+H); 256 (M−H). ¹H NMR (400 MHz, methanol-d₄) δ ppm 8.50 (br. s., 1H), 7.33-7.43 (m, 2H), 6.98-7.07 (m, 2H), 6.78 (s, 1H), 6.42 (t, J=2.22 Hz, 1H), 3.15-3.20 (m, 1H), 2.89-2.96 (m, 2H), 2.80-2.86 (m, 1H). ¹⁹F NMR (376 MHz, methanol-d₄) δ ppm −156.24 (s, 1 F).

The title compound can be converted to 4-(4-fluorobenzyl)-1,4,5,6-tetrahydrocyclopenta[b]pyrrole-2-carboxylic acid using General Procedure 6.

### 2.19. Synthesis of 4-(4-chlorobenzyl)-1,4,5,6-tetrahydrocyclopenta[b]pyrrole-2-carboxylic acid (14)

The title compound was synthesized from methyl 4-(4-chlorobenzyl)-1,4,5,6-tetrahydrocyclopenta[b]pyrrole-2-carboxylate (85 mg, 0.293 mmol) and lithium hydroxide monohydrate (67 mg, 1.58 mmol) according to General Procedure 7. The crude product was purified by flash chromatography (0-80% EtOAc/Heptane) to give 4-(4-chlorobenzyl)-1,4,5,6-tetrahydrocyclopenta[b]pyrrole-2-carboxylic acid (14) as a light red solid (60 mg, 69%). ¹H NMR (400 MHz, METHANOL-d₄) δ ppm 2.04-2.14 (m, 1H), 2.42-2.59 (m, 1H), 2.59-2.65 (m, 2H), 2.72 (dd, J=13.37, 7.91, 1H), 2.78 (dd, J=13.37, 6.83, 1H), 3.21-3.29 (m, 1H), 6.29 (s, 1H), 7.14-7.19 (m, 2H), 7.24-7.28 (m, 2H); LCMS-MS (ESI+) 274.2 (M−H); HPLC (UV=100%).

The enantiomers of 4-(4-chlorobenzyl)-1,4,5,6-tetrahydrocyclopenta[b]pyrrole-2-carboxylic acid were separated according to General Procedure 8 using 50% methanol in CO₂ with 0.05% diethylamine to give 4-(4-chlorobenzyl)-1,4,5,6-tetrahydrocyclopenta[b]pyrrole-2-carboxylic acid (16) (retention time=4.5 min; 100% ee) and 4-(4-chlorobenzyl)-1,4,5,6-tetrahydrocyclopenta[b]pyrrole-2-carboxylic acid (15) (retention time=6.9 min; 98.5% ee).

### 2.20. Synthesis of 4-benzyl-1,4,5,6-tetrahydrocyclopenta[b]pyrrole-2-carboxylic acid (17)

The title compound was synthesized from methyl 4-benzyl-1,4,5,6-tetrahydrocyclopenta[b]pyrrole-2-carboxylate (25 mg, 0.098 mmol) and lithium hydroxide monohydrate (25 mg, 0.39 mmol) according to General Procedure 7. The crude product was dried onto Silica gel and was purified by flash chromatography (0-80% EtOAc/Heptane) to give 4-benzyl-1,4,5,6-tetrahydrocyclopenta[b]pyrrole-2-carboxylic acid (17) as a light red solid (19 mg, 81%). ¹H NMR (400 MHz, METHANOL-d₄) δ ppm 2.05-2.14 (m, 1H), 2.48-2.58 (m, 1H), 2.59-2.66 (m, 2H), 2.74 (dd, J=13.32, 7.81, 1H), 2.79 (dd, J=13.32, 6.98, 1H), 3.22-3.30 (m, 1H), 6.29 (s, 1H), 7.15-7.21 (m, 3H), 7.24-7.29 (m, 2H); LCMS-MS (ESI+) 240.2 (M−H); HPLC (UV=100%).

### 2.21. Synthesis of 4-phenethyl-1,4,5,6-tetrahydrocyclopenta[b]pyrrole-2-carboxylic acid (18)

The title compound was synthesized from methyl 4-phenethyl-1,4,5,6-tetrahydrocyclopenta[b]pyrrole-2-carboxylate and lithium hydroxide monohydrate (81 mg, 1.93 mmol) according to General Procedure 7. Silica gel was added, the solvent stripped off and the silica gel-imbedded material was purified by flash chromatography (0-80% EtOAc/Heptane) to give 4-phenethyl-1,4,5,6-tetrahydrocyclopenta[b]pyrrole-2-carboxylic acid (18) as a light yellow solid (103.6 mg, 84%). ¹H NMR (400 MHz, methanol-d₄) δ ppm 1.68-1.87 (m, 2H), 1.98-2.09 (m, 1H), 2.55-2.80 (m, 5H), 2.88-2.97 (m, 1H), 6.68 (s, 1H), 7.11-7.16 (m, 1H), 7.17-7.29 (m, 4H); LCMS-MS (ESI+) 256.0 (M+H); HPLC (UV=100%), (ELSD=100%).

The enantiomers of 4-phenethyl-1,4,5,6-tetrahydrocyclopenta[b]pyrrole-2-carboxylic acid were separated according to General Procedure 8 with 20% of a mixture of 50:50 methanol/isopropanol in CO₂ with 0.2% diethylamine to give 4-phenethyl-1,4,5,6-tetrahydrocyclopenta[b]pyrrole-2-carboxylic acid (19) (peak 1, retention time=12.8 min; 100% ee) and 4-phenethyl-1,4,5,6-tetrahydrocyclopenta[b]pyrrole-2-carboxylic acid (20) (peak 2, retention time=13.8 min; 95% ee).

### 2.22. Synthesis of methyl 4-(4-isopropylbenzyl)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate in two steps. First, methyl 4-oxo-1,4,5,6-tetrahydrocyclopenta[b]pyrrole-2-carboxylate (0.3 g, 1.7 mmol, 1 equiv) was reacted with 4-4-isopropylbenzyl-MgCl (26.8 mL, 0.25M in THF, 6.7 mmol, 4 equiv) according to General Procedure 3. The resulting olefin was converted to the title compound according to General Procedure 6. Purification by preparative HPLC (Chromeleon purification system, 0.1% formic acid/1% acetonitrile mixture in water with methanol, 50 mm Dynamax HPLC C-18 column, 28 mL/min; 80-100% methanol) gave methyl 4-(4-isopropylbenzyl)-1,4,5,6-tetrahydrocyclopenta[b]pyrrole-2-carboxylate (17.7 mg). ¹H NMR (400 MHz, CHLOROFORM-d) δ ppm 8.69 (br. s., 1H), 7.10-7.20 (m, 4H), 6.44 (d, J=1.42 Hz, 1H), 3.81 (s, 3H), 3.23-3.35 (m, 1H), 2.50-2.97 (m, 6H), 2.05-2.19 (m, 1H), 1.27 (d, J=6.91 Hz, 6H).

### 2.23. Synthesis of 4-(4-isopropylbenzyl)-1,4,5,6-tetrahydrocyclopenta[b]pyrrole-2-carboxylic acid (33)

The title compound was synthesized from methyl 4-(4-isopropylbenzyl)-1,4,5,6-tetrahydrocyclopenta[b]pyrrole-2-carboxylate and lithium hydroxide monohydrate according to General Procedure 7. Silica gel was added, the solvent stripped off and the silica gel-imbedded material was purified by flash chromatography (0-80% EtOAc/Heptane) to give 4-(4-isopropylbenzyl)-1,4,5,6-tetrahydrocyclopenta[b]pyrrole-2-carboxylic acid (33) as a light yellow solid.

### 2.24. Synthesis of methyl 4-(4-methoxyphenethyl)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate in two steps. First, methyl 4-oxo-1,4,5,6-tetrahydrocyclopenta[b]pyrrole-2-carboxylate (300 mg, 1.67 mmol) was reacted with 4-methoxyphenethyl-MgCl (13.4 mL, 6.70 mmol; 0.5 M in THF) according to General Procedure 3. The resulting olefin was converted to the title compound according to General Procedure 6. The crude product was purified by column chromatography (0-40% EtOAc/heptane) to give methyl 4-(4-methoxyphenethyl)-1,4,5,6-tetrahydrocyclopenta[b]pyrrole-2-carboxylate (240 mg, 48% over two steps). ¹H NMR (400 MHz, methanol-d₄) δ ppm 1.64-1.87 (m, 2H), 1.96-2.07 (m, 1H), 2.51-2.77 (m, 5H), 2.87-2.96 (m, 1H), 3.75 (s, 3H), 3.78 (s, 3H), 6.66 (s, 1H), 6.78-6.83 (m, 2H) 7.06-7.12 (m, 2H); LCMS-MS (ESI+) 322.2 (M+Na).

### 2.25. Synthesis of 4-(4-methoxyphenethyl)-1,4,5,6-tetrahydrocyclopenta[b]pyrrole-2-carboxylic acid (34)

The title compound was synthesized from methyl 4-(4-methoxyphenethyl)-1,4,5,6-tetrahydrocyclopenta[b]pyrrole-2-carboxylate (235 mg, 0.78 mmol) and lithium hydroxide monohydrate (132 mg, 3.14 mmol) according to General Procedure 7. Silica gel was added, the solvent stripped off and the silica gel-imbedded material was purified by flash chromatography (0-80% EtOAc/Heptane) to give 4-(4-methoxyphenethyl)-1,4,5,6-tetrahydrocyclopenta[b]pyrrole-2-carboxylic acid (34) as a light yellow solid (142 mg, 63%). ¹H NMR (400 MHz, methanol-d₄) δ ppm 1.64-1.84 (m, 2H), 1.96-2.08 (m, 1H), 2.53-2.78 (m, 5H), 2.85-2.97 (m, 1H), 3.75 (s, 3H), 6.67 (s, 1H), 6.77-6.86 (m, 2H), 7.06-7.16 (m, 2H); LCMS-MS (ESI+) 286.1 (M+H); HPLC (UV=100%), (ELSD=100%).

### 2.26. Synthesis of methyl 4-(2-methyl-2-phenylpropyl)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate in two steps. First, methyl 4-oxo-1,4,5,6-tetrahydrocyclopenta[b]pyrrole-2-carboxylate (420 mg, 2.34 mmol) was reacted with (2-methyl-2-phenylpropyl)-MgCl (19 mL, 9.38 mmol, 0.5 M in THF, 4 equiv) according to General Procedure 3. The resulting olefin was converted to the title compound according to General Procedure 6. Purification by column chromatography (Isco CombiFlash), eluting with a gradient of 0-100% EtOAc/heptane, gave methyl 4-(2-methyl-2-phenylpropyl)-1,4,5,6-tetrahydrocyclopenta[b]pyrrole-2-carboxylate as a white solid (50 mg, 7.2%). ¹H NMR (400 MHz, CHLOROFORM-d) δ ppm 1.40 (s, 3H) 1.42 (s, 3H) 1.73-1.85 (m, 2H) 2.15 (dd, J=14.11, 4.00 Hz, 1H) 2.29 (dt, J=9.27, 7.76 Hz, 1H) 2.47-2.65 (m, 2H) 2.73-2.82 (m, 1H) 3.81 (s, 3H) 6.47 (d, J=1.07 Hz, 0H) 7.22-7.27 (m, 1H) 7.31-7.36 (m, 2H) 7.39-7.44 (m, 2H) 8.77 (br. s, 1H). LCMS-MS (ESI+) 298.0 (M+H).

### 2.27. Synthesis of 4-(2-methyl-2-phenylpropyl)-1,4,5,6-tetrahydrocyclopenta[b]pyrrole-2-carboxylic acid (35)

The title compound was synthesized from methyl 4-(2-methyl-2-phenylpropyl)-1,4,5,6-tetrahydrocyclopenta[b]pyrrole-2-carboxylate (0.10 g, 0.33 mmol) and lithium hydroxide monohydrate (142 mg, 3.3 mmol) according to General Procedure 7. Silica gel was added, the solvent stripped off and the silica gel-imbedded material was purified by flash chromatography (0-100% EtOAc/Heptane) to give 4-(2-methyl-2-phenylpropyl)-1,4,5,6-tetrahydrocyclopenta[b]pyrrole-2-carboxylic acid (35) as a reddish brown solid (2.4 mg). ¹H NMR (400 MHz, methanol-d₄) δ ppm 1.38 (s, 3H) 1.41 (s, 3H) 1.69-1.83 (m, 2H) 2.13 (dd, J=14.06, 3.86 Hz, 1H) 2.20-2.30 (m, 1H) 2.42-2.62 (m, 2H) 2.66-2.77 (m, 1H) 6.40 (s, 1H) 7.13-7.19 (m, 1H) 7.30 (t, J=7.79 Hz, 2H) 7.39-7.45 (m, 2H) 8.48 (s, 1H). LCMS m/e 284 (M+H). 97.9% pure by HPLC.

### 2.28. Synthesis of methyl 4-(3-phenylpropyl)-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate in two steps. First, methyl 4-oxo-1,4,5,6-tetrahydrocyclopenta[b]pyrrole-2-carboxylate (300 mg, 1.67 mmol) was reacted with 3-phenyl-1-propyl-MgBr (13.4 mL, 6.70 mmol; 0.5 M in THF) according to General Procedure 3. The resulting olefin was converted to the title compound according to General Procedure 6. The crude product was purified by column chromatography (0-40% EtOAc/heptane) to afford methyl 4-(3-phenylpropyl)-1,4,5,6-tetrahydrocyclopenta[b]pyrrole-2-carboxylate (212 mg, 45% over two steps). ¹H NMR (400 MHz, methanol-d₄) δ ppm 1.41-1.58 (m, 2H), 1.63-1.83 (m, 2H), 1.90-2.00 (m, 1H), 2.52-2.74 (m, 5H), 2.87-2.96 (m, 1H), 3.77 (s, 3H), 6.60 (s, 1H), 7.11-7.20 (m, 3H) 7.22-7.27 (m, 2H); LCMS-MS (ESI+) 306.2 (M+Na).

### 2.29. Synthesis of 4-(3-phenylpropyl)-1,4,5,6-tetrahydrocyclopenta[b]pyrrole-2-carboxylic acid (36)

The title compound was synthesized from methyl 4-(3-phenylpropyl)-1,4,5,6-tetrahydrocyclopenta[b]pyrrole-2-carboxylate (210 mg, 0.74 mmol) and lithium hydroxide monohydrate (124 mg, 2.96 mmol) according to General Procedure 7. Silica gel was added, the solvent stripped off and the silica gel-imbedded material was purified by flash chromatography (0-80% EtOAc/Heptane) to give 4-(3-phenylpropyl)-1,4,5,6-tetrahydrocyclopenta[b]pyrrole-2-carboxylic acid (36) as a light brown solid (113.8 mg, 57%). ¹H NMR (400 MHz, methanol-d₄) δ ppm 1.42-1.59 (m, 2H), 1.64-1.84 (m, 2H), 1.90-2.01 (m, 1H), 2.52-2.75 (m, 5H), 2.88-2.97 (m, 1H), 6.61 (s, 1H), 7.10-7.20 (m, 3H), 7.21-7.28 (m, 2H); LCMS-MS (ESI+) 270.1 (M+H); HPLC (UV=100%), (ELSD=100%).

### 2.30. Synthesis of methyl 4-p-tolyl-1,4,5,6-tetrahydrocyclopenta[b]pyrrole-2-carboxylate

The title compound was synthesized from methyl 4-oxo-1,4,5,6-tetrahydrocyclopenta[b]pyrrole-2-carboxylate in two steps. First, methyl 4-oxo-1,4,5,6-tetrahydrocyclopenta[b]pyrrole-2-carboxylate (0.3 g, 1.7 mmol) was reacted with p-tolyl-MgBr (6.7 mL, 1 M in THF, 6.7 mmol) according to General Procedure 3. The resulting olefin was converted to the title compound according to General Procedure 6. The desired methyl 4-p-tolyl-1,4,5,6-tetrahydrocyclopenta[b]pyrrole-2-carboxylate was used in the next step without further purification (0.289 g). ¹H NMR (400 MHz, CDCl₃) δ ppm 9.06 (br. s., 1H), 7.11 (s, 4H), 6.65 (d, J=1.59 Hz, 1H), 4.20 (t, J=7.27 Hz, 1H), 3.83 (s, 3H), 2.72-2.98 (m, 3H), 2.34 (s, 3H), 2.23-2.32 (m, 1H).

### 2.31. Synthesis of 4-p-tolyl-1,4,5,6-tetrahydrocyclopenta[b]pyrrole-2-carboxylic acid (37)

The title compound was synthesized from methyl 4-p-tolyl-1,4,5,6-tetrahydrocyclopenta[b]pyrrole-2-carboxylate (0.2888 g, 1.2 mmol, 1 equiv) and sodium hydroxide (2.94 mL, 10 M, 25 equiv) according to General Procedure 7. The resulting product was purified by preparative HPLC (water with 0.1% formic acid and 1% acetonitrile/methanol; 50 mm Dynamax HPLC C-18 column; 28 mL/min; 60% to 100% methanol) to give 4-p-tolyl-1,4,5,6-tetrahydrocyclopenta[b]pyrrole-2-carboxylic acid (37) (76.9 mg, 28%) ¹H NMR (400 MHz, methanol-d₄) δ ppm 7.02-7.10 (m, 4H), 6.50 (s, 1H), 4.13 (t, J=7.20 Hz, 1H), 2.68-2.94 (m, 3H), 2.29 (s, 3H), 2.14-2.25 (m, 1H); LCMS m/e 240 (M−H).

### Example 3

### Synthesis of Pyrrole Analogs with 5-substituted Fused Cyclopentanes

### 3.1. Synthesis of methyl 5,5-difluoro-1,4,5,6-tetrahydrocyclopenta[b]pyrrole-2-carboxylate

### 3.1.a Synthesis of methyl-5,5-difluoro-4-oxo-1,4,5,6-tetrahydrocyclopenta[b]pyrrole-2-carboxylate

To a solution of methyl 4-oxo-1,4,5,6-tetrahydrocyclopenta[b]pyrrole-2-carboxylate (310 mg, 1.73 mmol) in anhydrous 5:1 THF/Et₂O (30 mL) at −78° C. under nitrogen was slowly added a freshly prepared solution of lithium diisopropylamide (0.5M in THF, 12.84 mL, 6.42 mmol). The reaction mixture was stirred at −78° C. for 30 minutes, warmed to −40° C. for a period of 1 h, then cooled again to −78° C. A solution of NFSI (1.20 g, 3.81 mmol, 2.20 eq.) in 3 mL anhydrous THF was added over a period of 15 min while maintaining −78° C. internal temperature. The reaction mixture was kept at −78° C. for 2 h and was then allowed to warm to rt over a period of 12 h. Analysis of the reaction mixture by TLC (9:1 heptane/EtOAc) showed that the reaction had reached completion. Water (10 mL) was carefully added to the reaction mixture followed by 0.5M HCl until the pH was about 2-3. The mixture was extracted with EtOAc (4×100 mL) and the combined extracts were washed with brine, dried (Na₂SO₄) and concentrated onto Celite. The compound was purified by flash chromatography (0-25% EtOAc/heptane) and reverse-phase chromatography (MeCN/H₂O, 0.05% TFA) to afford 111.2 mg of methyl 5,5-difluoro-4-oxo-1,4,5,6-tetrahydrocyclopenta[b]pyrrole-2-carboxylate (31%). ¹H NMR (400 MHz, acetone-d₆) δ ppm 3.59 (t, 2H), 3.87 (s, 3H), 7.01 (s, 1H), 11.89 (bs, 1H); ¹⁹F NMR (400 MHz, Acetone-d₆) δ ppm −107.19; LCMS-MS (ESI+) 216.0 (M+H).

### 3.1.b Synthesis of methyl 5,5-difluoro-1,4,5,6-tetrahydrocyclopenta[b]pyrrole-2-carboxylate

To a solution of methyl 5,5-difluoro-4-oxo-1,4,5,6-tetrahydrocyclopenta[b]pyrrole-2-carboxylate (111.2 mg, 0.517 mmol) in TFA (1 mL) under nitrogen at 25° C. was added triethylsilane (0.25 mL, 1.55 mmol) and the reaction mixture was stirred for 18 h at 25° C. TLC analysis (10% MeOH/DCM) indicated that all starting material had been consumed. The solvent was removed using a nitrogen stream and the residue was taken up in MeCN and purified by reverse-phase chromatography (MeCN/H₂O, 0.05% TFA) to afford 3.9 mg of methyl 5,5-difluoro-1,4,5,6-tetrahydrocyclopenta[b]pyrrole-2-carboxylate in 3.8% yield. ¹H NMR (400 MHz, Acetone-d₆) δ ppm 3.15 (t, 2H), 3.31 (t, 2H), 3.76 (s, 3H), 6.64 (d, 1H), 10.83 (bs, 1H); ¹⁹F NMR (400 MHz, Acetone-d₆) δ ppm −86.73; LCMS-MS (ESI+) 202.0 (M+H).

### 3.1.c Synthesis of 5,5-difluoro-1,4,5,6-tetrahydrocyclopenta[b]pyrrole-2-carboxylic acid

To a solution of methyl 5,5-difluoro-1,4,5,6-tetrahydrocyclopenta[b]pyrrole-2-carboxylate (3.90 mg, 0.019 mmol) in 3 mL of 4:1 EtOH/H₂O was added lithium hydroxide (2.0M, 0.10 mL, 0.194 mmol, 10.0 eq) and heated at 50° C. for 3 h. The solvent was then removed using a nitrogen stream and the residue was taken up in MeCN/H₂O (2:1) and the solvent was removed again using a nitrogen stream. This was repeated once more before the residue was taken up in H₂O (2 mL). The solution was acidified to pH 4 with dilute HCl. The material was purified by reverse-phase chromatography (MeCN/H₂O, 0.05% TFA) to afford 1.6 mg of 5,5-difluoro-1,4,5,6-tetrahydrocyclopenta[b]pyrrole-2-carboxylic acid in 45.0% yield. ¹H NMR (400 MHz, acetone-d₆) δ ppm 3.16 (t, 2H), 3.31 (t, 2H), 3.76 (s, 3H), 6.66 (d, 1H), 10.75 (bs, 1H); ¹⁹F NMR (400 MHz, acetone-d₆) δ ppm −86.73; LCMS-MS (ESI+) 188.0 (M+H).

### 3.2. Synthesis of methyl 5-methyl-1,4,5,6-tetrahydrocyclopenta[b]pyrrole-2-carboxylate

3.2.a) Synthesis of 1-tert-butyl 2-methyl 4-oxo-5,6-dihydrocyclopenta[b]pyrrole-1,2(4H)-dicarboxylate

4-(Dimethylamino)pyridine (0.062 g, 0.51 mmol) was added to a stirred solution of methyl 4-oxo-1,4,5,6-tetrahydrocyclopenta[b]pyrrole-2-carboxylate (1.00 g, 5.58 mmol) in CH₂Cl₂ (DCM) (20 mL) at rt under nitrogen. A solution of di-tert-butyl dicarbonate (1.33 g, 6.09 mmol) in CH₂Cl₂ (10 mL) was then added and the solution was stirred for 18 h. The reaction was quenched with a saturated solution of NH₄Cl before extraction with EtOAc (3×50 mL). The combined extracts were washed with brine and dried over Na₂SO₄. Purification by column chromatography (0-50% EtOAc/heptane) gave the title compound as a clear, yellow oil (1.38 g, 88%). R_{f} (1:1 EtOAc/heptane)=0.40; ¹H NMR (400 MHz, CDCl₃-d) δ ppm 1.62 (s, 9H), 2.90-2.92 (m, 2H), 3.14-3.17 (m, 2H), 3.88 (s, 3H), 6.90 (s, 1H).

### 3.2.b) Synthesis of 1-tert-butyl 2-methyl 5-methyl-4-oxo-5,6-dihydrocyclopenta[b]pyrrole-1,2(4H)-dicarboxylate and methyl 5-methyl-4-oxo-1,4,5,6-tetrahydrocyclopenta[b]pyrrole-2-carboxylate

The title compound was synthesized from 1-tert-butyl 2-methyl 4-oxo-5,6-dihydrocyclopenta[b]pyrrole-1,2(4H)-dicarboxylate (0.53 g, 1.9 mmol) and iodomethane (0.297 g, 1.9 mmol) according to General Procedure 4. Purification of the resulting mixture by column chromatography (0-100% EtOAc/heptane), yielded both the BOC-protected product 1-tert-butyl 2-methyl 5-methyl-4-oxo-5,6-dihydrocyclopenta[b]pyrrole-1,2(4H)-dicarboxylate and the deprotected methyl 5-methyl-4-oxo-1,4,5,6-tetrahydrocyclopenta[b]pyrrole-2-carboxylate:

1-tert-butyl 2-methyl 5-methyl-4-oxo-5,6-dihydrocyclopenta[b]pyrrole-1,2(4H)-dicarboxylate was isolated as an orange solid (0.073 g, 13%). R_{f} (1:1 EtOAc:heptane)=0.54; ¹H NMR (400 MHz, CHLOROFORM-d) δ ppm 1.34 (d, J=7.6 Hz, 3H), 1.62 (s, 9H), 2.65-2.75 (m, 1H), 2.92-3.01 (m, 1H), 3.34-3.45 (m, 1H), 3.88 (s, 3H), 6.90 (s, 1H); LCMS-MS (ESI+) 294.1 (M+H).

Methyl 5-methyl-4-oxo-1,4,5,6-tetrahydrocyclopenta[b]pyrrole-2-carboxylate was isolated as an orange solid (0.045 g, 12%). R_{f} (1:1 EtOAc:heptane)=0.26; ¹H NMR (400 MHz, CDCl₃) δ ppm 1.34 (d, J=7.5 Hz, 3H), 2.56-2.61 (m, 1H), 2.98-3.06 (m, 1H), 3.21-3.27 (m, 1H), 3.89 (s, 3H), 6.98 (s, 1H).

### 3.2.c) Synthesis of ethyl 5-methyl-1,4,5,6-tetrahydrocyclopenta[b]pyrrole-2-carboxylate

The title compound was synthesized from 1-tert-butyl 2-methyl 5-methyl-4-oxo-5,6-dihydrocyclopenta[b]pyrrole-1,2(4H)-dicarboxylate (0.053 g, 0.181 mmol) according to General Procedure 5. Purification by column chromatography (0-30% EtOAc/heptane) yielded methyl 5-methyl-1,4,5,6-tetrahydrocyclopenta[b]pyrrole-2-carboxylate as an off-white solid (0.004 g, 11% yield). R_{f} (1:1 EtOAc/heptane)=0.64; ¹H NMR (400 MHz, CDCl₃) δ ppm 1.21 (d, J=6.7 Hz, 3H), 2.20-2.36 (m, 2H), 2.70-3.00 (m, 3H), 3.82 (s, 3H), 6.64 (s, 1H); LCMS-MS (ESI+) 180.1 (M+H).

### 3.2.d) Synthesis of ethyl 5-methyl-1,4,5,6-tetrahydrocyclopenta[b]pyrrole-2-carboxylate

The title compound was synthesized from methyl 5-methyl-4-oxo-1,4,5,6-tetrahydrocyclopenta[b]pyrrole-2-carboxylate (0.045 g, 0.233 mmol) according to General Procedure 5. Purification by column chromatography (0-30% EtOAc/heptane) yielded methyl 5-methyl-1,4,5,6-tetrahydrocyclopenta[b]pyrrole-2-carboxylate as an off-white solid (0.013 g, 31% yield). R_{f} (1:1 EtOAc:heptane)=0.64; ¹H NMR (400 MHz, CHLOROFORM-d) δ ppm 1.21 (d, J=6.7 Hz, 3H), 2.20-2.36 (m, 2H), 2.70-3.00 (m, 3H), 3.82 (s, 3H), 6.64 (s, 1H); LCMS-MS (ESI+) 180.1 (M+H).

### 3.3. Synthesis of methyl 5-benzyl-1,4,5,6-tetrahydrocyclopenta[b]pyrrole-2-carboxylate

### 3.3.a) Synthesis of 1-tert-butyl 2-methyl 5-benzyl-4-oxo-5,6-dihydrocyclopenta[b]pyrrole-1,2(4H)-dicarboxylate and methyl 5-benzyl-4-oxo-1,4,5,6-tetrahydrocyclopenta[b]pyrrole-2-carboxylate

The title compounds were synthesized from 1-tert-butyl 2-methyl 4-oxo-5,6-dihydrocyclopenta[b]pyrrole-1,2(4H)-dicarboxylate (0.53 g, 1.9 mmol) and n-butyllithium in hexanes (10.0 mL, 25.0 mmol, 2.5 M solution) according to General Procedure 4. Purification of the resulting mixture by column chromatography (0-100% EtOAc/heptane), yielded both the BOC-protected product (1-tert-butyl 2-methyl 5-benzyl-4-oxo-5,6-dihydrocyclopenta[b]pyrrole-1,2(4H)-dicarboxylate) and the deprotected methyl 5-benzyl-4-oxo-1,4,5,6-tetrahydrocyclopenta[b]pyrrole-2-carboxylate:

1-tert-Butyl 2-methyl 5-benzyl-4-oxo-5,6-dihydrocyclopenta[b]pyrrole-1,2(4H)-dicarboxylate was isolated as an orange solid (0.098 g, 14% yield). R_{f} (1:1 EtOAc:heptane)=0.46; ¹H NMR (400 MHz, CHLOROFORM-d) δ ppm 1.57 (s, 9H), 2.74-2.77 (m, 1H), 2.84-2.89 (m, 1H), 3.11-3.18 (m, 1H), 3.20-3.26 (m, 1H), 3.34-3.45 (m, 1H), 3.87 (s, 3H), 6.90 (s, 1H), 7.20-7.32 (m, 5H); LCMS-MS (ESI+) 370.1 (M+H).

Methyl 5-benzyl-4-oxo-1,4,5,6-tetrahydrocyclopenta[b]pyrrole-2-carboxylate was isolated as an orange solid (0.150 g, 29% yield). R_{f} (1:1 EtOAc:heptane)=0.30; ¹H NMR (400 MHz, CHLOROFORM-d) δ ppm 2.67-2.77 (m, 2H), 2.94-3.01 (m, 1H), 3.20-3.26 (m, 1H), 3.34-3.40 (m, 1H), 3.88 (s, 3H), 6.99 (s, 1H), 7.20-7.32 (m, 5H).

### 3.3.b) Synthesis of methyl 5-benzyl-1,4,5,6-tetrahydrocyclopenta[b]pyrrole-2-carboxylate

The title compound was synthesized from a mixture of 1-tert-butyl 2-methyl 5-benzyl-4-oxo-5,6-dihydrocyclopenta[b]pyrrole-1,2(4H)-dicarboxylate and methyl 5-benzyl-4-oxo-1,4,5,6-tetrahydrocyclopenta[b]pyrrole-2-carboxylate (0.267 g, 0.99 mmol, using the formula weight of the free pyrrole) according to General Procedure 5. Purification by column chromatography (0-30% EtOAc/heptane) yielded methyl 5-benzyl-1,4,5,6-tetrahydrocyclopenta[b]pyrrole-2-carboxylate as an off-white solid (0.017 g, 7% yield). R_{f} (1:1 EtOAc:heptane)=0.66; ¹H NMR (400 MHz, CHLOROFORM-d) δ ppm 2.30-2.52 (m, 2H), 2.72-2.90 (m, 4H), 3.10-3.25 (m, 1H), 3.82 (s, 3H), 6.64 (s, 1H), 7.21-7.33 (m, 5H).

### 3.4. Synthesis of 5-methyl-1,4,5,6-tetrahydrocyclopenta[b]pyrrole-2-carboxylic acid (21)

The title compound was synthesized from methyl 5,5-difluoro-1,4,5,6-tetrahydrocyclopenta[b]pyrrole-2-carboxylate and lithium hydroxide monohydrate according to General Procedure 7.

### 3.5. Synthesis of 5-methyl-1,4,5,6-tetrahydrocyclopenta[b]pyrrole-2-carboxylic acid (22)

The title compound was synthesized from ethyl 5-methyl-1,4,5,6-tetrahydrocyclopenta[b]pyrrole-2-carboxylate (0.017 g, 0.09 mmol) and lithium hydroxide monohydrate (0.019 g, 0.45 mmol) according to General Procedure 7. The crude was purified by reverse phase HPLC (50-100% MeOH: water, 0.1% formic acid) to give 5-methyl-1,4,5,6-tetrahydrocyclopenta[b]pyrrole-2-carboxylic acid (22) as a light brown solid (2.1 mg, 14%). R_{f} (1:1 EtOAc:heptane)=0.12; ¹H NMR (400 MHz, METHANOL-d₄) δ ppm 1.19 (d, J=6.6, 3H), 2.17-2.20 (m, 1H), 2.26-2.32 (m, 1H), 2.75-2.80 (m, 1H), 2.84-2.90 (m, 2H), 6.56 (s, 1H); LCMS-MS (ESI+) 166.0 (M+H); HPLC (UV=100%), (ELSD=100%).

### 3.6. Synthesis of 5-benzyl-1,4,5,6-tetrahydrocyclopenta[b]pyrrole-2-carboxylic acid (23)

The title compound was synthesized from methyl 5-benzyl-1,4,5,6-tetrahydrocyclopenta[b]pyrrole-2-carboxylate (0.017 g, 0.065 mmol) and lithium hydroxide monohydrate (0.019 g, 0.45 mmol) according to General Procedure 7. The crude product was purified by reverse phase HPLC (40-100% MeOH: water, 0.1% formic acid), to give 5-benzyl-1,4,5,6-tetrahydrocyclopenta[b]pyrrole-2-carboxylic acid (23) as a light brown solid (6.1 mg, 39%). R_{f} (1:1 EtOAc:heptane)=0.12; ¹H NMR (400 MHz, METHANOL-d₄) δ ppm 2.30-2.49 (m, 2H), 2.62-2.90 (m, 4H), 3.08-3.20 (m, 1H), 6.57 (s, 1H), 7.16-7.30 (m, 5H); LCMS-MS (ESI+) 242.3 (M+H); HPLC (UV=100%), (ELSD=100%).

### Example 4

### Synthesis of Pyrrole Analogs with 6-Substituted Fused Cyclopentanes

### 4.1. Synthesis of ethyl 6-oxo-1,4,5,6-tetrahydrocyclopenta[b]pyrrole-2-carboxylate

A solution of the ethyl 1,4,5,6-tetrahydrocyclopenta[b]pyrrole-2-carboxylate (1.0 g, 5.58 mmol) in THF/Water (10:1, 11 mL) at 0° C. was deoxygenated by passing a stream of dry nitrogen gas for 10 min. A solution of 2,3-dichloro-5,6-dicyano-1,4-benzochinone (DDQ) in THF (4 mL) was added dropwise over 5 min. After stirring for 1.5 h, the cooling bath was removed and stirring was continued at rt. Silica gel was added, the solvent stripped off and the silica gel-imbedded material was purified by flash chromatography (0-60% EtOAc/Heptane) to afford ethyl 6-oxo-1,4,5,6-tetrahydrocyclopenta[b]pyrrole-2-carboxylate as a taupe solid (270 mg, 25%). ¹H NMR (400 MHz, CDCl₃) δ ppm 1.39 (t, J=7.13 Hz, 3H), 2.87-2.95 (m, 4H), 4.39 (q, J=7.13 Hz, 2H), 6.78 (d, J=1.66 Hz, 1H), 9.86 (s, 1H); LCMS-MS (ESI+) 193.9 (M+H).

The inventors have determined that the DDQ oxidation in this experiment yielded the 6-oxo instead of the 4-oxo product (see, e.g., oxidation of a cyclopenta[b]pyrrole as described in Quizon-Colquitt, D. M.; Lash, T. D. J. Heterocyclic Chemistry 1993, 30, 477).

### 4.2. Synthesis of methyl 6-oxo-1,4,5,6-tetrahydrocyclopenta[b]pyrrole-2-carboxylate

### 4.2.a) Synthesis of (Z)-methyl 4-(3-tert-butoxy-3-oxoprop-1-enyl)-1H-pyrrole-2-carboxylate and (E)-methyl 4-(3-tert-butoxy-3-oxoprop-1-enyl)-1H-pyrrole-2-carboxylate

Methyl 5-formyl-1H-pyrrole-2-carboxylate (16.3 g) and its regioisomer methyl 4-formyl-1H-pyrrole-2-carboxylate (6.94 g) were obtained (combined yield of 95%) from a Vilsmeier formylation of 1H-pyrrole-2-carboxylic acid methyl ester via exhaustive extraction of the neutralized aqueous layer with EtOAc to provide a better recovery of the more polar 4-formyl isomer [see, e.g., Charkraborty, T. K. et al., Tetrahedron Lett. 2006, 47: 4631 and Denmark, S. E.; Matsuhashi, H. J. Org. Chem. 2002, 67: 3479].

To a suspension of NaH (0.54 g, 13.58 mmol; 60% dispersed in oil) in THF (30 mL) at 0° C. was added (tert-butoxycarbonylmethyl)triphenylphosphonium bromide (6.21 g, 13.585 mmol) as a solid in three portions. The cooling bath was removed and the mixture was stirred at rt for 30 min before cooling to 0° C. Methyl 4-formyl-1H-pyrrole-2-carboxylate (1.6 g, 10.45 mmol) in THF (10 mL) was added dropwise over 10 min. The cooling bath was removed, and the reaction mixture was stirred at rt for about 12 h. The crude product was dried onto silica gel and was purified by flash chromatography (0-20% EtOAc/Heptane) to afford two isomeric compounds:

(Z)-methyl 4-(3-tert-butoxy-3-oxoprop-1-enyl)-1H-pyrrole-2-carboxylate (0.81 g, 26.2%) as a white solid; ¹H NMR (400 MHz, CDCl₃) δ ppm 1.52 (s, 9H), 3.87 (s, 3H), 5.65 (d, J=12.59 Hz, 1H), 6.67 (d, J=12.64 Hz, 1H), 7.25 (dd, J=2.54, 1.56 Hz, 1H), 7.97 (dd, J=3.10, 1.44 Hz, 1H), 9.14 (br s, 1H); LCMS-MS (ESI+) 195.7 (M-56).

(E)-methyl 4-(3-tert-butoxy-3-oxoprop-1-enyl)-1H-pyrrole-2-carboxylate (1.8 g, 57.8%) as a white solid; ¹H NMR (400 MHz, CDCl₃) δ ppm 1.52 (s, 9H), 3.88 (s, 3H), 6.12 (d, J=15.86 Hz, 1H), 7.08 (m, 1H), 7.14 (dd, J=3.03, 1.56 Hz, 1H), 7.49 (d, J=15.86 Hz, 1H), 9.22 (br s, 1H); LCMS-MS (ESI+) 195.8 (M-56).

### 4.2.b) Synthesis of methyl 4-(3-tert-butoxy-3-oxopropyl)-1H-pyrrole-2-carboxylate

To a solution of methyl 4-(3-tert-butoxy-3-oxoprop-1-enyl)-1H-pyrrole-2-carboxylate (2.0 g, 7.96 mmol) in EtOAc (20 mL) under nitrogen was added 10% Pd/C. The flask was evacuated and refilled with hydrogen three times. The reaction mixture was stirred for 2 h. The catalyst was removed by filtration through Celite and the filtrate was concentrated to give methyl 4-(3-tert-butoxy-3-oxopropyl)-1H-pyrrole-2-carboxylate as a white solid (2.02 g, 100%). ¹H NMR (400 MHz, CHLOROFORM-d) δ ppm 1.43 (s, 9H), 2.48 (t, J=7.59 Hz, 2H), 2.77 (t, J=7.54 Hz, 2H), 3.84 (s, 3H), 6.77 (dd, J=2.37, 1.93 Hz, 2H), 930 (br s, 1H); LCMS-MS (ESI+) 198.2 (M-isobutylene).

### 4.2.c) Synthesis of 3-(5-(methoxycarbonyl)-1H-pyrrol-3-yl)propanoic acid

Methyl 4-(3-tert-butoxy-3-oxopropyl)-1H-pyrrole-2-carboxylate (473 g, 1.87 mmol) was treated for about 12 h at rt with 4 N HCl (5 mL). The solvent was removed and the white solid product was dried to give 350 mg (95%) of 3-(5-(methoxycarbonyl)-1H-pyrrol-3-yl)propanoic acid. ¹H NMR (400 MHz, CDCl₃) δ ppm 2.64 (t, J=7.35 Hz, 2H), 2.84 (t, J=7.35 Hz, 2H), 3.85 (s, 3H), 6.79 (dd, J=6.66, 2.22 Hz, 2H), 9.08 (br s, 1H); LCMS-MS (ESI+) 198.2 (M+H).

### 4.2.d) Synthesis of methyl 6-oxo-1,4,5,6-tetrahydrocyclopenta[b]pyrrole-2-carboxylate

To a polyphosphoric acid (115%, 1.6 g) was added 3-(5-(methoxycarbonyl)-1H-pyrrol-3-yl)propanoic acid (174 mg, 0.88 mmol) and 1,2-dichloroethane (8 mL). The reaction mixture was heated for 1 h at 100° C. Water (20 mL) was added and the mixture was carefully poured into a 50 mL Erlenmeyer flask containing solid sodium bicarbonate and ice. The reaction was neutralized (pH 7) and was then extracted with EtOAc (5×50 mL). The combined organic extracts were washed with water, NaHCO₃ and brine, dried (Na₂SO₄) filtered and concentrated. Purification by flash chromatography (0-40% EtOAc/Heptane) afforded methyl 6-oxo-1,4,5,6-tetrahydrocyclopenta[b]pyrrole-2-carboxylate (106 mg, 67%). ¹H NMR (400 MHz, CDCl₃) δ ppm 2.91 (s, 4H), 3.92 (s, 3H), 6.78 (d, J=1.76 Hz, 1H), 9.33 (br s, 1H); LCMS-MS (ESI+) 180.2 (M+H).

### 4.3. Synthesis of ethyl 6-benzyl-1,4,5,6-tetrahydrocyclopenta[b]pyrrole-2-carboxylate

### 4.3.a) Synthesis of ethyl 6-oxo-1-((2-(trimethylsilyl)ethoxy)methyl)-1,4,5,6-tetrahydrocyclopenta[b]pyrrole-2-carboxylate

To sodium hydride (60% dispersion in mineral oil) (0.46 g, 11.4 mmol, 1.1 equiv) in anhydrous DMF (10 mL) under a nitrogen atmosphere at 0° C. was added dropwise a solution ethyl 6-oxo-1,4,5,6-tetrahydrocyclopenta[b]pyrrole-2-carboxylate (2.0 g, 10 mmol, 1 equiv) in anhydrous DMF (15 mL). After stirring for 30 min at 0° C., SEM-Cl (2.1 g, 12.4 mmol, 1.2 equiv) was then added dropwise over 5 min and the mixture warmed to rt overnight. The reaction was quenched by pouring the reaction contents into a beaker of ice water. It was extracted with EtOAc (4×50 mL) and the combined organic extracts were washed with brine, dried (Na₂SO₄), filtered and concentrated. The crude product was purified by flash chromatography (0-30% EtOAc/heptane) to afford 2.9 g of ethyl 6-oxo-1-((2-(trimethylsilyl)ethoxy)methyl)-1,4,5,6-tetrahydrocyclopenta[b]pyrrole-2-carboxylate (86% yield). ¹H NMR (400 MHz, CHLOROFORM-d) δ ppm 6.84 (s, 1H), 5.87 (s, 2H), 4.35 (q, J=7.13 Hz, 2H), 3.57 (dd, J=8.59, 7.71 Hz, 2H), 2.82-2.92 (m, 4H), 1.38 (t, J=7.14 Hz, 3H), 0.85-0.92 (m, 2H), −0.05 (s, 9H).

### 4.3.b) Synthesis of (E/Z)-ethyl 6-benzylidene-1-((2-(trimethylsilyl)ethoxy)methyl)-1,4,5,6-tetrahydrocyclopenta[b]pyrrole-2-carboxylate

The title compound was synthesized from 6-oxo-1-(2-trimethylsilanyl-ethoxymethyl)-1,4,5,6-tetrahydrocyclopenta[b]pyrrole-2-carboxylic acid ethyl ester (0.2 g, 0.62 mmol) and benzylmagnesium chloride (0.7 mL, 2M in THF, 1.36 mmol) according to General Procedure 3. The crude product was semi-purified by flash chromatography (0-20% EtOAc/heptane) to afford 0.14 g of (E/Z)-ethyl 6-benzylidene-1-((2-(trimethylsilyl)ethoxy)methyl)-1,4,5,6-tetrahydrocyclopenta[b]pyrrole-2-carboxylate, which was used in the next step without further purification.

### 4.3.c) Synthesis of (E/Z)-ethyl 6-benzylidene-1,4,5,6-tetrahydrocyclopenta[b]pyrrole-2-carboxylate

To (E/Z)-ethyl 6-benzylidene-1-((2-(trimethylsilyl)ethoxy)methyl)-1,4,5,6-tetrahydrocyclopenta[b]pyrrole-2-carboxylate (0.12 g, 0.3 mmol) was added a solution of tetrabutylammonium fluoride in THF (4 mL, 1M). The vial was capped tightly and heated to 65° C. for 2 h. The reaction mixture was then diluted with a 1:1 mixture of water and brine (30 mL). The resulting aqueous mixture was extracted with EtOAc (4×30 mL). The combined organic phases were washed with brine, dried (Na₂SO₄), filtered and concentrated. The crude product was purified using reverse phase preparative HPLC (methanol/water with 0.1% formic acid and 1% acetonitrile (70%-100%) to afforded 8.1 mg of a 1:1 mixture of (E)- and (Z)-ethyl 6-benzylidene-1,4,5,6-tetrahydrocyclopenta[b]pyrrole-2-carboxylate (10%). LCMS m/e 268 (M+H); ¹H NMR (400 MHz, CDCl₃) δ ppm 8.88 (br. s., 1H), 7.40-7.44 (m, 2H), 7.34-7.39 (m, 2H), 7.18-7.24 (m, 1H), 6.73 (d, J=1.64 Hz, 1H), 6.49 (t, J=2.38 Hz, 1H), 4.35 (q, J=7.11 Hz, 2H), 3.36 (td, J=5.52, 2.55 Hz, 2H), 2.82-2.87 (m, 2H), 1.38 (t, J=7.13 Hz, 3H).

### 4.3.d) Synthesis of ethyl 6-benzyl-1,4,5,6-tetrahydrocyclopenta[b]pyrrole-2-carboxylate

The title compound was synthesized from (E/Z)-ethyl 6-benzylidene-1,4,5,6-tetrahydrocyclopenta[b]pyrrole-2-carboxylate (29.9 mg, 0.11 mmol) according to General Procedure 6 to afford 23.3 mg of ethyl 6-benzyl-1,4,5,6-tetrahydrocyclopenta[b]pyrrole-2-carboxylate in 77% yield. LCMS m/e 292 (M+Na); 268 (M−H); ¹H NMR (400 MHz, CDCl₃) δ ppm 8.30 (br. s., 1H), 7.32-7.38 (m, 2H), 7.25-7.30 (m, 1H), 7.18-7.24 (m, 2H), 6.63 (d, J=1.73 Hz, 1H), 4.26 (q, J=7.11 Hz, 2H), 3.33-3.43 (m, 1H), 2.96 (dd, J=13.39, 6.55 Hz, 1H), 2.76 (dd, J=13.36, 8.99 Hz, 1H), 2.51-2.68 (m, 3H), 2.09-2.21 (m, 1H), 1.32 (t, J=7.13 Hz, 3H).

### 4.4. Synthesis of ethyl 6-phenethyl-1,4,5,6-tetrahydrocyclopenta[b]pyrrole-2-carboxylate

The title compound was synthesized from ethyl 6-oxo-1,4,5,6-tetrahydrocyclopenta[b]pyrrole-2-carboxylate (0.3 g, 1.55 mmol) and phenethylmagnesium bromide (7.5 mL, 0.5 M in THF, 3.7 mmol) according to General Procedure 3 to afford 96.5 mg of (E/Z)-ethyl 6-(2-phenylethylidene)-1,4,5,6-tetrahydrocyclopenta[b]pyrrole-2-carboxylate, followed by General Procedure 6 to give 87.3 mg of ethyl 6-phenethyl-1,4,5,6-tetrahydrocyclopenta[b]pyrrole-2-carboxylate in 90% purity. ¹H NMR (400 MHz, CDCl₃) δ ppm 8.55 (br. s., 1H), 7.29-7.35 (m, 2H), 7.19-7.26 (m, 3H), 6.64 (d, J=1.59 Hz, 1H), 4.23-4.33 (m, 2H), 3.02-3.13 (m, 1H), 2.51-2.82 (m, 5H), 1.94-2.14 (m, 2H), 1.78-1.90 (m, 1H), 1.34 (t, J=7.13 Hz, 3H).

### 4.5. Synthesis of 6-oxo-1,4,5,6-tetrahydro-cyclopenta[b]pyrrole-2-carboxylic acid (24)

The title compound was synthesized from ethyl 6-oxo-1,4,5,6-tetrahydrocyclopenta[b]pyrrole-2-carboxylate (90 mg, 0.47 mmol) and lithium hydroxide monohydrate (78 mg, 1.86 mmol) according to General Procedure 7 (56 mg, 77%). ¹H NMR (400 MHz, CD₃OD) δ ppm 2.84-2.91 (4H), 6.73 (s, 1H); LCMS-MS (ESI+) 165.8 (M+H); HPLC (UV=100%), (ELSD=100%).

### 4.6. Synthesis of (E/Z)-6-benzylidene-1,4,5,6-tetrahydrocyclopenta[b]pyrrole-2-carboxylic acid (25)

The title compound was synthesized from methyl 6-benzylidene-1,4,5,6-tetrahydrocyclopenta[b]pyrrole-2-carboxylate and NaOH according to General Procedure 7. The crude product was purified using preparative HPLC (40%-100% methanol/water with 1% formic acid and 1% acetonitrile) to afford 2.6 mg of a 1:1 mixture of (E)- and (Z)-6-benzylidene-1,4,5,6-tetrahydrocyclopenta[b]pyrrole-2-carboxylic acid (25) in 36% yield. LCMS m/e 240 (M+H). ¹H NMR (400 MHz, methanol-d₄) δ ppm 8.43 (br. s., 1H), 7.42 (d, J=7.64 Hz, 2H), 7.32 (t, J=7.75 Hz, 2H), 7.15 (t, J=7.35 Hz, 1H), 6.68 (t, J=2.25 Hz, 1H), 6.64 (s, 1H), 2.78-2.83 (m, 2H).

### 4.7. Synthesis of 6-benzyl-1,4,5,6-tetrahydrocyclopenta[b]pyrrole-2-carboxylic acid (26)

The title compound was synthesized from ethyl 6-benzyl-1,4,5,6-tetrahydrocyclopenta[b]pyrrole-2-carboxylate and NaOH according to General Procedure 7. The crude product was purified using preparative HPLC (40%-100% methanol/water with 1% formic acid and 1% acetonitrile) to afford 9.9 mg of 6-benzyl-1,4,5,6-tetrahydrocyclopenta[b]pyrrole-2-carboxylic acid (26) in 47% yield. LCMS m/e 264 (M+Na); 240 (M−H). ¹H NMR (400 MHz, methanol-d₄) δ ppm 7.22-7.28 (m, 2H), 7.14-7.20 (m, 3H), 6.54 (s, 1H), 3.33-3.38 (m, 1H), 3.03-3.15 (m, 1H), 2.70 (dd, J=13.45, 8.61 Hz, 1H), 2.35-2.46 (m, 3H), 2.04-2.16 (m, 1H).

### 4.8. Synthesis 6-phenethyl-1,4,5,6-tetrahydrocyclopenta[b]pyrrole-2-carboxylic acid (27)

The title compound was synthesized from ethyl 6-phenethyl-1,4,5,6-tetrahydrocyclopenta[b]pyrrole-2-carboxylate (87.3 mg, 0.31 mmol) and NaOH according to General Procedure 7. The crude product was purified by preparative HPLC (50%-100% methanol/water with 1% formic acid and 1% acetonitrile) to afford 22.6 mg of 6-phenethyl-1,4,5,6-tetrahydrocyclopenta[b]pyrrole-2-carboxylic acid (27) in 29% yield. LCMS m/e 254 (M−H); ¹H NMR (400 MHz, methanol-d₄) δ ppm 7.18-7.28 (m, 4H), 7.11-7.17 (m, 1H), 6.58 (s, 1H), 3.00-3.10 (m, 1H), 2.48-2.71 (m, 5H), 2.00-2.15 (m, 2H), 1.66-1.79 (m, 1H).

The enantiomers of 6-phenethyl-1,4,5,6-tetrahydrocyclopenta[b]pyrrole-2-carboxylic acid were separated according to General Procedure 8 using 25% of a mixture of 50:50 methanol/isopropanol in CO₂ with 0.2% diethylamine to give 6-phenethyl-1,4,5,6-tetrahydrocyclopenta[b]pyrrole-2-carboxylic acid (28) (peak 2, retention time=10.2 min; 97% ee) and 6-phenethyl-1,4,5,6-tetrahydrocyclopenta[b]pyrrole-2-carboxylic acid (29) (peak 1, retention time=9.2 min; 99% ee).

### Example 5

### Synthesis of Pyrrole Analogs with Fused Cyclohexanes

### 5.1. Synthesis of methyl 4-benzyl-4,5,6,7-tetrahydro-1H-indole-2-carboxylate

### 5.1.a) Synthesis of ethyl 4-oxo-4,5,6,7-tetrahydro-1H-indole-2-carboxylate

Methanol hydrochloride (60 mL) was added to a solution of 4-oxo-4,5,6,7-tetrahydro-1H-indole-2-carbonitrile (Estep, K. G. Syn. Commun. 1995, 25, 507-514) (0.665 g, 4.15 mmol) in MeOH (10 mL) and the resulting solution was refluxed overnight. The solvent was removed in vacuo and saturated NaHCO₃ was added. An approximately equal volume of EtOAc was added. The organic layer was then removed and dried over sodium sulfate, filtered and evaporated to give 0.675 g of a solid consisting of methyl 4-oxo-4,5,6,7-tetrahydro-1H-indole-2-carboxylate (80%) and starting material (20%). ¹H-NMR (400 MHz, CDCl₃) δ ppm 2.19 (m, 2H), 2.53 (m, 2H), 2.88 (m, 2H), 3.88 (s, 3H), 7.21 (d, 1H), 9.53 (s broad, 1H); ¹³C-NMR (100 MHz, CDCl₃) δ ppm 22.85, 23.44, 37.96, 51.92, 108.21, 112.38, 161.74, 194.33; DEPT (100 MHz, CDCl₃) δ ppm CH₃ carbons: 51.92; CH₂ carbons: 22.85, 23.44, 37.96; CH carbons: 112.38; LC/MS: 94.09%, m/z=193.

### 5.1b) Synthesis of methyl-4-oxo-1-((2-(trimethylsilyl)ethoxy)methyl)-4,5,6,7-tetrahydro-1H-indole-2-carboxylate

A solution of methyl 4-oxo-4,5,6,7-tetrahydro-1H-indole-2-carboxylate (500 mg, 2.6 mmol) in DMF (3 mL) was added to a cooled (0° C.) suspension of sodium hydride (114 mg, 60% in oil, 2.8 mmol) in DMF (2 mL). After 10 min, 2-(trimethylsilyl)ethoxymethyl chloride (SEM-Cl) (550 μl, 3.1 mmol) was added. The mixture was stirred at rt for 2 h and was then poured into ice-water and extracted with EtOAc. After concentration, methyl 4-oxo-1-((2-(trimethylsilyl)ethoxy)methyl)-4,5,6,7-tetrahydro-1H-indole-2-carboxylate was obtained as a crude oil (930 mg). ¹H NMR (CDCl₃, 400 MHz) δ ppm 7.27 (1H, s), 5.78 (2H, s), 3.61 (2H, m), 2.94 (2H, m), 2.50 (2H, m), 2.18 (2H, m), 0.92 (2H, m); LC/MS: 60%.

### 5.1.c) Synthesis of (E)-methyl 4-benzylidene-1-((2-(trimethylsilyl)ethoxy)methyl)-4,5,6,7-tetrahydro-1H-indole-2-carboxylate

The title compound was synthesized from methyl 4-oxo-1-((2-(trimethylsilyl)ethoxy)methyl)-4,5,6,7-tetrahydro-1H-indole-2-carboxylate (900 mg, 2.78 mmol) and benzylmagnesium chloride (3.4 mL, 2M in THF, 6.8 mmol) according to General Procedure 3. In this example, additional benzylmagnesium chloride (1.7 mL, 2M in THF, 3.4 mmol) was added after 2 h. The crude product (900 mg) was used in the next step without further purification. LC/MS: 50%, m/z=397 g/mol.

### 5.1.d) Synthesis of (E)-methyl 4-benzylidene-4,5,6,7-tetrahydro-1H-indole-2-carboxylate

Tetrabutylammonium fluoride (TBAF) (23 mL, 1M in THF, 23 mmol) was added over 5 min to a solution of (E)-methyl 4-benzylidene-1-((2-(trimethylsilyl)ethoxy)methyl)-4,5,6,7-tetrahydro-1H-indole-2-carboxylate (900 mg, 2.26 mmol) in cooled THF (0° C.). The reaction mixture was then heated for 4 h at 80° C. After 48 h at rt, the reaction mixture was partitioned between ether and water. The organic layer was dried over MgSO₄ and concentrated under reduced pressure. The crude product was purified by silica gel chromatography (cyclohexane/EtOAc: 80/20) to afford the (E)-methyl 4-benzylidene-4,5,6,7-tetrahydro-1H-indole-2-carboxylate (80 mg). LC/MS: 76%, m/z=267 g/mol.

### 5.1.e) Synthesis of ethyl 4-benzyl-4,5,6,7-tetrahydro-1H-indole-2-carboxylate

The title compound was synthesized from (E)-methyl 4-benzylidene-4,5,6,7-tetrahydro-1H-indole-2-carboxylate according to General Procedure 6. The crude product was purified by silica gel chromatography (cyclohexane/CH₂Cl₂: 50/50). LC/MS: 60%, m/z=269 g/mol.

### 5.2. Synthesis of 4-benzyl-4,5,6,7-tetrahydro-1H-indole-2-carboxylic acid (32)

The title compound was synthesized from methyl 4-benzyl-4,5,6,7-tetrahydro-1H-indole-2-carboxylate (20 mg, 0.08 mmol) and aqueous NaOH (1M in H₂O, 0.8 mL, 0.8 mmol) according to General Procedure 7. The solid was filtered off, washed with water and dried under vacuum for about 12 h to give 4-benzyl-4,5,6,7-tetrahydro-1H-indole-2-carboxylic acid (32) (19 mg). ¹H NMR (CDCl₃, 400 MHz) δ ppm 8.8 (1H, br s), 7.3-7.33 (2H, m), 7.2-7.26 (3H, m), 6.81 (1H, s), 3.09 (1H, dd), 2.9 (1H, m), 2.55-2.65 (3H, m), 1.9-2.0 (1H, m), 1.6-1.8 (2H, m), 1.3-1.4 (1H, m); LC/MS: 89%, m/z=255 g/mol.

### Example 6

### D-Amino Acid Oxidase Inhibition

### 6.1. D-Amino Acid Oxidase Enzyme Assay

DAAO enzyme activity was measured using the substrate D-serine at its Michaelis-Menton Kₘ of 5 mM. The rate of oxidation is measured as a rate of production of hydrogen peroxide, which was detected using the enzyme horseradish peroxidase (Sigma cat. No. P-8375). This coupled reaction uses the enzyme substrate Amplex Red (Molecular Probes), which is converted to the fluorescent reaction product, resorufin (excitation 530-560 nm; emission 590 nm). Although DAAO has a higher pH optimum, all reagents were prepared in 50 mM sodium phosphate buffer at pH 7.4 and inhibition curves were generated at this pH.

The final concentrations of components in 200 μl total volume per well (black clear-bottom 96-well plate, Costar) were:

(a) Horseradish peroxidase: 4 Units per mL

### (b) D-serine: 5 mM

### (c) Test Compound: 100-0.0064 uM for IC₅₀s

(d) Amplex Red reagent: 50 uM

### (e) DMSO: 1.6%

The reactions were initiated by addition of DAAO enzyme and the fluorescence was monitored. H₂O₂ was added at 16 uM final concentration to a control well on each plate to test for compound interference with a coupled enzyme. Inhibition curves were generated in the presence of varying concentrations of the inhibitor and IC₅₀ values were calculated for each inhibitor.

### 6.2. Results of DAAO Inhibition Assay

IC₅₀ values were determined for compounds I through 37, which are summarized in Table 2 below.

TABLE 2
Human and Porcine DAAO Inhibition [IC₅₀]

| Compound | | Human DAAO |
|---|---|---|
| No. | Compound Name | (μM) |
| 1 | 1,4,5,6-tetrahydrocyclopenta[b]pyrrole-2-carboxylic acid | (+++) |
| 2 | 4-oxo-1,4,5,6-tetrahydrocyclopenta[b]pyrrole-2-carboxylic acid | (+) |
| 3 | 3-tert-butyl-4-oxo-1,4,5,6-tetrahydrocyclopenta[b]pyrrole-2- | 28% @ 100 |
| | carboxylic acid | |
| 4 | 4-methyl-1,4,5,6-tetrahydrocyclopenta[b]pyrrole-2-carboxylic | (++) |
| | acid | |
| 5^{a)} | 4-methyl-1,4,5,6-tetrahydrocyclopenta[b]pyrrole-2-carboxylic | (++) |
| | acid (enantiomer 1) | |
| 6^{b)} | 4-methyl-1,4,5,6-tetrahydrocyclopenta[b]pyrrole-2-carboxylic | (+) |
| | acid (enantiomer 2) | |
| 7 | 4-propyl-1,4,5,6-tetrahydrocyclopenta[b]pyrrole-2-carboxylic | (+++) |
| | acid | |
| 8^{a)} | 4-propyl-1,4,5,6-tetrahydrocyclopenta[b]pyrrole-2-carboxylic | (+++) |
| | acid (enantiomer 1) | |
| 9^{b)} | 4-propyl-1,4,5,6-tetrahydrocyclopenta[b]pyrrole-2-carboxylic | (+) |
| | acid (enantiomer 2) | |
| 10 | 4-isopropyl-1,4,5,6-tetrahydrocyclopenta[b]pyrrole-2- | (++) |
| | carboxylic acid | |
| 11 | 4-isobutyl-1,4,5,6-tetrahydrocyclopenta[b]pyrrole-2-carboxylic | (++) |
| | acid | |
| 12 | 4-(cyclohexylmethyl)-1,4,5,6-tetrahydrocyclopenta[b]pyrrole-2- | (++) |
| | carboxylic acid | |
| 13 | 4-(4-fluorobenzylidene)-1,4,5,6-tetrahydrocyclopenta[b]pyrrole- | (+) |
| | 2-carboxylic acid | |
| 14 | 4-(4-chlorobenzyl)-1,4,5,6-tetrahydrocyclopenta[b]pyrrole-2- | (+++) |
| | carboxylic acid | |
| 15^{a)} | 4-(4-chlorobenzyl)-1,4,5,6-tetrahydrocyclopenta[b]pyrrole-2- | (+++) |
| | carboxylic acid (enantiomer 1) | |
| 16^{b)} | 4-(4-chlorobenzyl)-1,4,5,6-tetrahydrocyclopenta[b]pyrrole-2- | (+) |
| | carboxylic acid (enantiomer 2) | |
| 17 | 4-benzyl-1,4,5,6-tetrahydrocyclopenta[b]pyrrole-2-carboxylic | (+++) |
| | acid | |
| 33 | 4-(4-isopropylbenzyl)-1,4,5,6-tetrahydrocyclopenta[b]pyrrole-2- | (++) |
| | carboxylic acid | |
| 18 | 4-phenethyl-1,4,5,6-tetrahydrocyclopenta[b]pyrrole-2- | (++) |
| | carboxylic acid | |
| 19^{a)} | 4-phenethyl-1,4,5,6-tetrahydrocyclopenta[b]pyrrole-2- | (++) |
| | carboxylic acid (enantiomer 1) | |
| 20^{b)} | 4-phenethyl-1,4,5,6-tetrahydrocyclopenta[b]pyrrole-2- | (+) |
| | carboxylic acid (enantiomer 2) | |
| 34 | 4-(4-methoxyphenethyl)-1,4,5,6- | (+) |
| | tetrahydrocyclopenta[b]pyrrole-2-carboxylic acid | |
| 35 | 4-(2-methyl-2-phenylpropyl)-1,4,5,6- | (+) |
| | tetrahydrocyclopenta[b]pyrrole-2-carboxylic acid | |
| 36 | 4-(3-phenylpropyl)-1,4,5,6-tetrahydrocyclopenta[b]pyrrole-2- | (++) |
| | carboxylic acid | |
| 37 | 4-p-tolyl-1,4,5,6-tetrahydrocyclopenta[b]pyrrole-2-carboxylic | (++) |
| | acid | |
| 21 | 5,5-difluoro-1,4,5,6-tetrahydrocyclopenta[b]pyrrole-2- | (++) |
| | carboxylic acid | |
| 22 | 5-methyl-1,4,5,6-tetrahydrocyclopenta[b]pyrrole-2-carboxylic | (++) |
| | acid | |
| 23 | 5-benzyl-1,4,5,6-tetrahydrocyclopenta[b]pyrrole-2-carboxylic | (+) |
| | acid | |
| 24 | 6-oxo-1,4,5,6-tetrahydrocyclopenta[b]pyrrole-2-carboxylic acid | (++) |
| 26 | 6-benzyl-1,4,5,6-tetrahydrocyclopenta[b]pyrrole-2-carboxylic | (+++) |
| | acid | |
| 27 | 6-phenethyl-1,4,5,6-tetrahydrocyclopenta[b]pyrrole-2- | (++) |
| | carboxylic acid | |
| 28^{c)} | 6-phenethyl-1,4,5,6-tetrahydrocyclopenta[b]pyrrole-2- | (++) |
| | carboxylic acid (enantiomer 1) | |
| 29^{d)} | 6-phenethyl-1,4,5,6-tetrahydrocyclopenta[b]pyrrole-2- | (+) |
| | carboxylic acid (enantiomer 2) | |
| 30 | 4,5,6,7-tetrahydro-1H-indole-2-carboxylic acid | (++) |
| 31 | 3-methyl-4-oxo-4,5,6,7-tetrahydro-1H-indole-2-carboxylic acid | (+) |
| 32 | 4-benzyl-4,5,6,7-tetrahydro-1H-indole-2-carboxylic acid | (+)* |

IC₅₀ ≦ 100 nM = (+++); IC₅₀ ≦ 1 μM = (++); IC₅₀ ≦ 100 μM = (+)
*porcine data
For those compounds of Table 2 marked with ^{a)}, ^{b)}, ^{c)} or ^{d)} absolute stereochemistries are assumed based on docking studies in the D-amino acid oxidase active site (see e.g., Protein Science 2006, 15(12), 2708-2717 and Biochemical and Biophysical Research Communications 2007, 355(2), 385-391, and references cited within) for crystal structures:

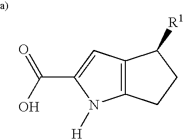

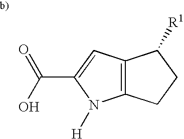

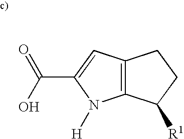

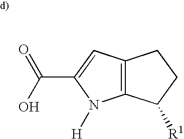

These data demonstrate that the above described method can be used to identify compounds that are DAAO inhibitors. The method can also be used to determine the efficacy of such compounds, e.g., the IC₅₀ of such compounds (for example, IC₅₀ less than or equal to 100 nM; less than or equal to 1 uM, or less than or equal to 100 uM).

### Example 7

### In Vivo Elevation of D-Serine Levels in the Cerebellum

### 7.1. Methods

Mice (C57BL/6, 8-9 weeks of age) are dosed intraperitoneally at 10 mL/kg with 50 mg/kg of compound suspended in 45% (w/v) hydroxy-β-cyclodextrin vehicle. Animals are sacrificed at either 2 or 6 h post compound administration with an N=3 per time point. At sacrifice, trunk blood is collected into tubes containing potassium EDTA, which are then centrifuged to permit isolation of plasma. The cerebellum is dissected from each animal. Plasma and cerebellum samples are stored at −80° C. until samples are analyzed (LC/MS/MS).

### 7.2. Results

Results obtained for compound I are summarized in Table 3, below.

TABLE 3
In vivo elevation of D-serine levels in the cerebellum

| | | | Avg D-serine |
|---|---|---|---|
| | | | Level in |
| Compound | | Time | Cerebellum |
| No. | Compound Name | (h) | (nmol/g) |
| | Vehicle | 2 | 2.1 |
| 1 | 1,4,5,6-tetrahydrocyclo- | 2 | ++ |
| | penta[b]pyrrole- | 6 | + |
| | 2-carboxylic acid | | |

>5 = (++); 2.5-4.9 = (+); <2.5 = (−)

These data demonstrate that compounds of the invention can be used to increase the concentration of D-serine in the brain (e.g., cerebellum) of a mammal. In addition, the method can be used to identify compounds that are DAAO inhibitors effective for increasing D-serine in the brain (e.g., cerebellum).

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

What is claimed is:

1. A compound having a structure according to Formula (VI): 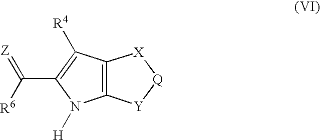
wherein
Z is a member selected from O and S;
X, Q and Y are members independently selected from —CR¹R²—, C═O, C═S, C═NR³ and C═CR⁴⁰R⁴¹,
with the proviso that at least one of X, Q and Y is other than —CH₂—,
wherein
X and Q are optionally joined to form a 3-, 4- or 5-membered ring;
Y and Q are optionally joined to form a 3-, 4- or 5-membered ring;
X and Y, together with the atoms to which they are attached, are optionally joined to form a 5- to 7-membered ring thereby forming a bicyclic substructure;
R³ is a member selected from H, OR¹², acyl, NR¹²R¹³, SO₂R¹³, SOR¹³ substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R¹² and R¹³ are members independently selected from substituted or unsubstituted alkyl, substituted or unsubstituted heteroalkyl, substituted or unsubstituted aryl, substituted or unsubstituted heteroaryl and substituted or unsubstituted heterocycloalkyl;
R⁴ is a member selected from H, CF₃, F, Cl, Br, CN, OR¹⁴, NR¹⁴R¹⁵, C₄-C₆ unsubstituted alkyl, substituted or unsubstituted cycloalkyl, substituted or unsubstituted heterocycloalkyl, substituted or unsubstituted arylalkyl, substituted or unsubstituted heteroarylalkyl, cycloalkyl-substituted alkyl and heterocycloalkyl-substituted alkyl,
wherein
R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl;
each R¹, each R², each R⁴⁰ and each R⁴¹ is a member independently selected from H, halogen, CN, CF₃, acyl, C(O)OR^{14′}, C(O)NR^{14′}R^{15′}, OR^{14′}, S(O)₂OR^{14′}, S(O)ₚR^{14′}, SO₂NR^{14′}R^{15′}, NR^{14′}R^{15′}, NR^{14′}C(O)R^{15′}, NR^{14′}S(O)₂R^{15′}, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
adjacent R¹ and R², together with the atoms to which they are attached, are optionally joined to form a 3-, 4- or 5-membered ring;
p is an integer selected from 0 to 2;
R^{14′} and R^{15′} are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, wherein R¹⁴ and R¹⁵, together with the atoms to which they are attached, are optionally joined to form a 5- to 7-membered ring; and
R⁶ is a member selected from OH and O⁻X⁺, wherein X⁺ is a cation,
and any enantiomer, diastereoisomer, racemic mixture, enantiomerically enriched mixture, and enantiomerically pure form thereof.

2. The compound according to claim 1, wherein at least one of R¹, R², R³, R⁴⁰ and R⁴¹ has the formula: 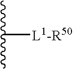
wherein
R⁵⁰ is a member selected from substituted or unsubstituted aryl, substituted or unsubstituted heteroaryl, substituted or unsubstituted cycloalkyl, substituted or unsubstituted heterocycloalkyl and a fused ring system; and
L¹ is a linker moiety, which is a member selected from substituted or unsubstituted alkyl, substituted or unsubstituted heteroalkyl, substituted or unsubstituted aryl, substituted or unsubstituted heteroaryl and substituted or unsubstituted heterocycloalkyl.

3. The compound according to claim 2, wherein at least one of R¹, R² and R³ has a formula, which is a member selected from: 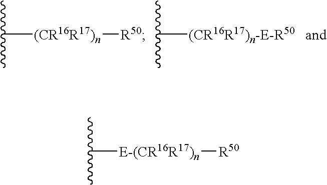
wherein
n is an integer from 1 to 5;
each E is a member independently selected from —O—, —S—, —NR⁴³—, —C(O)NR⁴³—, —NR⁴³C(O)—, —S(O)₂NR⁴³— and —NR⁴³S(O)₂—, wherein each R⁴³ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and
R¹⁶ and R¹⁷ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
two of R¹, R¹⁶ and R¹⁷ or two of R², R¹⁶ and R¹⁷, together with the carbon atoms to which they are attached, are optionally joined to form a 3- to 7-membered ring, wherein said ring is a member selected from substituted or unsubstituted cycloalkyl and substituted or unsubstituted heterocycloalkyl, and wherein said ring is optionally fused to R⁵⁰.

4. The compound according to claim 3, wherein (CR¹⁶R¹⁷)ₙ is a member selected from —CH₂—, —CH₂CH₂— and —CH₂CH₂CH₂—.

5. The compound according to claim 3, wherein R⁵⁰ is a member selected from substituted or unsubstituted aryl and substituted or unsubstituted heteroaryl.

6. The compound according to claim 5, wherein said substituted or unsubstituted aryl has the formula: 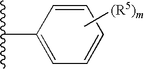
wherein
m is an integer from 0 to 5; and
each R⁵ is a member independently selected from H, halogen, CN, CF₃ hydroxy, alkoxy, acyl, C(O)OR¹⁸, OC(O)R¹⁸, NR¹⁸R¹⁹, C(O)NR¹⁸R¹⁹, NR¹⁸C(O)R²⁰, NR¹⁸SO₂R²⁰, S(O)₂R²⁰, S(O)R²⁰, substituted or unsubstituted alkyl, substituted or unsubstituted heteroalkyl, substituted or unsubstituted aryl, substituted or unsubstituted heteroaryl and substituted or unsubstituted heterocycloalkyl, wherein adjacent R⁵, together with the atoms to which they are attached, are optionally joined to form a ring, wherein said ring is a member selected from substituted or unsubstituted cycloalkyl, substituted or unsubstituted heterocycloalkyl, substituted or unsubstituted aryl and substituted or unsubstituted heteroaryl,
wherein
R¹⁸ and R¹⁹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl;
R²⁰ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl;
and two of R¹⁸, R¹⁹ and R²⁰, together with the atoms to which they are attached, are optionally joined to form a 5- to 7-membered ring.

7. The compound according to claim 1, having a formula, which is a member selected from:

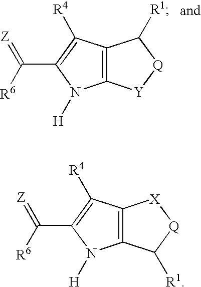

8. The compound of claim 7 having a formula, which is a member selected from:

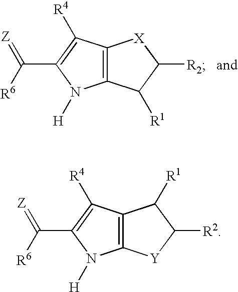

9. The compound of claim 8 having a structure selected from: 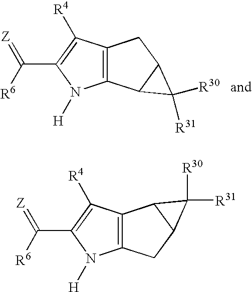
wherein R³⁰ and R³¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

10. The compound according to claim 9, wherein at least one of R³⁰ and R³¹ has the formula: 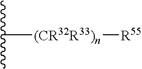
wherein
R⁵⁵ is a member selected from substituted or unsubstituted aryl, substituted or unsubstituted heteroaryl, substituted or unsubstituted cycloalkyl and substituted or unsubstituted heterocycloalkyl;
each n is an integer from 0 to 5; and
each R³² and each R³³ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R³² and R³³, together with the carbon atom to which they are attached, are optionally joined to form a 3- to 7-membered ring, which is optionally fused to R⁵⁵.

11. The compound of claim 1 having the formula: 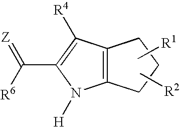
wherein
at least one of R¹ and R² is other than H; and
adjacent R¹ and R², together with the atoms to which they are attached, are optionally joined to form a 3-, 4- or 5-membered ring.

12. The compound of claim 11 having a formula, which is a member selected from: 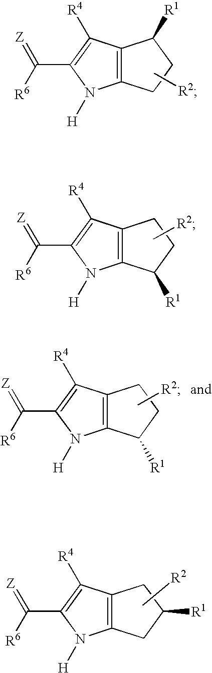
wherein R¹ is other than H.

13. The compound of claim 12, wherein R¹ is substituted or unsubstituted alkyl.

14. The compound of claim 13, wherein R¹ is a member selected from substituted or unsubstituted methyl, substituted or unsubstituted ethyl, substituted or unsubstituted n-propyl, substituted or unsubstituted iso-propyl, substituted or unsubstituted n-butyl and substituted or unsubstituted iso-butyl.

15. The compound of claim 13, wherein R¹ is aryl-substituted alkyl or heteroaryl-substituted alkyl.

16. The compound of claim 13, wherein said alkyl is substituted with a member selected from substituted or unsubstituted cycloalkyl and substituted or unsubstituted heterocycloalkyl.

17. The compound according to claim 1, wherein at least one of X, Q and Y is CHF or CF₂.

18. The compound according to claim 17, having a formula, which is a member selected from:

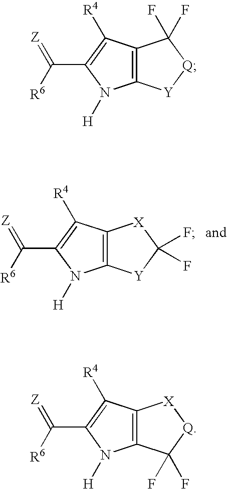

19. The compound according to claim 1, wherein Z is O.

20. The compound according to claim 1, wherein R¹ and R² are members independently selected from H, F, methyl, ethyl, n-propyl, iso-propyl, n-butyl, iso-butyl, substituted or unsubstituted arylalkyl, substituted or unsubstituted heteroarylalkyl, cycloalkyl-substituted alkyl and heterocycloalkyl-substituted alkyl.

21. A pharmaceutical composition comprising a compound of claim 1, or a pharmaceutically acceptable salt thereof, and a pharmaceutically acceptable carrier.

22. A composition comprising a first stereoisomer and at least one additional stereoisomer of a compound according to claim 1, wherein said first stereoisomer is present in an enantiomeric or diastereomeric excess of at least 80% relative to said at least one additional stereoisomer.

23. A method for treating or preventing a condition which is a member selected from a neurological disorder, pain, ataxia and convulsion, said method comprising administering to a subject in need thereof a therapeutically effective amount of a compound according to Formula (I): 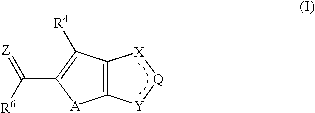
wherein
Z is a member selected from O and S;
A is a member selected from NR⁷, S and O;
X, Q and Y are members independently selected from O, S, NR³, CR¹, —(CR¹R²)_{q}—, C═O, C═S, C═NR³ and C═CR⁴⁰R⁴¹, wherein q is an integer selected from 1 and 2,
with the proviso that the ring, which includes Q, X and Y is a non-aromatic ring,
wherein
X and Q are optionally joined to form a 3- to 7-membered ring;
Y and Q are optionally joined to form a 3- to 7-membered ring;
X and Y, together with the atoms to which they are attached, are optionally joined to form a 5- to 7-membered ring thereby forming a bicyclic substructure; and
R³ and R⁷ are members independently selected from H, OR¹², acyl, NR¹²R¹³, SO₂R¹³, SOR¹³, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R¹² and R¹³ are members independently selected from substituted or unsubstituted alkyl, substituted or unsubstituted heteroalkyl, substituted or unsubstituted aryl, substituted or unsubstituted heteroaryl and substituted or unsubstituted heterocycloalkyl;
R⁴, each R¹, each R², each R⁴⁰ and each R⁴¹ are members independently selected from H, halogen, CN, CF₃, acyl, C(O)OR¹⁴, C(O)NR¹⁴R¹⁵, OR¹⁴, S(O)₂OR¹⁴, S(O)ₚR¹⁴SO₂NR¹⁴R¹⁵, NR¹⁴R¹⁵, NR¹⁴C(O)R¹⁵, NR¹⁴S(O)₂R¹⁵, substituted or unsubstituted alkyl, substituted or unsubstituted heteroalkyl, substituted or unsubstituted aryl, substituted or unsubstituted heteroaryl and substituted or unsubstituted heterocycloalkyl, and R¹ and R², together with the atoms to which they are attached, are optionally joined to form a 3- to 7-membered ring,
wherein
p is an integer selected from 0 to 2;
R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and
R¹⁴ and R¹⁵, together with the atoms to which they are attached, are optionally joined to form a 5- to 7-membered ring; and
R⁶ is a member selected from OR⁸, O⁻X⁺, NR⁹R¹⁰, NR⁹NR^{9′}R¹⁰,NR⁹OR¹⁰, NR⁹SO₂R¹¹, wherein X⁺ is a cation; and R⁶ and R⁷, together with the atoms to which they are attached, are optionally joined to form a 5- to 7-membered ring,
wherein
R⁸ is a member selected from the group consisting of H, substituted or unsubstituted alkyl, substituted or unsubstituted heteroalkyl, substituted or unsubstituted aryl, substituted or unsubstituted heteroaryl, substituted or unsubstituted heterocycloalkyl and a single negative charge;
R⁹, R^{9′} and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl;
R¹¹ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl; and
at least two of R⁸, R⁹, R^{9′}, R¹⁰ and R¹¹, together with the atoms to which they are attached, are optionally joined to form a 5- to 7-membered ring,
and any enantiomer, diastereoisomer, racemic mixture, enantiomerically enriched mixture, and enantiomerically pure form thereof.

24. The method according to claim 23, wherein A is NR⁷.

25. The method according to claim 24, wherein R⁷ is H.

26. The method according to claim 23, wherein R⁶ is OR⁸ or O⁻X⁺.

27. The method according to claim 26, wherein R⁸ is a member selected from H and a single negative charge.

28. The method according to claim 23, wherein R¹ and R² are members independently selected from H, F, methyl, ethyl, n-propyl, iso-propyl, n-butyl, iso-butyl, substituted or unsubstituted arylalkyl, substituted or unsubstituted heteroarylalkyl, substituted or unsubstituted cycloalkyl-substituted alkyl and substituted or unsubstituted heterocycloalkyl-substituted alkyl.

29. The method according to claim 23, wherein at least one of X, Q and Y is other than —CH₂—.

30. The method according to claim 23, wherein said compound has a structure according to Formula (VI): 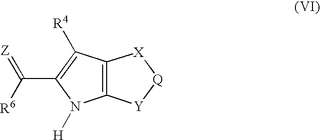
wherein
Z is a member selected from O and S;
X, Q and Y are members independently selected from —CR¹R²—, C═O, C═S, C═NR³ and C═CR⁴⁰R⁴¹,
with the proviso that at least one of X, Q and Y is other than —CH₂—,
wherein
X and Q are optionally joined to form a 3-, 4- or 5-membered ring;
Y and Q are optionally joined to form a 3-, 4- or 5-membered ring;
X and Y, together with the atoms to which they are attached, are optionally joined to form a 5- to 7-membered ring thereby forming a bicyclic substructure;
R³ is a member selected from H, OR¹², acyl, NR¹²R¹³, SO₂R¹³, SOR¹³, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R¹² and R¹³ are members independently selected from substituted or unsubstituted alkyl, substituted or unsubstituted heteroalkyl, substituted or unsubstituted aryl, substituted or unsubstituted heteroaryl and substituted or unsubstituted heterocycloalkyl;
R⁴ is a member selected from H, CF₃, F, Cl, Br, CN, OR¹⁴, NR¹⁴R¹⁵, C₄-C₆ unsubstituted alkyl, substituted or unsubstituted cycloalkyl, substituted or unsubstituted heterocycloalkyl, substituted or unsubstituted arylalkyl, substituted or unsubstituted heteroarylalkyl, cycloalkyl-substituted alkyl and heterocycloalkyl-substituted alkyl,
wherein
R¹⁴ and R¹⁵ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl;
each R¹, each R², each R⁴⁰ and each R⁴¹ is a member independently selected from H, halogen, CN, CF₃, acyl, C(O)OR^{14′}, C(O)NR^{14′}R^{15′}, OR^{14′}, S(O)₂OR^{14′}, S(O)ₚR^{14′}, SO₂NR^{14′}R^{15′}, NR^{14′}R^{15′}, NR^{14′}C(O)R^{15′}, NR^{14′}S(O)₂R^{15′}, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R¹ and R², together with the atoms to which they are attached, are optionally joined to form a 3-, 4- or 5-membered ring;
p is an integer selected from 0 to 2;
R^{14′} and R^{15′} are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, wherein R¹⁴ and R¹⁵, together with the atoms to which they are attached, are optionally joined to form a 5- to 7-membered ring; and
R⁶ is a member selected from OH and O⁻X⁺, wherein X⁺ is a cation,
and any enantiomer, diastereoisomer, racemic mixture, enantiomerically enriched mixture, and enantiomerically pure form thereof.

31. The method according to claim 23, wherein said neurological disorder is a neurodegenerative disease.

32. The method according to claim 31, wherein said neurodegenerative disease is a member selected from Alzheimer's disease, Parkinson's disease and amyotrophic lateral sclerosis.

33. The method according to claim 23, wherein said neurological disorder is a neuropsychiatric disease.

34. The method according to claim 33, wherein said neuropsychiatric disease is schizophrenia.

35. The method according to claim 23, wherein said pain is neuropathic pain.

36. The method according to claim 23, wherein said pain is a member selected from diabetic neuropathy, post-herpetic neuralgia, spinal cord injury induced pain, neuropathic cancer pain, HIV/AIDS induced pain, phantom limb pain, trigeminal neuralgia, complex regional pain syndrome, chronic migraine, fibromyalgia and lower back pain.

37. The method according to claim 23, further comprising co-administering to said subject at least one member selected from gabapentin and pregabalin.

38. The method according to claim 23, further comprising co-administering to said subject a modulator of NMDA neurotransmission.

39. The method according to claim 38, wherein said modulator is a member selected from D-serine, cycloserine and analogs thereof.

40. A method of enhancing cognition in a mammalian subject, said method comprising administering to said subject an effective amount of a compound according to claim 1, or a pharmaceutically acceptable salt, solvate or prodrug thereof.

41. The method according to claim 40, wherein said subject has been diagnosed with a neurological disorder.

42. The method according to claim 41, wherein said neurological disorder is a neurodegenerative disease.

43. The method according to claim 40, wherein said subject has been diagnosed with brain injury or spinal cord injury.

44. The method according to claim 40, wherein said subject is in need of relieving negative symptoms of stress, sleep deprivation or disruption of the circadian rhythm.

45. A method of inhibiting D-amino acid oxidase (DAAO) activity, said method comprising contacting said DAAO with a compound of claim 1.

46. The method of claim 45, wherein said DAAO is located within a mammalian cell.

47. The method of claim 46, wherein said mammalian cell is located in central or peripheral nervous system of a mammal.

48. A method of increasing D-serine level in brain of a mammal, said method comprising administering to said mammal an effective amount of a compound of claim 1.
